SEQUENCE LISTING (23 sequences)

SEQ ID NO: 1 - protein, 10 aa, Artificial Sequence
  Heavy chain CDR1
GFTFSHYTLS

SEQ ID NO: 2 - protein, 17 aa, Artificial Sequence
  Heavy chain CDR2
VISGDGSYTYYADSVKG

SEQ ID NO: 3 - protein, 9 aa, Artificial Sequence
  18R5 Heavy chain CDR3
NFIKYVFAN

SEQ ID NO: 4 - protein, 11 aa, Artificial Sequence
  Light chain CDR1
SGDNIGSFYVH

SEQ ID NO: 5 - protein, 8 aa, Artificial Sequence
  Light chain CDR2
DKSNRPSG

SEQ ID NO: 6 - protein, 9 aa, Artificial Sequence
  Light chain CDR3
QSYANTLSL

SEQ ID NO: 7 - protein, 118 aa, Artificial Sequence
  Heavy chain variable region amino acid sequence
EVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSS

SEQ ID NO: 8 - protein, 108 aa, Artificial Sequence
  Light chain variable region amino acid sequence
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLG

SEQ ID NO: 9 - protein, 463 aa, Artificial Sequence
  Heavy chain amino acid sequence
  SIGNAL (1)..(19)
MKHLWFFLLLVAAPRWVLSEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 232 aa, Artificial Sequence
  Light chain amino acid sequence
  SIGNAL (1)..(19)
MAWALLLLTLLTQGTGSWADIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 444 aa, Artificial Sequence
  Heavy chain amino acid sequence
EVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 213 aa, Artificial Sequence
  Light chain amino acid sequence
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 8 aa, Artificial Sequence
  FLAG peptide
DYKDDDDK

SEQ ID NO: 14 - DNA, 1200 bp, Artificial Sequence
  Human LEF1
atgccccaactctccggaggaggtggcggcggcgggggggacccggaactctgcgccacggacgagatgatccccttcaaggacgagggcgatcctcagaaggaaaagatcttcgccgagatcagtcatcccgaagaggaaggcgatttagctgacatcaagtcttccttggtgaacgagtctgaaatcatcccggccagcaacggacacgaggtggccagacaagcacaaacctctcaggagccctaccacgacaaggccagagaacaccccgatgacggaaagcatccagatggaggcctctacaacaagggaccctcctactcgagttattccgggtacataatgatgccaaatatgaataacgacccatacatgtcaaatggatctctttctccacccatcccgagaacatcaaataaagtgcccgtggtgcagccatcccatgcggtccatcctctcacccccctcatcacttacagtgacgagcacttttctccaggatcacacccgtcacacatcccatcagatgtcaactccaaacaaggcatgtccagacatcctccagctcctgatatccctactttttatcccttgtctccgggtggtgttggacagatcaccccacctcttggctggcaaggtcagcctgtatatcccatcacgggtggattcaggcaaccctacccatcctcactgtcagtcgacacttccatgtccaggttttcccatcatatgattcccggtcctcctggtccccacacaactggcatccctcatccagctattgtaacacctcaggtcaaacaggaacatccccacactgacagtgacctaatgcacgtgaagcctcagcatgaacagagaaaggagcaggagccaaaaagacctcacattaagaagcctctgaatgcttttatgttatacatgaaagaaatgagagcgaatgtcgttgctgagtgtactctaaaagaaagtgcagctatcaaccagattcttggcagaaggtggcatgccctctcccgtgaagagcaggctaaatattatgaattagcacggaaagaaagacagctacatatgcagctttatccaggctggtctgcaagagacaattatggtaagaaaaagaagaggaagagagagaaactacaggaatctgcatcaggtacaggtccaagaatgacagctgcctacatctga

SEQ ID NO: 15 - protein, 399 aa, Artificial Sequence
  Human LEF1
MPQLSGGGGGGGGDPELCATDEMIPFKDEGDPQKEKIFAEISHPEEEGDLADIKSSLVNESEIIPASNGHEVARQAQTSQEPYHDKAREHPDDGKHPDGGLYNKGPSYSSYSGYIMMPNMNNDPYMSNGSLSPPIPRTSNKVPVVQPSHAVHPLTPLITYSDEHFSPGSHPSHIPSDVNSKQGMSRHPPAPDIPTFYPLSPGGVGQITPPLGWQGQPVYPITGGFRQPYPSSLSVDTSMSRFSHHMIPGPPGPHTTGIPHPAIVTPQVKQEHPHTDSDLMHVKPQHEQRKEQEPKRPHIKKPLNAFMLYMKEMRANVVAECTLKESAAINQILGRRWHALSREEQAKYYELARKERQLHMQLYPGWSARDNYGKKKKRKREKLQESASGTGPRMTAAYI

SEQ ID NO: 16 - protein, 131 aa, Artificial Sequence
  Human FZD8 Fri domain
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTT

SEQ ID NO: 17 - protein, 227 aa, Artificial Sequence
  Human IgG1 Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 227 aa, Artificial Sequence
  Human IgG1 Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 230 aa, Artificial Sequence
  Human IgG1 Fc region
KSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 232 aa, Artificial Sequence
  Human IgG1 Fc region
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 224 aa, Artificial Sequence
  Human IgG2 Fc region
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - protein, 360 aa, Artificial Sequence
  FZD8-Fc 54F28 amino acid sequence
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLS

SEQ ID NO: 23 - protein, 390 aa, Artificial Sequence
  FZD8-Fc 54F28
  SIGNAL (1)..(27)
MEWGYLLEVTSLLAALLLLQRSPFVHAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2018/0223372 A1
(43) Pub. Date: Aug. 9, 2018

(54) IDENTIFICATION OF PREDICTIVE BIOMARKERS ASSOCIATED WITH WNT PATHWAY INHIBITORS

(71) Applicant: OncoMed Pharmaceutical, Inc., Redwood City, CA (US)

(72) Inventors: Ann M. KAPOUN, Mountain View, CA (US); Chun ZHANG, Palo Alto, CA (US)

(21) Appl. No.: 15/749,855

(22) PCT Filed: Aug. 3, 2016
(86) PCT No.: PCT/US16/45249

§ 371 Date: Feb. 2, 2018

Related U.S. Application Data
(60) Provisional application No. 62/200,164, filed on Aug. 3, 2015.

(51) Int. Cl. C12Q 1/6886; G01N 33/574; C07K 16/28; A61K 31/337; A61K 39/395

(52) U.S. Cl. CPC C12Q 1/6886; A61K 2039/505; C07K 16/2863; A61K 31/337; A61K 39/3955; G01N 2333/4706; C07K 2317/76; C07K 2317/565; C07K 2317/56; C07K 2317/24; C07K 2317/31; C07K 2317/21; C12Q 2600/106; C12Q 2600/158; G01N 33/57423

(57) ABSTRACT

The present invention provides biomarkers for identifying tumors likely to respond to treatment with Wnt pathway inhibitors. Also provided are methods for identifying tumors and/or patients that are likely to be responsive or non-responsive to treatment with a Wnt pathway inhibitor. Methods for treating a patient with cancer are provided, wherein the cancer is predicted to respond to a Wnt pathway inhibitor.

### CROSS-REFERENCE TO RELATED APPLICATIONS

This application claims the priority benefit of U.S. Provisional Application No. 62/200,164, filed Aug. 3, 2015, which is hereby incorporated by reference herein in its entirety.

### FIELD OF INVENTION

The present invention relates to the field of cancer treatment. More particularly, the invention provides methods for identifying tumors that are likely to be responsive or non-responsive to treatment with a Wnt pathway inhibitor. In addition, the invention provides methods for identifying, selecting, and/or treating patients with cancer who are likely to respond to treatment with a Wnt pathway inhibitor, either alone or in combination with other therapeutic agents.

### BACKGROUND OF THE INVENTION

Cancer is one of the leading causes of death in the developed world, with approximately 1.6 million people diagnosed with cancer and over 550,000 deaths per year in the United States alone. Overall it is estimated that more than 1 in 3 people will develop some form of cancer during their lifetime. There are more than 200 different types of cancer, four of which—breast, lung, colorectal, and prostate-account for almost half of all new cases in the United States (Siegel et al., 2012, CA: A Cancer J. for Clin., 62: 10-29).

Signaling pathways normally connect extracellular signals to the nucleus leading to the expression of genes that directly or indirectly control cell growth, differentiation, survival, and/or death. However, in a wide variety of cancers signaling pathways are dysregulated and may be linked to tumor initiation and/or progression. Signaling pathways implicated in human oncogenesis include, but are not limited to, the Wnt pathway, the Ras-Raf-MEK-ERK or MAPK pathway, the PI3K-AKT pathway, the CDKN2A/CDK4 pathway, the Bcl-2/TP53 pathway, and the NOTCH pathway.

The Wnt signaling pathway is one of several critical regulators of embryonic pattern formation, post-embryonic tissue maintenance, and stem cell biology. More specifically, Wnt signaling plays an important role in the generation of cell polarity and cell fate specification including self-renewal by stem cell populations. Unregulated activation of the Wnt pathway is associated with numerous human cancers where it is believed the activation can alter the developmental fate of cells. It is believed that the activation of the Wnt pathway may maintain tumor cells in an undifferentiated state and/or lead to uncontrolled proliferation. This may allow carcinogenesis to proceed by overtaking homeostatic mechanisms which control and/or regulate normal development and tissue repair (reviewed in Reya & Clevers, 2005, Nature, 434:843-50; Beachy et al., 2004. Nature, 432:324-31).

The Wnt signaling pathway was first elucidated in the Drosophila developmental mutant wingless (wg) and from the murine proto-oncogene int-1, now Wnt1 (Nusse & Varmus, 1982, Cell, 31:99-109; Van Ooyen & Nusse, 1984, Cell, 39:233-40: Cabrera et al., 1987, Cell, 50:659-63; Rijsewijk et al., 1987, Cell, 50:649-57). Wnt genes encode lipid-modified glycoproteins which are secreted and 19 different Wnt proteins have been identified in mammals. These secreted ligands activate a receptor complex consisting of a Frizzled (FZD) receptor family member and low-density lipoprotein (LDL) receptor-related protein 5 or 6 (LRP5/6). The FZD receptors are members of the G-protein coupled receptor (GPCR) superfamily and contain seven transmembrane domains and a large extracellular N-terminal ligand binding domain. The N-terminal ligand binding domain contains 10 conserved cysteines and is known as a cysteine-rich domain (CRD) or a “Fri domain”. There are ten human FZD receptors, FZD1, FZD2, FZD3, FZD4, FZD5, FZD6, FZD7, FZD8, FZD9, and FZD10. Different FZD CRDs have different binding affinities for specific Wnt proteins (Wu & Nusse, 2002, J. Biol. Chem., 277:41762-9). In addition, FZD receptors may be grouped into those that activate the canonical β-catenin pathway and those that activate non-canonical pathways (Miller et al., 1999, Oncogene, 18:7860-72).

A role for Wnt signaling in cancer was first uncovered with the identification of Wnt1 (originally int1) as an oncogene in mammary tumors transformed by the nearby insertion of a murine virus (Nusse & Varmus, 1982, Cell, 31:99-109). Since these early observations additional evidence for the role of Wnt signaling in breast cancer has continued to accumulate. For example, over-expression of β-catenin in the mammary glands of transgenic mice results in hyperplasias and adenocarcinomas (Imbert et al., 2001, J. Cell Biol., 153:555-68; Michaelson & Leder, 2001, Oncogene, 20:5093-9) whereas loss of Wnt signaling disrupts normal mammary gland development (Tepera et al., 2003, J. Cell Sci., 116:1137-49; Hatsell et al., 2003, J. Mammary Gland Biol. Neoplasia, 8:145-58). In human breast cancer, β-catenin accumulation implicates activated Wnt signaling in over 50% of carcinomas, and though specific mutations have not been identified, up-regulation of Frizzled receptor expression has been observed (Brennan & Brown, 2004, J. Mammary Gland Biol. Neoplasia, 9:119-31; Malovanovic et al., 2004, Int. J. Oncol., 25:1337-42). Activation of the Wnt pathway is also associated with colorectal cancer, lung cancer, pancreatic cancer, and melanoma.

Thus the Wnt pathway has been identified as a target for cancer therapy and treatment. As drug discovery and development advances, especially in the cancer field, the “one drug fits all” approach is shifting to a “personalized medicine” strategy. Personalized medicine strategies may include treatment regimens that are based upon cancer biomarkers, including prognostic biomarkers, pharmacodynamic biomarkers, and predictive biomarkers. In general, predictive biomarkers assess the likelihood that a tumor or cancer will be responsive to or sensitive to a specific therapeutic agent or a combination of therapeutic agents, and may allow for the identification and/or the selection of patients most likely to benefit from the use of that agent or agents.

### SUMMARY OF THE INVENTION

The invention provides the identification of predictive biomarkers associated with the use of Wnt pathway inhibitors in the treatment of cancer. Also provided are methods of using the predictive biomarkers for identifying, selecting, and/or classifying tumors and/or patients with cancer as likely to be responsive or non-responsive to treatment with a Wnt pathway inhibitor either as a single agent or in combination with additional therapeutics. Methods for treating patients that are predicted to be responsive to treatment with a Wnt pathway inhibitor are also provided.

In one aspect, the invention provides predictive biomarkers for identifying patients with cancer, particularly lung cancer such as non-small cell lung cancer (NSCLC), likely to respond to treatment with Wnt pathway inhibitors. Additionally provided are methods for identifying tumors and/or patients that are likely to be responsive or non-responsive to treatment with a Wnt pathway inhibitor. Further provided are methods of treating cancer, particularly lung cancer, in a patient with a Wnt pathway inhibitor, wherein the patient is predicted to be or has been identified as likely to be responsive to the Wnt pathway inhibitor. As used herein, “lung tumor” or “lung cancer”, includes non-small cell lung cancer (NSCLC) including but not limited to, adenocarcinoma, squamous cell carcinoma, large cell carcinoma, and large cell neuroendocrine carcinoma.

In one aspect, the invention provides a method of identifying a human lung tumor that is likely to be responsive or non-responsive to treatment with a Wnt pathway inhibitor, the method comprising: (a) obtaining a sample of the human lung tumor; (b) measuring the expression level of a biomarker signature in the sample, wherein the biomarker signature comprises LEF1; and (c) identifying the tumor as likely to be responsive or non-responsive to treatment based upon the expression level of the biomarker signature. In some embodiments, the method comprises identifying a human lung tumor that is likely to be responsive or non-responsive to treatment with a Wnt pathway inhibitor in combination with at least one additional therapeutic agent. In some embodiments, the method comprises identifying a human lung tumor that is likely to be responsive or non-responsive to treatment with a Wnt pathway inhibitor in combination with chemotherapy.

In another aspect, the invention provides a method of classifying a human lung tumor as likely to be responsive or non-responsive to treatment with a Wnt pathway inhibitor, the method comprising: (a) obtaining a sample of the human lung tumor; (b) measuring the expression level of a biomarker signature in the sample, wherein the biomarker signature comprises LEF1; and (c) classifying the lung tumor as likely to be responsive or non-responsive to treatment based upon the expression level of the biomarker signature. In some embodiments, the method comprises classifying a human lung tumor as likely to be responsive or non-responsive to treatment with a Wnt pathway inhibitor in combination with at least one additional therapeutic agent. In some embodiments, the method comprises classifying a human lung tumor that is likely to be responsive or non-responsive to treatment with a Wnt pathway inhibitor in combination with chemotherapy.

In another aspect, the invention provides a method of determining the responsiveness (or sensitivity) of a human lung tumor to treatment with a Wnt pathway inhibitor, the method comprising: (a) obtaining a sample of the human lung tumor; (b) measuring the expression level of a biomarker signature in the sample, wherein the biomarker signature comprises LEF1; and (c) determining the responsiveness of the lung tumor to treatment based upon the expression level of the biomarker signature. In some embodiments, the method comprises determining the responsiveness or sensitivity of a human lung tumor to treatment with a Wnt pathway inhibitor in combination with at least one additional therapeutic agent. In some embodiments, the method comprises determining the responsiveness or sensitivity of a human lung tumor to treatment with a Wnt pathway inhibitor in combination with chemotherapy.

In another aspect, the invention provides a method of identifying a patient with lung cancer who is likely to respond to treatment with a Wnt pathway inhibitor, the method comprising: (a) obtaining a sample of the patient's lung tumor; (b) measuring the expression level of a biomarker signature in the sample, wherein the biomarker signature comprises LEF1; and (c) identifying the patient who is likely to respond to treatment based upon the expression level of the biomarker signature. In some embodiments, the method comprises identifying a patient with lung cancer who is likely to respond to treatment with a Wnt pathway inhibitor in combination with at least one additional therapeutic agent. In some embodiments, the method comprises identifying a patient with lung cancer who is likely to respond to treatment with a Wnt pathway inhibitor in combination with chemotherapy.

In another aspect, the invention provides a method of selecting a patient with lung cancer for treatment with a Wnt pathway inhibitor, the method comprising: (a) obtaining a sample of the patient's lung tumor; (b) measuring the expression level of a biomarker signature in the sample, wherein the biomarker signature comprises LEF1; (c) selecting the patient for treatment based upon the expression level of the biomarker signature. In some embodiments, the method comprises selecting a patient with lung cancer for treatment with a Wnt pathway inhibitor in combination with at least one additional therapeutic agent. In some embodiments, the method comprises selecting a patient with lung cancer for treatment with a Wnt pathway inhibitor in combination with chemotherapy.

In another aspect, the invention provides a method of treating lung cancer in a patient, comprising: (a) identifying if the patient is likely to respond to treatment with a Wnt pathway inhibitor, wherein the identification comprises: (i) obtaining a sample of the patient's lung cancer; (ii) measuring the expression level of a biomarker signature in the sample, wherein the biomarker signature comprises LEF1; and (iii) identifying the patient who is likely to respond to treatment based upon the expression level of the biomarker signature; and (b) administering a therapeutically effective amount of a Wnt pathway inhibitor to the patient who is likely to response to treatment. In some embodiments, the method comprises administering to the patient the Wnt pathway inhibitor in combination with at least one additional therapeutic agent. In some embodiments, the method comprises administering to the patient the Wnt pathway inhibitor in combination with chemotherapy.

In another aspect, the invention provides a method of treating lung cancer in a patient, comprising: administering a therapeutically effective amount of a Wnt pathway inhibitor to the patient; wherein the patient is predicted to respond to treatment with the Wnt pathway inhibitor based upon the expression level of a biomarker signature in a sample of the patient's lung cancer, wherein the biomarker signature comprises LEF1. In some embodiments, the patient is predicted to respond to treatment with a Wnt pathway inhibitor in combination with at least one additional therapeutic agent. In some embodiments, the patient is predicted to respond to treatment with a Wnt pathway inhibitor in combination with chemotherapy. In some embodiments, the method comprises administering to the patient the Wnt pathway inhibitor in combination with at least one additional therapeutic agent. In some embodiments, the method comprises administering to the patient the Wnt pathway inhibitor in combination with chemotherapy.

In another aspect, the invention provides a method for increasing the likelihood of effective treatment with a Wnt pathway inhibitor, comprising: (a) identifying if a patient has a lung tumor that is likely to respond to treatment with a Wnt pathway inhibitor, wherein the identification comprises: (i) obtaining a sample of the patient's lung tumor; (ii) measuring the expression level of a biomarker signature in the sample, wherein the biomarker signature comprises LEF1; and (iii) identifying the patient who is likely to respond to treatment based upon the expression level of the biomarker signature; and (b) administering a therapeutically effective amount of the Wnt pathway inhibitor to the patient. In some embodiments, the method comprises identifying if a patient has a tumor that is likely to respond to treatment with a Wnt pathway inhibitor in combination with at least one additional therapeutic agent. In some embodiments, the method comprises identifying if a patient has a tumor that is likely to respond to treatment with a Wnt pathway inhibitor in combination with chemotherapy. In some embodiments, the method comprises administering to the patient the Wnt pathway inhibitor in combination with at least one additional therapeutic agent. In some embodiments, the method comprises administering to the patient the Wnt pathway inhibitor in combination with chemotherapy.

In another aspect, the invention provides a method for increasing the likelihood of effective treatment with a Wnt pathway inhibitor, comprising: administering a therapeutically effective amount of a Wnt pathway inhibitor to a patient; wherein the patient is identified as likely to respond to treatment with the Wnt pathway inhibitor based upon expression level of a biomarker signature in a sample of the patient's lung tumor, wherein the biomarker signature comprises LEF1. In some embodiments, the patient is identified as likely to respond to treatment with a Wnt pathway inhibitor in combination with at least one additional therapeutic agent. In some embodiments, the patient is identified as likely to respond to treatment with a Wnt pathway inhibitor in combination with chemotherapy. In some embodiments, the method comprises administering to the patient the Wnt pathway inhibitor in combination with at least one additional therapeutic agent. In some embodiments, the method comprises administering to the patient the Wnt pathway inhibitor in combination with chemotherapy.

In certain embodiments of each of the aforementioned aspects, as well as other aspects and/or embodiments described elsewhere herein, the Wnt pathway inhibitor is an antibody. In some embodiments, the Wnt pathway inhibitor is an antibody that specifically binds at least one human Frizzled (FZD) protein or fragment thereof. In some embodiments, the Wnt pathway inhibitor is an antibody that specifically binds at least one FZD protein selected from the group consisting of: FZD1, FZD2, FZD3, FZD4, FZD5, FZD6, FZD7, FZD8, FZD9, and FZD10. In some embodiments, the Wnt pathway inhibitor is an antibody that specifically binds at least one FZD protein selected from the group consisting of: FZD1, FZD2, FZD5, FZD7, and FZD8. In some embodiments, the Wnt pathway inhibitor is an antibody that specifically binds FZD1, FZD2, FZD5, FZD7, and FZD8. In certain embodiments, the Wnt pathway inhibitor is an antibody which comprises: (a) a heavy chain CDR1 comprising GFTFSHYTLS (SEQ ID NO: 1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6).

In certain embodiments, the FZD-binding agent comprises a heavy chain variable region having at least about 85%, at least about 90%, at least about 95%, at least about 97%, or at least about 99% sequence identity to SEQ ID NO:7. In certain embodiments, the FZD-binding agent comprises a light chain variable region having at least about 85%, at least about 90%, at least about 95%, at least about 97%, or at least about 99% sequence identity to SEQ ID NO:8. In certain embodiments, the Wnt pathway inhibitor is an antibody which comprises a heavy chain variable region comprising SEQ ID NO:7 and a light chain variable region comprising SEQ ID NO:8. In certain embodiments, the Wnt pathway inhibitor is an antibody which comprises a heavy chain variable region and a light chain variable region encoded by the plasmid deposited with ATCC as PTA-9541. In certain embodiments, the Wnt pathway inhibitor is an antibody which comprises a heavy chain and a light chain encoded by the plasmid deposited with ATCC as PTA-9541. In some embodiments, the Wnt pathway inhibitor is OMP-18R5 (also known as 18R5 or vantictumab).

In certain embodiments of each of the aforementioned aspects, as well as other aspects and/or embodiments described elsewhere herein, the Wnt pathway inhibitor is a soluble receptor. In some embodiments, the Wnt pathway inhibitor comprises the extracellular domain of a FZD receptor protein. In some embodiments, the Wnt pathway inhibitor comprises a Fri domain of a FZD protein. In some embodiments, the Wnt pathway inhibitor comprises the Fri domain of FZD8. In certain embodiments, the Wnt pathway inhibitor comprises the Fri domain of FZD8 and a human Fc domain. In some embodiments, the Wnt pathway inhibitor is the soluble receptor OMP-54F28 (also known as 54F28 or ipafricept).

In some embodiments, the method further comprises administering at least one additional therapeutic agent to the patient. In some embodiments, the at least one additional therapeutic agent is a chemotherapeutic agent. In some embodiments, the additional therapeutic agent is a taxane. In some embodiments, the additional therapeutic agent is paclitaxel or a derivative thereof. In some embodiments, the additional therapeutic agent is docetaxel or a derivative thereof. In some embodiments, the additional therapeutic agent is a platinum complex, such as but not limited to, cisplatin or carboplatin. In some embodiments, the additional therapeutic agent is pemetrexed. In some embodiments, the additional therapeutic agent is gemcitabine. In some embodiments, the additional therapeutic agent is irinotecan. In some embodiments, the additional therapeutic agents are cisplatin or carboplatin and pemetrexed. In some embodiments, the additional therapeutic agents are cisplatin or carboplatin and a taxane. In some embodiments, the additional therapeutic agents are cisplatin or carboplatin and paclitaxel. In some embodiments, the additional therapeutic agents are cisplatin or carboplatin and docetaxel. In some embodiments, the at least one additional therapeutic agent is a tyrosine kinase inhibitor (TKI). In some embodiments, the at least one additional therapeutic agent is an EGFR inhibitor. In some embodiments, the additional therapeutic agent is erlotinib (TARCEVA) or gefitinib (IRESSA). In some embodiments, the additional therapeutic agent is an anti-VEGFR antibody. In some embodiments, the additional therapeutic agent is bevacizumab. In some embodiments, the additional therapeutic agent is an immune checkpoint inhibitor. In some embodiments, the additional therapeutic agent is an anti-PD-1 antibody, an anti-PD-L1 antibody, or an anti-CTLA-4 antibody.

In certain embodiments of each of the aforementioned aspects, as well as other aspects and/or embodiments described elsewhere herein, a sample includes, but is not limited to, any clinically relevant tissue sample, such as a tumor biopsy, a core biopsy tissue sample, a fine needle aspirate, a hair follicle, or a sample of bodily fluid, such as blood, plasma, serum, lymph, ascites fluid, cystic fluid, or urine. In some embodiments, the sample is taken from a patient having a tumor or cancer. In some embodiments, the sample is a primary tumor. In some embodiments, the sample is a metastasis. In some embodiments, the sample is a tissue sample. In some embodiments, the sample is a tumor sample. In some embodiments, the sample is a fresh tissue sample. In some embodiments, the sample is a frozen tissue sample. In some embodiments, the sample is a fresh frozen (FF) tissue sample. In some embodiments, the sample is a formalin-fixed paraffin embedded (FFPE) tissue sample. In some embodiments, the sample is whole blood, plasma, or serum. In some embodiments, the sample is cells. In some embodiments, the sample is circulating tumor cells (CTCs).

In certain embodiments of each of the aforementioned aspects, as well as other aspects and/or embodiments described elsewhere herein, the expression level of a biomarker is determined using methods that detect the expression level of nucleic acids (e.g., DNA or RNA). In some embodiments, the expression level of a biomarker is determined using PCR-based methods, such as but not limited to, reverse transcription PCR (RT-PCR), quantitative RT-PCR (qPCR), TaqMan™, or TaqMan™ low density array (TLDA). In some embodiments, the expression level of a biomarker is determined using a microarray. In some embodiments, the expression level of a biomarker is determined using a sequencing method, such as but not limited to, next-generation sequencing (NGS). RNA sequencing (RNA-Seq), and whole transcriptome shotgun sequencing (WTSS). NGS is rapidly becoming the method of choice for transcriptional profiling experiments. In contrast to microarray technology, high throughput sequencing allows identification of novel transcripts, does not require a sequenced genome, and circumvents background noise associated with fluorescence quantification. Furthermore, unlike hybridization-based detection, NGS allows genome-wide analysis of transcription at single nucleotide resolution, including identification of alternative splicing events and post-transcriptional RNA editing events.

In some embodiments, the expression level of a biomarker is measured or determined by a PCR-based assay. In some embodiments, an assay uses one or more primer pairs and probes specific for amplification of LEF1 RNA. In some embodiments, an assay uses one or more primer pairs and probes specific for amplification of LEF1 mRNA. In some embodiments, the primer pairs are a forward (sense) primer and a reverse (anti-sense) primer that consist essentially of at least eight contiguous nucleotides of SEQ ID NO: 14. In some embodiments, a forward and reverse primer pair hybridizes to a nucleotide sequence of SEQ ID NO: 14.

In certain embodiments of each of the aforementioned aspects, as well as other aspects and/or embodiments described elsewhere herein, the expression level of a biomarker is determined using methods that detect the expression level of a protein. In some embodiments, the expression level of a biomarker is measured or determined by multi-analyte profile testing, radioimmunoassay (RIA), Western blot assay, immunofluorescent assay, enzyme immunoassay, enzyme linked immunosorbent assay (ELISA), immunoprecipitation assay, chemiluminescent assay, immunohistochemical assay (IHC), dot blot assay, or slot blot assay. In some embodiments wherein the assay uses an antibody, the antibody is detectably labeled. In some embodiments, the label is selected from the group consisting of an immunofluorescent label, a chemiluminescent label, a phosphorescent label, an enzyme label, a radiolabel, an avidin/biotin label, colloidal gold particles, colored particles, and magnetic particles.

In some embodiments, the method used to detect and/or determine LEF1 expression in a tumor sample is an immunohistochemistry (IHC) assay. In some embodiments, the method used to detect and/or determine LEF1 expression in a tumor sample comprises an H-score evaluation.

The invention also provides a kit comprising a container, wherein the container contains at least one reagent for specifically detecting the expression of a biomarker of the invention. In certain embodiments, the reagent is an antibody or nucleic acid probe that binds a biomarker of the invention. In some embodiments, a kit comprises a forward primer that hybridizes to SEQ ID NO: 14, a reverse primer that hybridizes to SEQ ID NO: 14, and a probe. In some embodiments, a kit comprises an anti-LEF1 antibody.

Where aspects or embodiments of the invention are described in terms of a Markush group or other grouping of alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### BRIEF DESCRIPTIONS OF THE DRAWINGS

FIGS. 1A-1G. Classification of lung tumors as responsive or non-responsive to treatment with anti-FZD antibody OMP-18R5. FIG. 1A—Lung tumor OMP-LU02. FIG. 1B—Lung tumor OMP-LU11. FIG. 1C—Lung tumor OMP-LU24. FIG. 1D—Lung tumor OMP-LU25. FIG. 1E—Lung tumor OMP-LU33. FIG. 1F—Lung tumor OMP-LU45. FIG. 1G—Lung tumor OMP-LU56. For each experiment, tumors cells were injected subcutaneously into NOD/SCID mice and allowed to grow to a specific size. Mice were treated with OMP-18R5 (-▪-) or a control antibody (-●-). Data is shown as tumor volume (mm³) over days post-treatment.

FIG. 2. Performance curves for the top 10 ranked genes.

FIG. 3. Distribution of LEF1 gene expression in lung tumors that are non-responders (NR) and responders (R) to treatment with anti-FZD antibody OMP-18R5.

FIG. 4. Correlation of LEF1 gene expression with ratio of tumor volume (RTV). RTV=100−tumor volume of OMP-18R5-treated/tumor volume of control.

FIGS. 5A-5B. Classification of lung tumors as responsive or non-responsive to treatment with anti-FZD antibody OMP-18R5. FIG. 5A—Lung tumor OMP-LU52. FIG. 5B—Lung tumor OMP-LU53. For each experiment, tumors cells were injected subcutaneously into NOD/SCID mice and allowed to grow to a specific size. Mice were treated with OMP-18R5 (-▪-) or a control antibody (-●-). Data is shown as tumor volume (mm³) over days post-treatment.

FIGS. 6A-6L. Classification of lung tumors as responsive or non-responsive to treatment with OMP-18R5 in combination with paclitaxel. FIG. 6A—Lung tumor OMP-LU02. FIG. 6B—Lung tumor OMP-LU11. FIG. 6C—Lung tumor OMP-LU24. FIG. 6D—Lung tumor OMP-LU25. FIG. 6E—Lung tumor OMP-LU42. FIG. 6F—Lung tumor OMP-LU52. FIG. 6G—Lung tumor OMP-LU53. FIG. 6H—Lung tumor OMP-LU56. FIG. 6I—Lung tumor OMP-LU63. FIG. 6J—Lung tumor OMP-LU77. FIG. 6K—Lung tumor OMP-LU104. FIG. 6L—Lung tumor OMP-LU121. For each experiment, tumors cells were injected subcutaneously into NOD/SCID mice and allowed to grow to a specific size. Mice were treated OMP-18R5 (-▪-), paclitaxel (-ϵ-), a combination of OMP-18R5 and paclitaxel (—⋄—), or a control antibody (-●-). Data is shown as tumor volume (mm³) over days post-treatment.

FIGS. 7A and 7B. Distribution of LEF1 gene expression in lung tumors that are non-responders (NR) and responders (R) to treatment with OMP-18R5 in combination with paclitaxel. Lung tumors treated with OMP-18R5 and paclitaxel on the same day (-●-) or lung tumors treated with OMP-18R5 and paclitaxel sequentially (-▴-). FIG. 7A—Analysis of results. FIG. 7B—Analysis after reclassification of tumor OMP-LU121.

FIG. 8. Population prevalence estimation of the LEF1 biomarker in three public datasets.

### DETAILED DESCRIPTION OF THE INVENTION

### I. Definitions

To facilitate an understanding of the present invention, a number of terms and phrases are defined below.

The term “biomarker” as used herein may include but is not limited to, nucleic acids and proteins, and variants and fragments thereof. A biomarker may be DNA comprising the entire or partial nucleic acid sequence encoding the biomarker, or the complement of such a sequence. Biomarker nucleic acids useful in the invention are considered to include both DNA and RNA comprising the entire or partial sequence of any of the nucleic acid sequences of interest. Biomarker proteins are considered to comprise the entire or partial amino acid sequence of any of the biomarker proteins or polypeptides.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen-binding site. As used herein, the term encompasses intact polyclonal antibodies, intact monoclonal antibodies, single chain antibodies, antibody fragments (such as Fab, Fab′, F(ab′)2, and Fv fragments), single chain Fv (scFv) antibodies, multispecific antibodies such as bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological binding activity. An antibody can be any of the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises at least one antigen-binding site or epitope-binding site.

The term “variable region” of an antibody refers to the variable region of an antibody light chain, or the variable region of an antibody heavy chain, either alone or in combination. The variable region of a heavy chain or a light chain generally consists of four framework regions (FR) connected by three complementarity determining regions (CDRs), also known as “hypervariable regions”. The CDRs in each chain are held together in close proximity by the framework regions and contribute to the formation of the antigen-binding site(s) of the antibody. There are at least two techniques for determining CDRs: (1) an approach based on cross-species sequence variability (i.e., Kabat et al., 1991, Sequences of Proteins of Immunological Interest. 5th Edition, National Institutes of Health, Bethesda, Md.), and (2) an approach based on crystallographic studies of antigen-antibody complexes (Al-Lazikani et al., 1997, J. Mol. Biol., 273:927-948). In addition, combinations of these two approaches are sometimes used in the art to determine CDRs.

The term “monoclonal antibody” as used herein refers to a homogeneous antibody population involved in the highly specific recognition and binding of a single antigenic determinant or epitope. This is in contrast to polyclonal antibodies that typically include a mixture of different antibodies directed against a variety of different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody portion, and any other modified immunoglobulin molecule comprising an antigen-binding site. Furthermore, “monoclonal antibody” refers to such antibodies made by any number of techniques, including but not limited to, hybridoma production, phage selection, recombinant expression, and transgenic animals.

The term “humanized antibody” as used herein refers to antibodies that are specific immunoglobulin chains, chimeric immunoglobulins, or fragments thereof that contain minimal non-human sequences.

The term “human antibody” as used herein refers to an antibody produced by a human or an antibody having an amino acid sequence corresponding to an antibody produced by a human.

The term “chimeric antibody” as used herein refers to an antibody wherein the amino acid sequence of the immunoglobulin molecule is derived from two or more species. Typically, the variable region of the light chain and the variable region of the heavy chain correspond to the variable regions of an antibody derived from one species of mammals (e.g., mouse, rat, rabbit, etc.) with the desired specificity, affinity, and/or binding capability, while the constant regions correspond to sequences from an antibody derived from another species (e.g., human).

The term “affinity-matured antibody” as used herein refers to an antibody with one or more alterations in one or more CDRs thereof that result in an improvement in the affinity of the antibody for antigen, compared to a parent antibody that does not possess those alterations(s). The definition also includes alterations in non-CDR residues made in conjunction with alterations to CDR residues. Generally, affinity-matured antibodies will have nanomolar or even picomolar affinities for the target antigen.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5 or 8-10 amino acids in a unique spatial conformation.

The terms “selectively binds” or “specifically binds” mean that a binding agent or an antibody reacts or associates more frequently, more rapidly, with greater duration, with greater affinity, or with some combination of the above to the epitope, protein, or target molecule than with alternative substances, including unrelated or related proteins. In certain embodiments “specifically binds” means, for instance, that a binding agent binds a protein with a K_{D} of about 0.1 mM or less, but more usually less than about 1 μM. In certain embodiments, “specifically binds” means that a binding agent binds a target at times with a K_{D} of at least about 0.1 μM or less, at other times at least about 0.01 μM or less, and at other times at least about 1 nM or less. Because of the sequence identity between homologous proteins in different species, specific binding can include a binding agent that recognizes a protein in more than one species (e.g., a human FZD protein and a mouse FZD protein). Likewise, because of homology within certain regions of polypeptide sequences of different proteins, particularly proteins within a protein family, specific binding can include a binding agent that recognizes more than one protein (e.g., human FZD1 and human FZD7). It is understood that, in certain embodiments, a binding agent that specifically binds a first target may or may not specifically bind a second target. As such, “specific binding” does not necessarily require (although it can include) exclusive binding, i.e. binding to a single target. Thus, a binding agent may, in certain embodiments, specifically bind more than one target. In certain embodiments, multiple targets may be bound by the same binding site on the binding agent. For example, a binding agent may, in certain instances, comprise two identical binding sites, each of which specifically binds the same target on two or more proteins. In certain alternative embodiments, a binding agent may be bispecific or multispecific and comprise at least two binding sites with differing specificities. By way of non-limiting example, a bispecific agent may comprise one binding site that recognizes an epitope on one protein (e.g., a human FZD) and further comprise a second, different binding site that recognizes a different epitope on a second protein (e.g., a human Wnt protein). Generally, but not necessarily, reference to binding means specific binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention, for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies or fusion proteins, in certain embodiments, the polypeptides can occur as single chains or associated chains (e.g., dimers).

The terms “polynucleotide” and “nucleic acid” and “nucleic acid molecule” are used interchangeably herein and refer to polymers of nucleotides of any length, and include DNA and RNA. The polynucleotides can be deoxyribonucleotides, ribonucleotides, modified nucleotides or bases, and/or their analogs, or any substrate that can be incorporated into a polymer by DNA or RNA polymerase.

“Conditions of high stringency” may be identified by conditions that: (1) employ low ionic strength and high temperature for washing, for example 15 mM sodium chloride/1.5 mM sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 in 5×SSC (0.75M NaCl, 75 mM sodium citrate) at 42° C.; or (3) employ during hybridization 50% formamide in 5×SSC, 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 50% formamide, followed by a wash consisting of 0.1×SSC containing EDTA at 55° C.

The terms “identical” or percent “identity” in the context of two or more nucleic acids or two or more polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BcstFit, GCG Wisconsin Package, and variations thereof. In some embodiments, two nucleic acids or polypeptides of the invention are substantially identical, meaning they have at least about 70%, at least about 75%, at least about 80%, at least about 85%, at least about 90%, and in some embodiments at least about 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 nucleotides or amino acid residues, at least about 60-80 nucleotides or amino acid residues in length or any integral value therebetween. In some embodiments, identity exists over a longer region than 60-80 nucleotides or amino acid residues, such as at least about 80-100 nucleotides or amino acid residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Preferably, conservative substitutions in the sequences of the polypeptides and antibodies of the invention do not abrogate the binding of the polypeptide or antibody containing the amino acid sequence, to the antigen to which the polypeptide or antibody binds. Methods of identifying nucleotide and amino acid conservative substitutions which do not eliminate antigen binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (non-cancerous) or malignant (cancerous) including pre-cancerous lesions.

The term “metastasis” as used herein refers to the process by which a cancer spreads or transfers from the site of origin to other regions of the body with the development of a similar cancerous lesion at a new location. A “metastatic” or “metastasizing” cell is one that loses adhesive contacts with neighboring cells and migrates (e.g., via the bloodstream or lymph) from the primary site of disease to secondary sites.

The terms “cancer stem cell” and “CSC” and “tumor stem cell” and “tumor initiating cell” are used interchangeably herein and refer to cells from a cancer or tumor that: (1) have extensive proliferative capacity; 2) are capable of asymmetric cell division to generate one or more types of differentiated cell progeny wherein the differentiated cells have reduced and/or limited proliferative or developmental potential; and (3) are capable of symmetric cell divisions for self-renewal or self-maintenance. These properties confer on the cancer stem cells the ability to form or establish a tumor or cancer upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a random sample of cells from the tumor to form palpable tumors upon serial transplantation into immunocompromised hosts (e.g., mice). This definition also includes enriched and/or isolated populations of cancer stem cells that form palpable tumors upon serial transplantation into immunocompromised hosts (e.g., mice).

The term “patient” refers to any animal (e.g., a mammal), including, but not limited to, humans, non-human primates, canines, felines, rodents, and the like, which is to be the recipient of a particular treatment. The terms “patient” and “subject” are used interchangeably herein. Typically, the terms “patient” and “subject” are used in reference to a human patient.

The term “pharmaceutically acceptable” refers to a product or compound approved (or approvable) by a regulatory agency of the Federal government or a state government or listed in the U.S. Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier, or adjuvant that can be administered to a subject, together with at least one agent (e.g., an antibody) of the present disclosure, and which does not destroy the activity of the agent. The excipient, carrier, or adjuvant should be non-toxic when administered with an agent in doses sufficient to deliver a therapeutic effect.

The terms “effective amount” or “therapeutically effective amount” or “therapeutic effect” refer to an amount of a binding agent, an antibody, polypeptide, polynucleotide, small organic molecule, or other drug effective to “treat” a disease or disorder in a subject or mammal. In the case of cancer, the therapeutically effective amount of an agent (e.g., an antibody) has a therapeutic effect and as such can reduce the number of cancer cells; decrease tumorigenicity, tumorigenic frequency, or tumorigenic capacity; reduce the number or frequency of cancer stem cells; reduce the tumor size; reduce the cancer cell population; inhibit and/or stop cancer cell infiltration into peripheral organs including, for example, the spread of cancer into soft tissue and bone; inhibit and/or stop tumor or cancer cell metastasis; inhibit and/or stop tumor or cancer cell growth; relieve to some extent one or more of the symptoms associated with the cancer; reduce morbidity and mortality; improve quality of life; or a combination of such effects. To the extent the agent (e.g., an antibody) prevents growth and/or kills existing cancer cells, it can be referred to as cytostatic and/or cytotoxic.

The terms “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus those in need of treatment include those already diagnosed with the disorder; those prone to have the disorder; and those in whom the disorder is to be prevented. In some embodiments, a subject is successfully “treated” according to the methods of the present invention if the patient shows one or more of the following: a reduction in the number of and/or complete absence of cancer cells; a reduction in the tumor size; an inhibition of tumor growth; inhibition of and/or an absence of cancer cell infiltration into peripheral organs including the spread of cancer cells into soft tissue and bone; inhibition of and/or an absence of tumor or cancer cell metastasis; inhibition and/or an absence of cancer growth; relief of one or more symptoms associated with the specific cancer; reduced morbidity and mortality; improvement in quality of life; reduction in tumorigenicity; reduction in the number or frequency of cancer stem cells; or some combination of such effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Methods of Use of Predictive Biomarkers

In some aspects of the invention described herein, the responsiveness or sensitivity of human lung tumors to an anti-FZD antibody significantly correlated with increased LEF1 expression at both the gene and protein levels. The correlation between high levels of LEF1 expression and the responsiveness of lung tumors to the anti-FZD antibody OMP-18R5 can be exploited to improve methods of treating lung cancer. Selecting lung cancer patients for treatment comprising the anti-FZD antibody OMP-18R5 whose tumors are determined to likely be responsive to treatment based on the LEF1 expression level should increase overall therapeutic value. In some embodiments, therapeutic efficacy can be improved by not selecting lung cancer patients for treatment comprising OMP-18R5 whose tumors are determined to likely be non-responsive to treatment.

LEF1 (lymphoid enhancer factor-1) is a member of the LEF-1/TCF family of transcription factors. These proteins were originally identified as lymphoid-specific DNA-binding proteins, and were subsequently shown to interact with β-catenin and be involved in Wnt pathway signaling. The nucleotide and amino acid sequences of human LEF1 (UniProtKB No. Q9UJU2) are well-known and disclosed herein as SEQ ID NO: 14 and SEQ ID NO: 15, respectively.

Provided herein are methods for identifying, classifying, and/or selecting lung tumors that are likely to be responsive (“sensitive”) or non-responsive (“resistant”) to treatment with a Wnt pathway inhibitor. Provided herein are methods for identifying, classifying, and/or selecting patients that have a lung tumor or have lung cancer that are likely to be responsive (“sensitive”) or non-responsive (“resistant”) to treatment with a Wnt pathway inhibitor. In addition, provided are methods for treating patients with lung cancer who are likely to respond to treatment, are predicted to respond to treatment, and/or have been identified to respond to treatment with a Wnt pathway inhibitor.

In some embodiments, a method of identifying a human lung tumor that is likely to be responsive or non-responsive to treatment with a Wnt pathway inhibitor comprises: (a) obtaining a sample of the lung tumor; (b) measuring the expression level of a biomarker signature in the sample, wherein the biomarker signature comprises LEF1; and (c) identifying the lung tumor as likely to be responsive or non-responsive to treatment based upon the expression level of the biomarker signature. In some embodiments, a method of identifying a human lung tumor that is likely to be responsive or non-responsive to treatment with a Wnt pathway inhibitor comprises: (a) obtaining a sample of the lung tumor; (b) measuring the expression level of a biomarker signature in the sample, wherein the biomarker signature is LEF1; and (c) identifying the lung tumor as likely to be responsive or non-responsive to treatment based upon the expression level of the biomarker signature. In some embodiments, the method comprises identifying a human lung tumor that is likely to be responsive or non-responsive to treatment with a Wnt pathway inhibitor in combination with at least one additional therapeutic agent. In some embodiments, the method comprises identifying a human lung tumor that is likely to be responsive or non-responsive to treatment with a Wnt pathway inhibitor in combination with chemotherapy. In some embodiments, the method comprises identifying a human lung tumor that is likely to be responsive or non-responsive to treatment with a Wnt pathway inhibitor, wherein the Wnt pathway inhibitor is anti-FZD antibody OMP-18R5.

In some embodiments, a method of classifying a human lung tumor as likely to be responsive or non-responsive to treatment with a Wnt pathway inhibitor comprises: (a) obtaining a sample of the lung tumor; (b) measuring the expression level of a biomarker signature in the sample, wherein the biomarker signature comprises LEF1; and (c) classifying the lung tumor as likely to be responsive or non-responsive to treatment based upon the expression level of the biomarker signature. In some embodiments, a method of classifying a human lung tumor as likely to be responsive or non-responsive to treatment with a Wnt pathway inhibitor comprises: (a) obtaining a sample of the lung tumor; (b) measuring the expression level of a biomarker signature in the sample, wherein the biomarker signature is LEF1; and (c) classifying the lung tumor as likely to be responsive or non-responsive to treatment based upon the expression level of the biomarker signature. In some embodiments, the method comprises classifying a human lung tumor that is likely to be responsive or non-responsive to treatment with a Wnt pathway inhibitor in combination with at least one additional therapeutic agent. In some embodiments, the method comprises classifying a human lung tumor that is likely to be responsive or non-responsive to treatment with a Wnt pathway inhibitor in combination with chemotherapy. In some embodiments, the method comprises classifying a human lung tumor that is likely to be responsive or non-responsive to treatment with a Wnt pathway inhibitor, wherein the Wnt pathway inhibitor is anti-FZD antibody OMP-18R5.

In some embodiments, a method of determining the responsiveness (or sensitivity) of a human lung tumor to treatment with a Wnt pathway inhibitor comprises: (a) obtaining a sample of the lung tumor; (b) measuring the expression level of a biomarker signature in the sample, wherein the biomarker signature comprises LEF1; and (c) determining the responsiveness of the lung tumor to treatment based upon the expression level of the biomarker signature. In some embodiments, a method of determining the responsiveness (or sensitivity) of a human lung tumor to treatment with a Wnt pathway inhibitor comprises: (a) obtaining a sample of the lung tumor; (b) measuring the expression level of a biomarker signature in the sample, wherein the biomarker signature is LEF1; and (c) determining the responsiveness of the lung tumor to treatment based upon the expression level of the biomarker signature. In some embodiments, the method comprises determining the responsiveness of a human lung tumor to treatment with a Wnt pathway inhibitor in combination with at least one additional therapeutic agent. In some embodiments, the method comprises determining the responsiveness of a human lung tumor to treatment with a Wnt pathway inhibitor in combination with chemotherapy. In some embodiments, the method comprises determining the responsiveness of a human lung tumor to treatment with a Wnt pathway inhibitor, wherein the Wnt pathway inhibitor is anti-FZD antibody OMP-18R5.

In some embodiments, a method of identifying a patient with lung cancer who is likely to respond to treatment with a Wnt pathway inhibitor comprises: (a) obtaining a sample of the patient's lung tumor; (b) measuring the expression level of a biomarker signature in the sample, wherein the biomarker signature comprises LEF1; and (c) identifying the patient who is likely to respond to treatment based upon the expression level of the biomarker signature. In some embodiments, a method of identifying a patient with lung cancer who is likely to respond to treatment with a Wnt pathway inhibitor comprises: (a) obtaining a sample of the patient's lung tumor; (b) measuring the expression level of a biomarker signature in the sample, wherein the biomarker signature is LEF1; and (c) identifying the patient who is likely to respond to treatment based upon the expression level of the biomarker signature. In some embodiments, the method further comprises selecting the patient for treatment. In some embodiments, the method further comprises administering a therapeutically effective amount of the Wnt pathway inhibitor to the patient. In some embodiments, the method comprises identifying a patient with lung cancer who is likely to be responsive or non-responsive to treatment with a Wnt pathway inhibitor in combination with at least one additional therapeutic agent. In some embodiments, the method comprises identifying a patient with lung cancer who is likely to be responsive or non-responsive to treatment with a Wnt pathway inhibitor in combination with chemotherapy. In some embodiments, the method comprises identifying a patient with lung cancer who is likely to be responsive or non-responsive to treatment with a Wnt pathway inhibitor, wherein the Wnt pathway inhibitor is anti-FZD antibody OMP-18R5. In some embodiments, the method comprises administering a therapeutically effective amount of OMP-18R5 to the patient. In some embodiments, the method comprises administering a therapeutically effective amount of OMP-18R5 in combination with at least one additional therapeutic agent.

In some embodiments, a method of selecting a patient with lung cancer for treatment with a Wnt pathway inhibitor comprises: (a) obtaining a sample of the patient's lung tumor; (b) measuring the expression level of a biomarker signature in the sample, wherein the biomarker signature comprises LEF1; and (c) selecting the patient for treatment based upon the expression level of the biomarker signature. In some embodiments, a method of selecting a patient with lung cancer for treatment with a Wnt pathway inhibitor comprises: (a) obtaining a sample of the patient's lung tumor; (b) measuring the expression level of a biomarker signature in the sample, wherein the biomarker signature is LEF1; and (c) selecting the patient for treatment based upon the expression level of the biomarker signature. In some embodiments, the method further comprises administering a therapeutically effective amount of the Wnt pathway inhibitor to the patient. In some embodiments, the method comprises selecting a patient with lung cancer who is likely to be responsive or non-responsive to treatment with a Wnt pathway inhibitor in combination with at least one additional therapeutic agent. In some embodiments, the method comprises selecting a patient with lung cancer who is likely to be responsive or non-responsive to treatment with a Wnt pathway inhibitor in combination with chemotherapy. In some embodiments, the method comprises selecting a patient with lung cancer who is likely to be responsive or non-responsive to treatment with a Wnt pathway inhibitor, wherein the Wnt pathway inhibitor is anti-FZD antibody OMP-18R5. In some embodiments, the method comprises administering a therapeutically effective amount of OMP-18R5 to the patient. In some embodiments, the method comprises administering a therapeutically effective amount of OMP-18R5 in combination with at least one additional therapeutic agent.

In some embodiments, a method of treating lung cancer in a patient comprises: (a) identifying if the patient is likely to respond to treatment with a Wnt pathway inhibitor, wherein the identification comprises: (i) obtaining a sample of the patient's lung cancer; (ii) measuring the expression level of each biomarker of a biomarker signature in the sample, wherein the biomarker signature comprises LEF1; and (iii) identifying the patient who is likely to respond to treatment based upon the expression level of the biomarker signature; and (b) administering to the patient who is likely to respond to treatment a therapeutically effective amount of the Wnt pathway inhibitor. In some embodiments, a method of treating lung cancer in a patient comprises: (a) identifying if the patient is likely to respond to treatment with a Wnt pathway inhibitor, wherein the identification comprises: (i) obtaining a sample of the patient's lung cancer; (ii) measuring the expression level of a biomarker signature in the sample, wherein the biomarker signature is LEF1; and (iii) identifying the patient who is likely to respond to treatment based upon the expression level of the biomarker signature; and (b) administering to the patient who is likely to response to treatment a therapeutically effective amount of the Wnt pathway inhibitor. In some embodiments, the method of treating lung cancer in a patient comprises administering to the patient who is likely to respond to treatment a therapeutically effective amount of the Wnt pathway inhibitor in combination with at least one additional therapeutic agent. In some embodiments, the method of treating lung cancer in a patient comprises administering to the patient who is likely to respond to treatment a therapeutically effective amount of the Wnt pathway inhibitor in combination with chemotherapy. In some embodiments, the method of treating lung cancer in a patient comprises administering to the patient who is likely to respond to treatment a therapeutically effective amount of the Wnt pathway inhibitor, wherein the Wnt pathway inhibitor is anti-FZD antibody OMP-18R5.

In some embodiments, a method of treating lung cancer in a patient comprises: administering a therapeutically effective amount of a Wnt pathway inhibitor to the patient; wherein the patient is predicted to respond to treatment with a Wnt pathway inhibitor based upon expression levels of a biomarker signature in a sample of the patient's lung cancer, wherein the biomarker signature comprises LEF1. In some embodiments, a method of treating lung cancer in a patient comprises: administering a therapeutically effective amount of a Wnt pathway inhibitor to the patient; wherein the patient is predicted to respond to treatment with a Wnt pathway inhibitor based upon expression levels of a biomarker signature in a sample of the patient's lung cancer, wherein the biomarker signature is LEF1. In some embodiments, the method of treating lung cancer in a patient comprises administering a therapeutically effective amount of the Wnt pathway inhibitor in combination with at least one additional therapeutic agent. In some embodiments, the method of treating lung cancer in a patient comprises administering a therapeutically effective amount of the Wnt pathway inhibitor in combination with chemotherapy. In some embodiments, the method of treating lung cancer in a patient comprises administering a therapeutically effective amount of the Wnt pathway inhibitor, wherein the Wnt pathway inhibitor is anti-FZD antibody OMP-18R5.

In some embodiments, a method for increasing the likelihood of effective treatment with a Wnt pathway inhibitor comprises: (a) identifying if a patient has lung cancer that is likely to respond to treatment with a Wnt pathway inhibitor, wherein the identification comprises: (i) obtaining a sample of the patient's lung cancer; (ii) measuring the expression level of a biomarker signature in the sample of the patient's lung cancer, wherein the biomarker signature comprises LEF1; and (iii) identifying the patient who is likely to respond to treatment based upon the expression level of the biomarker signature; and (b) administering a therapeutically effective amount of the Wnt pathway inhibitor to the patient. In some embodiments, a method for increasing the likelihood of effective treatment with a Wnt pathway inhibitor comprises: (a) identifying if a patient has lung cancer that is likely to respond to treatment with a Wnt pathway inhibitor, wherein the identification comprises: (i) obtaining a sample of the patient's lung cancer; (ii) measuring the expression level of a biomarker signature in the sample of the patient's lung cancer, wherein the biomarker signature is LEF1; and (iii) identifying the patient who is likely to respond to treatment based upon the expression level of the biomarker signature; and (b) administering a therapeutically effective amount of the Wnt pathway inhibitor to the patient. In some embodiments, the method comprises administering a therapeutically effective amount of the Wnt pathway inhibitor to the patient in combination with at least one additional therapeutic agent. In some embodiments, the method comprises administering a therapeutically effective amount of the Wnt pathway inhibitor to the patient in combination with chemotherapy. In some embodiments, the method comprises administering a therapeutically effective amount of the Wnt pathway inhibitor to the patient, wherein the Wnt pathway inhibitor is anti-FZD antibody OMP-18R5.

In some embodiments, a method for increasing the likelihood of effective treatment with a Wnt pathway inhibitor comprises: administering a therapeutically effective amount of a Wnt pathway inhibitor to a patient with lung cancer; wherein the patient is identified as likely to respond to treatment with a Wnt pathway inhibitor based upon expression levels of a biomarker signature in a sample of the patient's lung cancer, wherein the biomarker signature comprises LEF1. In some embodiments, a method for increasing the likelihood of effective treatment with a Wnt pathway inhibitor comprises: administering a therapeutically effective amount of a Wnt pathway inhibitor to a patient with lung cancer; wherein the patient is identified as likely to respond to treatment with a Wnt pathway inhibitor based upon expression levels of a biomarker signature in a sample of the patient's lung cancer, wherein the biomarker signature is LEF1. In some embodiments, the patient is identified as likely to respond to treatment with a Wnt pathway inhibitor in combination with at least one additional therapeutic agent. In some embodiments, the patient is identified as likely to respond to treatment with a Wnt pathway inhibitor in combination with chemotherapy. In some embodiments, the method comprises administering the Wnt pathway inhibitor in combination with at least one additional therapeutic agent. In some embodiments, the method comprises administering the Wnt pathway inhibitor in combination with chemotherapy. In some embodiments, the method comprises administering the Wnt pathway inhibitor, wherein the Wnt pathway inhibitor is anti-FZD antibody OMP-18R5.

In some embodiments of the methods described herein, the biomarker signature comprises the biomarker LEF1. In some embodiments, the biomarker signature consists of LEF1.

In some embodiments, the biomarker signature comprises one or more additional biomarkers, in addition to LEF1. In some embodiments, the biomarker signature comprises one or more additional biomarkers selected from the group consisting of: FZD1, FZD4, FZD7, FZD8, SENP2, TCF7L1, WNT1, WNT3, and RHOU in addition to LEF1. In some embodiments, the biomarker signature comprises one or more additional biomarkers selected from the group consisting of: FZD1, FZD4, FZD7, FZD8, SENP2, TCF7L1, WNT1, WNT3, WNT11, WIF1, PYGO1, CTBP2, and RHOU in addition to LEF1.

In some embodiments for the methods described herein, the lung tumor is identified or classified as likely to be responsive or responsive to treatment with a Wnt pathway inhibitor when the expression level of LEF1 is determined to be high. In some embodiments for the methods described herein, the lung tumor is identified or classified as likely to be responsive or responsive to treatment with a Wnt pathway inhibitor when the expression level of LEF1 is determined to be above a predetermined level. In some embodiments for the methods described herein, the lung tumor is identified or classified as likely to be responsive or responsive to treatment with a Wnt pathway inhibitor when the expression level of LEF1 is determined to be above a predetermined cut-off point.

In some embodiments, a sample includes, but is not limited to, any clinically relevant tissue sample, such as a tumor biopsy, a core biopsy tissue sample, a fine needle aspirate, a hair follicle, or a sample of bodily fluid, such as blood, plasma, serum, lymph, ascites fluid, cystic fluid, or urine. In some embodiments, the sample is taken from a patient having a tumor or cancer. In some embodiments, the sample is from a primary tumor. In some embodiments, the sample is from a metastasis. The sample may be taken from a human, or from non-human mammals such as, mice, rats, rabbits, non-human primates, canines, felines, ruminants, swine, or sheep. In some embodiments, samples are taken from a subject at multiple time points, for example, before treatment, during treatment, and/or after treatment. In some embodiments, samples are taken from different locations in the subject, for example, a sample from a primary tumor and a sample from a metastasis in a distant location.

In some embodiments, the sample is a paraffin-embedded fixed tissue sample. In some embodiments, the sample is a formalin-fixed paraffin embedded (FFPE) tissue sample. In some embodiments, the sample is a fresh tissue (e.g., tumor) sample. In some embodiments, the sample is a frozen tissue sample. In some embodiments, the sample is a fresh frozen (FF) tissue (e.g., tumor) sample. In some embodiments, the sample is a cell isolated from a fluid. In some embodiments, the sample comprises circulating tumor cells (CTCs). In some embodiments, the sample is an archival tissue sample. In some embodiments, the sample is an archival tissue sample with known diagnosis, treatment, and/or outcome history. In some embodiments, the sample is a block of tissue. In some embodiments, the sample is dispersed cells. In some embodiments, the sample size is from about 1 cell to about 1×10⁶ cells or more. In some embodiments, the sample size is about 10 cells to about 1×10⁵ cells. In some embodiments, the sample size is about 10 cells to about 10,000 cells. In some embodiments, the sample size is about 10 cells to about 1,000 cells. In some embodiments, the sample size is about 10 cells to about 100 cells. In some embodiments, the sample size is about 1 cell to about 10 cells. In some embodiments, the sample size is a single cell.

In some embodiments, the sample is processed to DNA or RNA. In some embodiments. RNA is isolated from the sample. In some embodiments, mRNA is isolated from the sample. In some embodiments, RNA is isolated from cells by procedures that involve cell lysis and denaturation of the proteins contained therein. In some embodiments, DNase is added to remove DNA. In some embodiments, RNase inhibitors are added to the lysis buffer. In some embodiments, a protein denaturation/digestion step is added to the protocol. Methods for preparing total and mRNA are well known in the art and RNA isolation kits are commercially available (e.g., RNeasy mini kit, Qiagen). In some embodiments, the RNA is amplified by PCR-based techniques.

In some embodiments, the sample is processed to a protein lysate. In some embodiments, nuclear proteins are isolated from the sample. In some embodiments, cytoplasmic proteins are isolated from the sample. In some embodiments, one or more proteases are added to the lysate to inhibit denaturation of the proteins. Methods for preparing total and fractionated proteins are well known in the art and reagents and kits are commercially available.

Determination of biomarker expression levels may be performed by any suitable method including, but are not limited to, methods based on analyses of polynucleotide expression, sequencing of polynucleotides, and/or analyses of protein expression. For example, determination of biomarker expression levels may be performed by detecting the expression of mRNA expressed from the genes of interest, and/or by detecting the expression of a polypeptide encoded by the genes.

Commonly used methods for the analysis of polynucleotides, include Southern blot analysis, Northern blot analysis, in situ hybridization, RNase protection assays, polymerase chain reaction (PCR)-based methods, microarray analyses, and sequence-based analyses. Representative methods for sequencing-based gene expression analyses include serial analysis of gene expression (SAGE), massively parallel signature sequencing (MPSS), and NexGen sequencing (NGS), including mRNA sequencing. PCR-based analyses, include but are not limited to, reverse transcription polymerase chain reaction (RT-PCR), quantitative PCR (qPCR) as known as real-time PCR, TaqMan™, TaqMan™ low density array (TLDA), anchored PCR, competitive PCR, rapid amplification of cDNA ends (RACE), differential display, amplified fragment length polymorphism, BeadArray™ technology, high coverage expression profiling (HiCEP) and digital PCR. RT-PCR is a quantitative method that can be used to compare mRNA levels in different samples to examine gene expression profiles. A variation of RT-PCR is real time quantitative PCR, which measures PCR product accumulation through a dual-labeled fluorigenic probe (e.g., TaqMan™ probe).

In certain embodiments, the biomarker expression is determined using RNA sequencing. For example, total RNA is extracted from a fresh sample, from a fresh frozen (FF) tissue sample, or from a macro-dissected formalin-fixed paraffin embedded (FFPE) tissue sample. The quantity and quality of the total RNA is assessed by standard spectrophotometry and/or any other appropriate method (e.g., an Agilent Bioanalyzer). Total RNA is converted into a library of template molecules that include sequencing adapters. The library is hybridized to a flow cell which contains a lawn of covalently bound oligonucleotides complementary to the sequencing adapters. PCR amplification produces discrete clones that can be optically resolved during sequencing. Sequencing by synthesis (SBS) proceeds through multiple cycles of nucleotide incorporation and detection. After sequencing is completed, the reads are aligned to a reference genome. The aligned reads may be further normalized. The data is then analyzed using algorithms and software known to those of skill in the art.

In certain embodiments, the biomarker expression is determined using a qPCR assay. For example, total RNA is extracted from a fresh sample, from a fresh frozen (FF) tissue sample, or from a macro-dissected formalin-fixed paraffin embedded (FFPE) tissue sample. The quantity and quality of the total RNA is assessed by standard spectrophotometry and/or any other appropriate method (e.g., an Agilent Bioanalyzer). Following RNA extraction, the RNA sample is reverse transcribed using standard methods and/or a commercially available eDNA synthesis kit (e.g., Roche Transcriptor First Strand cDNA synthesis kit). The resultant eDNA is pre-amplified using, for example, an ABI pre-amplification kit. Expression of the biomarkers (e.g., LEF1) are assessed on, for example, a Roche Lightcycler 480 system (Roche Diagnostics) using an ABI TaqMan Gene Expression Mastermix. qPCR reactions are performed in triplicate. For each assay a subset of the samples is run without reverse transcription (the RT-neg control), as well as, control samples run without template. A universal human reference RNA sample is included on each plate to act as a positive control. Suitable reference genes are identified from a standard panel of reference genes. Candidate reference genes are selected with different cellular functions to eliminate risk of co-regulation. The most suitable reference genes are evaluated and selected using specific software and algorithms (e.g., Genex software; GeNorm and Normfinder algorithms). The expression level of each biomarker is normalized using the selected optimum reference genes. In some embodiments, these normalized (or standardized) expression values for each biomarker are used to calculate the decision value of the sample. In some embodiments, these normalized (or standardized) expression values for each biomarker are used to calculate an expression level.

In some embodiments, biomarker expression is determined using a PCR-based assay comprising specific primers and/or probes for each biomarker (e.g., LEF1). As used herein, the term “probe” refers to any molecule that is capable of selectively binding a specifically intended target biomolecule. Probes can be synthesized by one of skill in the art using known techniques, or derived from biological preparations. Probes may include but are not limited to, RNA, DNA, proteins, peptides, aptamers, antibodies, and organic molecules. The term “primer” or “probe” encompasses oligonucleotides that have a specific sequence or oligonucleotides that have a sequence complementary to a specific sequence. In some embodiments, the probe is modified. In some embodiments, the probe is modified with a quencher. In some embodiments, the probe is labeled. Labels can include, but are not limited to, colorimetric, fluorescent, chemiluminescent, or bioluminescent labels.

In some embodiments, biomarker expression of each biomarker is determined using a specific primer set and probe. In some embodiments, a specific primer set consists of a forward primer and a reverse primer.

In some embodiments, biomarker expression is measured or determined by a PCR-based assay. In some embodiments, an assay uses one or more primer pairs and probes specific for amplification of LEF1 mRNA. In some embodiments, the primer pairs are a forward (sense) primer and a reverse (anti-sense) primer that consist essentially of at least eight contiguous nucleotides of SEQ ID NO: 14. In some embodiments, a forward and reverse primer pair hybridizes to a nucleotide sequence of SEQ ID NO: 14.

In some embodiments, the expression level of each biomarker (e.g., LEF1) is determined in a separate assay. In some embodiments, the reference gene(s) and normalization methods for each assay are the same for all assays. In some embodiments, the expression levels of several biomarkers are detected in a single multiplex assay.

Alternatively, biomarker expression levels may be determined by amplifying complementary DNA (cDNA) or complementary RNA (cRNA) produced from mRNA and analyzing it using a microarray. Microarray technology allows for simultaneous analysis of the expression of thousands of genes. A number of different array configurations and methods for their production are known to those skilled in the art. In addition, microarrays are commercially available (e.g., Affymetrix GeneChips) or can be custom-produced. Microarrays currently in wide use include cDNA arrays and oligonucleotide arrays. In general, polynucleotides of interest (e.g., probes or probe sets) are plated, or arrayed, on a microchip substrate. In some embodiments, probes to at least 10, 25, 50, 100, 500, 1000, 5000, 10,000, 20,000, or 25,000 or more genes are immobilized on an array substrate. The substrate may be a porous or nonporous support, such as a glass, plastic or gel surface. The probes can include DNA, RNA, copolymer sequences of DNA and RNA, DNA and/or RNA analogues, or combinations thereof. In some embodiments, a microarray includes a support with an ordered array of binding sites for each individual gene. The microarrays can be addressable arrays or positionally addressable arrays, e.g., each probe of the array is located at a known, predetermined position on the solid support such that the identity of each probe can be determined from its position of the array.

Each probe on the microarray can be between 10-50,000 nucleotides in length. In some embodiments, the probes of the microarray can consist of nucleotide sequences with lengths of less than about 1,000 nucleotides, less than about 750 nucleotides, less than about 500 nucleotides, less than about 250 nucleotides, less than about 100 nucleotides, or less than about 50 nucleotides in length. Generally, an array includes positive control probes and negative control probes.

In certain embodiments, the biomarker expression is determined using a microarray. For example, total RNA is extracted from a fresh frozen (FF) tissue sample or total RNA is extracted from a macro-dissected formalin-fixed paraffin embedded (FFPE) tissue sample. The quantity and quality of the total RNA is assessed by standard spectrophotometry and/or any other appropriate technology (e.g., an Agilent Bioanalyzer). Following RNA extraction, the RNA sample is amplified using standard methods and/or a commercially available amplification system (e.g., NuGEN Ovation RNA Amplification System V2). The amplified cDNA is fragmented, labeled, and hybridized to a microarray (e.g., using NuGEN Encore Biotin Module and Affymetrix GeneChip array) following standard procedures. The array is washed, stained, and scanned in accordance with the instructions for the microarray. The microarray data is pre-processed, the probe-level intensity measurements are background corrected, normalized, and summarized as expression measurements using the Robust Multichip algorithm (RMA). The probe level data is summarized to get the expression level of each biomarker (e.g., LEF1). A combination of quality parameter threshold and data reduction techniques (e.g., principal component analysis) is applied to the data set to establish profile quality and identify potential outlying samples. In some embodiments, these normalized (or standardized) expression values for each biomarker are used to calculate the decision value of the sample.

In some embodiments, biomarker expression is analyzed by studying the protein expression of the gene or genes of interest. Commonly used methods for the analysis of protein expression, include but are not limited to, immunohistochemistry (IHC)-based, antibody-based, and mass spectrometry-based methods. Antibodies, generally monoclonal antibodies, may be used to detect expression of a gene product (e.g., protein). In some embodiments, the antibodies can be detected by direct labeling of the antibodies themselves. In other embodiments, an unlabeled primary antibody is used in conjunction with a labeled secondary antibody. Immunohistochemistry methods and/or kits are well known in the art and are commercially available.

In some embodiments, biomarker expression is determined by an assay known to those of skill in the art, including but not limited to, multi-analyte profile test, enzyme-linked immunosorbent assay (ELISA), radioimmunoassay, Western blot assay, immunofluorescent assay, enzyme immunoassay, immunoprecipitation assay, chemiluminescent assay, immunohistochemical assay, dot blot assay, or slot blot assay. In some embodiments, wherein an antibody is used in the assay the antibody is detectably labeled. The antibody labels may include, but are not limited to, immunofluorescent label, chemiluminescent label, phosphorescent label, enzyme label, radiolabel, avidin/biotin, colloidal gold particles, colored particles, and magnetic particles. In some embodiments, biomarker expression is determined by an IHC assay.

In some embodiments, biomarker expression is determined using an agent that specifically binds the biomarker. Any molecular entity that displays specific binding to a biomarker can be employed to determine the level of that biomarker protein in a sample. Specific binding agents include, but are not limited to, antibodies, antibody mimetics, and polynucleotides (e.g., aptamers). One of skill understands that the degree of specificity required is determined by the particular assay used to detect the biomarker protein. In some embodiments, LEF1 expression is determined using an agent that specifically binds LEF1. In some embodiments, the agent used to detect and/or determine LEF1 expression is an anti-LEF1 antibody.

In some embodiments, wherein an antibody is used in the assay the antibody is detectably labeled. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹, ³⁵S, or ³H.

In some embodiments, biomarker expression is determined using an IHC assay. For example, 4 μm-thick FFPE sections are cut from a tumor sample and mounted on coated glass slides. Tissues are deparaffinized and rehydrated by successively incubating them in xylene, 100% ethanol, 95% ethanol, 70% ethanol, and distilled water for antigen retrieval. Slides are placed into retrieval solution and placed in a decloaker for antigen retrieval. To block endogenous peroxidase activity slides are incubated in 6% hydrogen peroxide for 5 minutes and washed in PBS. To block non-specific background staining slides are incubated in blocker for 30 minutes at room temperature. Slides are incubated with an antibody specific for the biomarker. Specific binding is detected using a kit including diaminobenzidine (DAB). The sections are counterstained with hematoxylin. The slides may be analyzed using an automated instrument or evaluated manually by microscope. The staining intensity of each tumor cell (0: no expression, 1: weak expression, 2: moderate expression, 3: strong expression) is measured and cells of each staining level are counted and a percentage for each type is calculated. The data is combined into a weighted H-score for each tissue section: H-score=[3×(%3+cells)]+[2×(%2+cells)]+[1×(%1+cells)]. Using these parameters, the highest score available is H-score=300. In some embodiments, LEF1 expression is determined using an IHC assay. In some embodiments, LEF1 expression is detected using an antibody. In some embodiments, the antibody used in an IHC assay is an anti-LEF1 antibody.

Other suitable methods for analyzing biomarker expression include proteomics-based methods. Proteomics includes, among other things, study of the global changes of protein expression in a sample. In some embodiments, a proteomic method comprises the following steps: (1) separation of individual proteins in a sample by 2-D electrophoresis (2-D PAGE), (2) identification of individual proteins recovered from the gel (e.g., by mass spectrometry or N-terminal sequencing), and (3) analysis of the data using bioinformatics. In some embodiments, a proteomic method comprises using a tissue microarray (TMA). Tissue arrays may be constructed according to a variety of techniques known to one of skill in the art. In certain embodiments, a manual tissue arrayer is used to remove a “core” from a paraffin block prepared from a tissue sample. The core is then inserted into a separate paraffin block in a designated location on a grid. Cores from as many as about 400 samples can be inserted into a single recipient block. The resulting tissue array may be processed into thin sections for analysis. In some embodiments, a proteomic method comprises an antibody microarray. In some embodiments, a proteomic method comprises using mass spectrometry, including but not limited to, SELDI, MALDI, electro spray, and surface plasmon resonance methods. In some embodiments, a proteomic method comprises bead-based technology, including but not limited to, antibodies on beads in an array format. In some embodiments, the proteomic method comprises a reverse phase protein microarray (RPPM). In some embodiments, the proteomic method comprises multiplexed protein profiling, including but not limited to, the Global Proteome Survey (GPS) method.

In some embodiments, the biomarker signature is identified by differential gene expression between two samples. In some embodiments, the biomarker signature is identified by differential gene expression between two samples which comprise genes differentially expressed in cancer cells as compared to normal cells. In some embodiments, the biomarker signature comprises genes differentially expressed in tumorigenic cancer stem cells as compared to non-tumorigenic cancer cells. In some embodiments, the biomarker signature comprises genes differentially expressed in cells from a tumor which is responsive to a specific treatment as compared to cells from a tumor which is non-responsive to the same treatment.

In some embodiments, the gene expression data are refined, filtered, and/or subdivided into biomarker signatures based on statistical analyses. The statistical methods may include, but are not limited to, cluster analysis, supported vector machines (SVM) analysis, supported vector machines—recursive feature elimination (SVM-RFE) analysis, Platt scaling, neural networks, and other algorithms. In some embodiments, the gene expression data are analyzed using a t-test analysis. In some embodiments, the gene expression data are analyzed using paired-sample empirical Baysian analysis. In some embodiments, a combination of statistical analyses is used. In some embodiments, SVM models are used to obtain decision values based on the training data. In some embodiments, classification probabilities for responders and non-responders are obtained using Platt scaling (Platt, 1999, Advances in Large Margin Classifiers, pp. 61-74, MIT Press). Platt scaling may comprise fitting a logistic distribution using maximum likelihood to decision values obtained, for example, by SVM models. In some embodiments, K nearest neighbor (KNN; Altman, 1992, American Statistician, 46:175-185) is used for classification. In some embodiments, a leave-one-out cross-validation (LOOCV) method is used to select a gene signature. In some embodiments, a leave-one-out cross-validation (LOOCV) method is used to measure the performance of a model using a defined gene signature.

In some embodiments, a biomarker signature is obtained by a series of analytical steps. For example, expression data from a training set of samples are obtained from RNA sequencing. The sequencing data are aligned to an annotated human reference genome. The aligned reads are further normalized using a RPKM algorithm. Two-sample Welch's T-test is used for feature selection and KNN is used for classification. Leave-one-out cross-validation (LOOCV) methods are used to identify and select the best predictive genes and also to measure AUC (area under the ROC curve), ACC (accuracy), positive predictive value (PPV), negative predictive value (NPV), sensitivity, and specificity. In an alternative example, expression data from a training set of samples are obtained from microarray analyses. The data are preprocessed to get an expression matrix with specific genes. Genes with near zero variance are removed, as are genes with expression values below a pre-determined level. The remaining genes are ranked using SVM-RFE analysis. Leave-one-out cross-validation (LOOCV) methods are used to identify and select the best predictive genes and also to measure positive predictive value (PPV), negative predictive value (NPV), sensitivity, and specificity.

In some embodiments, the gene expression data and/or biomarker signatures are refined, filtered, and/or subdivided based on additional statistical models. In some embodiments, the gene expression data and/or biomarker signatures are refined, filtered, and/or subdivided based on survival analysis models. These models may include, but are not limited to, Kaplan-Meier survival models, Cox proportional models, Cox proportional hazard models, chi-square analysis, univariate logistic regression models, multivariate competing risk models, linear discriminate analysis models, parametric regression models and correlation analysis models.

In some embodiments, the gene expression data and/or biomarker signatures are refined, filtered, subdivided and/or tested using gene expression array datasets that have associated clinical outcomes. There are several databases that contain datasets that are available to the public, for example, Gene Expression Omnibus (GEO) and ArrayExpress.

In some embodiments, the gene expression data and/or biomarker signatures are refined using biological function parameters, and/or gene sets. For example, in some embodiments, gene expression profiles, and/or biomarker signatures are refined using Gene Set Enrichment Analysis (GSEA) (Subramanian et al., 2005, PNAS. 102: 15545-15550). In some embodiments, the gene expression profiles are refined based on their ability to predict clinical outcome.

In some embodiments of the methods described herein, the Wnt pathway inhibitor is an anti-FZD antibody as described herein. In some embodiments of the methods described herein, the Wnt pathway inhibitor is an antibody that specifically binds at least one human Frizzled (FZD) protein or fragment thereof. In some embodiments, the anti-FZD antibody specifically binds at least one FZD protein selected from the group consisting of: FZD1, FZD2, FZD5, FZD7, and FZD8. In some embodiments, the anti-FZD antibody specifically binds FZD1, FZD2, FZD5, FZD7, and FZD8. In other embodiments, the anti-FZD antibody comprises: (a) a heavy chain CDR1 comprising GFTFSHYTLS (SEQ ID NO: 1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6). In some embodiments, the anti-FZD antibody comprises a heavy chain variable region comprising the amino acids of SEQ ID NO:7. In some embodiments, the anti-FZD antibody comprises a light chain variable region comprising the amino acids of SEQ ID NO:8. In some embodiments, the anti-FZD antibody comprises a heavy chain variable region comprising the amino acids of SEQ ID NO:7 and a light chain variable region comprising the amino acids of SEQ ID NO:8. In some embodiments, the anti-FZD antibody is antibody OMP-18R5. In some embodiments, the anti-FZD antibody is encoded by the plasmid having ATCC deposit no. PTA-9541. In other embodiments, the anti-FZD antibody competes for specific binding to at least one human FZD protein with an antibody encoded by the plasmid deposited with ATCC having deposit no. PTA-9541.

In some embodiments of the methods described herein, the Wnt pathway inhibitor is a soluble receptor. In some embodiments, the Wnt pathway inhibitor comprises the extracellular domain of a FZD receptor protein. In some embodiments, the Wnt pathway inhibitor comprises a Fri domain of a FZD protein. In some embodiments, the Wnt pathway inhibitor comprises the Fri domain of FZD8. In certain embodiments, the Wnt pathway inhibitor comprises the Fri domain of FZD8 and a human Fc domain. In some embodiments, the Wnt pathway inhibitor is the soluble receptor OMP-54F28.

In some embodiments of the methods described herein, the method comprises treating a patient with a Wnt pathway inhibitor described herein (e.g., an anti-FZD antibody), particularly after the patient has been identified as being responsive to treatment with the Wnt pathway inhibitor. In some embodiments, the treatment comprises administering at least one additional therapeutic agent in combination with the Wnt pathway inhibitor. In some embodiments, the at least one additional therapeutic agent comprises 1, 2, 3, or more additional therapeutic agents.

Useful classes of additional therapeutic agents include, for example, antitubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the additional therapeutic agent is an alkylating agent, an antimetabolite, an antimitotic, a topoisomerase inhibitor, or an angiogenesis inhibitor.

Therapeutic agents that may be administered in combination with a Wnt pathway inhibitor include chemotherapeutic agents. Thus, in some embodiments, the method or treatment involves the administration of a Wnt pathway inhibitor in combination with a chemotherapeutic agent or cocktail of multiple different chemotherapeutic agents. Chemotherapeutic agents useful in the instant invention include, but are not limited to, alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate, purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSKI razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum complexes such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (FARESTON); and anti-androgens such as flutamide, nilutamide, bicalutamide, leuprolide, and goserelin; and pharmaceutically acceptable salts, acids or derivatives of any of the above.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (nab-paclitaxel; ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1.

In some embodiments of the methods described herein, the Wnt pathway inhibitor is administered in combination with standard-of-care-chemotherapy for lung cancer as known to those of skill in the art. In some embodiments, the anti-FZD antibody OMP-18R5 is administered in combination with docetaxel. In some embodiments, the anti-FZD antibody OMP-18R5 is administered in combination with paclitaxel. In some embodiments, the anti-FZD antibody OMP-18R5 is administered in combination with pemetrexed. In some embodiments, the anti-FZD antibody OMP-18R5 is administered in combination with cisplatin. In some embodiments, the anti-FZD antibody OMP-18R5 is administered in combination with carboplatin. In some embodiments, the anti-FZD antibody OMP-18R5 is administered in combination with gemcitabine. These combinations as described do not preclude the administration of at least one additional therapeutic agent.

Treatment with a Wnt pathway inhibitor (e.g, an anti-FZD antibody) can occur prior to, concurrently with, or subsequent to administration of chemotherapeutic agents. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4th Edition, 2008, M. C. Perry, Editor, Lippincott, Williams & Wilkins, Philadelphia, Pa.

In some embodiments of the methods described herein, the Wnt pathway inhibitor is administered in combination with an agent such as a small molecule. For example, treatment can involve the combined administration of a Wnt pathway inhibitor with a small molecule that acts as an inhibitor against additional tumor-associated antigens including, but not limited to, EGFR, ErbB2, HER2, and/or VEGF. In certain embodiments, the additional therapeutic agent is a small molecule that inhibits a cancer stem cell pathway. In some embodiments, the additional therapeutic agent is an inhibitor of the Notch pathway. In some embodiments, the additional therapeutic agent is an inhibitor of the RSPO pathway. In some embodiments, the additional therapeutic agent is an inhibitor of the BMP pathway.

In some embodiments of the methods described herein, the Wnt pathway inhibitor is administered in combination with an immunotherapeutic agent. In some embodiments, the immunotherapeutic agent is an immune checkpoint inhibitor. In some embodiments, the immunotherapeutic agent is a PD-1 antagonist, a PD-L1 antagonist, a PD-L2 antagonist, a CTLA-4 antagonist, a CD80 antagonist, a CD86 antagonist, a TIGIT antagonist, a KIR antagonist, a Tim-3 antagonist, a LAG3 antagonist, a CD96 antagonist, a CD20 antagonist, or an IDO1 antagonist. In some embodiments, the PD-1 antagonist is an antibody that specifically binds PD-1. In some embodiments, the antibody that binds PD-1 is pembrolizumab (KEYTRUDA; MK-3475), pidilizumab (CT-011), or nivolumab (BMS-936558). In some embodiments, the PD-L1 antagonist is an antibody that specifically binds PD-L1. In some embodiments, the antibody that binds PD-L1 is RG7446 (MPDL3280A), MEDI4736, or BMS-936559. In some embodiments, the CTLA-4 antagonist is an antibody that specifically binds CTLA-4. In some embodiments, the antibody that binds CTLA-4 is ipilimumab (YERVOY) or tremelimumab (CP-675,206). In some embodiments, the KIR antagonist is an antibody that specifically binds KIR. In some embodiments, the antibody that binds KIR is lirilumab.

Certain embodiments of the present invention comprise a method of identifying a human lung tumor that is likely to be responsive to or non-responsive to treatment with an antibody that specifically binds at least one human frizzled (FZD) selected from the group consisting of FZD1, FZD2, FZD5, FZD7, and FZD8, the method comprising (a) obtaining a sample of the lung tumor; (b) measuring the expression levels of a biomarker signature in the sample, wherein the biomarker signature comprises LEF1; and (c) identifying the lung tumor as likely to be responsive or non-responsive to treatment based on the expression level of the biomarker signature.

In some embodiments, a method of identifying a patient with lung cancer that is likely to be responsive to treatment with an antibody that specifically binds at least one human FZD selected from the group consisting of FZD1, FZD2, FZD5, FZD7, and FZD8, comprises: (a) obtaining a sample of the patient's lung cancer, (b) measuring the expression level of a biomarker signature in the sample, wherein the biomarker signature comprises LEF1; and (c) identifying the patient who is likely to respond to treatment based on the expression level of the biomarker signature.

In some embodiments, a method of selecting a patient with lung cancer for treatment with an antibody that specifically binds at least one human FZD selected from the group consisting of FZD1, FZD2, FZD5, FZD7, and FZD8, comprises: (a) obtaining a sample of the patient's lung cancer; (b) measuring the expression level of a biomarker signature in the sample, wherein the biomarker signature comprises LEF1; (c) selecting the patient for treatment based on the expression level of the biomarker signature.

In some embodiments, a method of treating lung cancer in a patient, comprises: (a) identifying if the patient is likely to respond to treatment with an antibody that specifically binds at least one human FZD selected from the group consisting of FZD1, FZD2, FZD5, FZD7, and FZD8, wherein the identification comprises: (i) obtaining a sample of the patient's lung cancer; (ii) measuring the expression level of a biomarker signature in the sample, wherein the biomarker signature comprises LEF1 and (iii) identifying the patient who is likely to respond to treatment based on the expression level of the biomarker signature; and (b) administering a therapeutically effective amount of the antibody to the patient who is likely to response to treatment.

In certain embodiments, a method of identifying a human lung tumor that is likely to be responsive to or non-responsive to treatment with anti-FZD antibody OMP-18R5 in combination with chemotherapy comprises (a) obtaining a sample of the lung tumor; (b) measuring the expression level of a biomarker signature in the sample, wherein the biomarker signature comprises LEF1; and (c) identifying the lung tumor as likely to be responsive or non-responsive to treatment based on the expression level of the biomarker signature.

In some embodiments, a method of identifying a patient with lung cancer that is likely to be responsive to treatment with the anti-FZD antibody OMP-18R5 in combination with chemotherapy comprises: (a) obtaining a sample of the patient's lung cancer; (b) measuring the expression level of a biomarker signature in the sample, wherein the biomarker signature comprises LEF1; and (c) identifying the patient who is likely to response to treatment based on the expression level of the biomarker signature.

In some embodiments, a method of selecting a patient with lung cancer for treatment with the anti-FZD antibody OMP-18R5 in combination with chemotherapy, comprises: (a) obtaining a sample of the patient's lung cancer; (b) measuring the expression level of a biomarker signature in the sample, wherein the biomarker signature comprises LEF1; (c) selecting the patient for treatment based on the expression level of the biomarker signature.

In some embodiments, a method of treating lung cancer in a patient, comprises: (a) identifying if the patient is likely to respond to treatment with the anti-FZD antibody OMP-18R5 in combination with chemotherapy, wherein the identification comprises: (i) obtaining a sample of the patient's lung cancer; (ii) measuring the expression level of a biomarker signature in the sample, wherein the biomarker signature comprises LEF1; and (iii) identifying the patient who is likely to respond to treatment based on the expression level of the biomarker signature; and (b) administering a therapeutically effective amount of the antibody and chemotherapy to the patient who is likely to respond to treatment.

### III. Wnt Pathway Inhibitors

The present invention provides methods for identifying lung tumors and/or patients with lung cancer that are likely to be responsive to or sensitive to treatment with a Wnt pathway inhibitor. As used herein “Wnt pathway inhibitor” includes, but is not limited to, Frizzled—(FZD) binding agents and Wnt-binding agents. FZD-binding agents may include antibodies that specifically bind FZD proteins. Wnt-binding agents may include antibodies that specifically bind Wnt proteins as well as soluble FZD receptors that bind Wnt proteins.

In certain embodiments, a Wnt pathway inhibitor is an agent that binds one or more human FZD proteins, an FZD-binding agent. In some embodiments, a FZD-binding agent specifically binds one, two, three, four, five, six, seven, eight, nine, or ten FZD proteins. In some embodiments, a FZD-binding agent binds one or more FZD proteins selected from the group consisting of FZD1, FZD2, FZD3, FZD4, FZD5, FZD6, FZD7, FZD8, FZD9, and FZD10. In some embodiments, the FZD-binding agent binds one or more FZD proteins comprising FZD1, FZD2, FZD5, FZD7, and/or FZD8. In certain embodiments, the FZD-binding agent specifically binds FZD1, FZD2, FZD5, FZD7, and FZD8. Non-limiting examples of FZD-binding agents can be found in U.S. Pat. No. 7,982,013.

In certain embodiments, the FZD-binding agent is a FZD antagonist. In certain embodiments, the FZD-binding agent is a Wnt pathway antagonist. In certain embodiments, the FZD-binding agent inhibits Wnt signaling. In some embodiments, the FZD-binding agent inhibits canonical Wnt signaling.

In some embodiments, the FZD-binding agent is an antibody. In some embodiments, the FZD-binding agent is a polypeptide. In certain embodiments, the FZD-binding agent is an antibody or a polypeptide comprising an antigen-binding site. In certain embodiments, an antigen-binding site of a FZD-binding antibody or polypeptide described herein is capable of binding (or binds) one, two, three, four, five, or more human FZD proteins. In certain embodiments, an antigen-binding site of the FZD-binding antibody or polypeptide is capable of specifically binding one, two, three, four, or five human FZD proteins selected from the group consisting of FZD1, FZD2, FZD3, FZD4, FZD5, FZD6, FZD7, FZD8, FZD9 and FZD10. In some embodiments, when the FZD-binding agent is an antibody that binds more than one FZD protein, it may be referred to as a “pan-FZD antibody”.

In certain embodiments, the FZD-binding agent (e.g., antibody) specifically binds the extracellular domain (ECD) of the one or more human FZD proteins to which it binds. In certain embodiments, the FZD-binding agent specifically binds within the Fri domain (also known as the cysteine-rich domain (CRD)) of the one or more human FZD proteins to which it binds.

In certain embodiments, the FZD-binding agent binds one, two, three, four, five, or more FZD proteins. In some embodiments, the FZD-binding agent specifically binds one, two, three, four, or five FZD proteins selected from the group consisting of FZD1, FZD2, FZD5, FZD7, and FZD8. In some embodiments, the FZD-binding agent specifically binds FZD1, FZD2, FZD5, FZD7, and FZD8.

In some embodiments, the FZD-binding agent binds at least one human FZD protein with a dissociation constant (K_{D}) of about 1 μM or less, about 100 nM or less, about 40 nM or less, about 20 nM or less, about 10 nM or less, about 1 nM or less, or about 0.1 nM or less. In some embodiments, a FZD-binding agent binds at least one FZD protein with a K_{D} of about 10 nM or less. In some embodiments, a FZD-binding agent binds at least one FZD protein with a K_{D} of about 1 nM or less. In some embodiments, a FZD-binding agent binds at least one FZD protein with a K_{D} of about 0.1 nM or less. In certain embodiments, a FZD-binding agent binds each of one or more (e.g., 1, 2, 3, 4, or 5) of FZD1, FZD2, FZD5, FZD7, and FZD8 with a K_{D} of about 40 nM or less. In certain embodiments, the FZD-binding agent binds each of one or more of FZD1, FZD2, FZD5, FZD7, and FZD8 with a K_{D} of about 10 nM or less. In certain embodiments, the FZD-binding agent binds each of FZD1, FZD2, FZD5, FZD7, and FZD8 with a K_{D} of about 10 nM. In some embodiments, the K_{D} of the binding agent (e.g., an antibody) to a FZD protein is the K_{D} determined using a FZD-Fc fusion protein comprising at least a portion of the FZD extracellular domain or FZD-Fri domain immobilized on a Biacore chip.

In certain embodiments, the FZD-binding agent binds one or more (for example, two or more, three or more, or four or more) human FZD proteins with an EC₅₀ of about 1 μM or less, about 100 nM or less, about 40 nM or less, about 20 nM or less, about 10 nM or less, or about 1 nM or less. In certain embodiments, a FZD-binding agent binds to more than one FZD protein with an EC₅₀ of about 40 nM or less, about 20 nM or less, or about 10 nM or less. In certain embodiments, the FZD-binding agent has an EC₅₀ of about 20 nM or less with respect to one or more (e.g., 1, 2, 3, 4, or 5) of the following FZD proteins: FZD1, FZD2, FZD5, FZD7, and FZD8. In certain embodiments, the FZD-binding agent has an EC₅₀ of about 10 nM or less with respect to one or more (e.g., 1, 2, 3, 4, or 5) of the following FZD proteins: FZD1, FZD2, FZD5, FZD7, and FZD8.

In certain embodiments, the Wnt pathway inhibitor is a FZD-binding agent which is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is monovalent, monospecific, or bivalent. In some embodiments, the antibody is a bispecific antibody or a multispecific antibody. In some embodiments, the antibody is conjugated to a cytotoxic moiety. In some embodiments, the antibody is isolated. In some embodiments, the antibody is substantially pure.

The FZD-binding agents (e.g., antibodies) of the present invention can be assayed for specific binding by any method known in the art. The immunoassays which can be used include, but are not limited to, competitive and non-competitive assay systems using techniques such as Biacore analyses, FACS analyses, immunofluorescence, immunocytochemistry, Western blot analyses, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well-known in the art (see, e.g., Ausubel et al., Editors, 1994-present, Current Protocols in Molecular Biology, John Wiley & Sons, Inc., New York, N.Y.).

In certain embodiments, the Wnt pathway inhibitor is an antibody that specifically binds FZD1, FZD2, FZD5, FZD7, and/or FZD8, which comprises a heavy chain CDR1 comprising GFTFSHYTLS (SEQ ID NO: 1), a heavy chain CDR2 comprising VISGDGSYTYYADSVKG (SEQ ID NO:2), and a heavy chain CDR3 comprising NFIKYVFAN (SEQ ID NO:3). In some embodiments, the antibody further comprises a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6). In some embodiments, the Wnt pathway inhibitor is an antibody that specifically binds FZD1, FZD2, FZD5, FZD7, and/or FZD8, which comprises a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6). In certain embodiments, the Wnt pathway inhibitor is an antibody that comprises: (a) a heavy chain CDR1 comprising GFTFSHYTLS (SEQ ID NO: 1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6).

In certain embodiments, the Wnt pathway inhibitor is an anti-FZD antibody which comprises: (a) a heavy chain CDR1 comprising GFTFSHYTLS (SEQ ID NO: 1), or a variant thereof comprising 1, 2, 3, or 4 amino acid substitutions; (b) a heavy chain CDR2 comprising VISGDGSYTYYADSVKG (SEQ ID NO:2), or a variant thereof comprising 1, 2, 3, or 4 amino acid substitutions; (c) a heavy chain CDR3 comprising NFIKYVFAN (SEQ ID NO:3), or a variant thereof comprising 1, 2, 3, or 4 amino acid substitutions; (d) a light chain CDR1 comprising SGDNIGSFYVH (SEQ ID NO:4), or a variant thereof comprising 1, 2, 3, or 4 amino acid substitutions; (e) a light chain CDR2 comprising DKSNRPSG (SEQ ID NO:5), or a variant thereof comprising 1, 2, 3, or 4 amino acid substitutions, and (f) a light chain CDR3 comprising QSYANTLSL (SEQ ID NO:6), or a variant thereof comprising 1, 2, 3, or 4 amino acid substitutions. In certain embodiments, the amino acid substitutions are conservative substitutions.

In certain embodiments, the Wnt pathway inhibitor is an anti-FZD antibody which comprises a heavy chain variable region having at least about 80% sequence identity to SEQ ID NO:7 and/or a light chain variable region having at least 80% sequence identity to SEQ ID NO:8. In certain embodiments, the antibody comprises a heavy chain variable region having at least about 85%, at least about 90%& at least about 95%& at least about 97%& or at least about 99% sequence identity to SEQ ID NO:7. In certain embodiments, the antibody comprises a light chain variable region having at least about 85%, at least about 90%, at least about 95%, at least about 97%, or at least about 99/o sequence identity to SEQ ID NO:8. In certain embodiments, the antibody comprises a heavy chain variable region comprising SEQ ID NO:7 and a light chain variable region comprising SEQ ID NO:8. In certain embodiments, the antibody comprises a heavy chain variable region of SEQ ID NO:7 and a light chain variable region of SEQ ID NO:8.

In certain embodiments, the Wnt pathway inhibitor is an anti-FZD antibody which comprises: (a) a heavy chain having at least 90% sequence identity to SEQ ID NO:9 or SEQ ID NO: 11, and/or (b) a light chain having at least 90% sequence identity to SEQ ID NO: 10 or SEQ ID NO: 12. In some embodiments, the antibody comprises: (a) a heavy chain having at least 95% sequence identity to SEQ ID NO:9 or SEQ ID NO: 11; and/or (b) a light chain having at least 95% sequence identity to SEQ ID NO: 10 or SEQ ID NO: 12. In some embodiments, the antibody comprises a heavy chain comprising SEQ ID NO:11 and a light chain comprising SEQ ID NO: 12.

In certain embodiments, the Wnt pathway inhibitor is a FZD-binding agent (e.g., an antibody) that specifically binds at least one of FZD1, FZD2, FZD5, FZD7, and FZD8, wherein the FZD-binding agent (e.g., an antibody) comprises one, two, three, four, five, and/or six of the CDRs of antibody OMP-18R5. Antibody OMP-18R5 (also known as 18R5 and vantictumab), as well as other FZD-binding agents, has been previously described in U.S. Pat. No. 7,982,013. DNA encoding the heavy chain and light chain of the OMP-18R5 IgG2 antibody was deposited with the ATCC, under the conditions of the Budapest Treaty on Sep. 29, 2008, and assigned ATCC deposit designation number PTA-9541. In some embodiments, a FZD-binding agent comprises one or more of the CDRs of OMP-18R5, two or more of the CDRs of OMP-18R5, three or more of the CDRs of OMP-18R5, four or more of the CDRs of OMP-18R5, five or more of the CDRs of OMP-18R5, or all six of the CDRs of OMP-18R5.

The invention provides polypeptides which are Wnt pathway inhibitors. The polypeptides include, but are not limited to, antibodies that specifically bind human FZD proteins. In some embodiments, a polypeptide binds one or more FZD proteins selected from the group consisting of FZD1, FZD2, FZD3, FZD4, FZD5, FZD6, FZD7, FZD8, FZD9, and FZD10. In some embodiments, a polypeptide binds FZD1, FZD2, FZD5, FZD7, and/or FZD8. In some embodiments, a polypeptide binds FZD1, FZD2, FZD5, FZD7, and FZD8.

In certain embodiments, a polypeptide comprises one, two, three, four, five, and/or six of the CDRs of antibody OMP-18R5. In some embodiments, a polypeptide comprises CDRs with up to four (i.e., 0, 1, 2, 3, or 4) amino acid substitutions per CDR. In certain embodiments, the heavy chain CDR(s) are contained within a heavy chain variable region. In certain embodiments, the light chain CDR(s) are contained within a light chain variable region.

In some embodiments, the Wnt pathway inhibitor is a polypeptide that specifically binds one or more human FZD proteins, wherein the polypeptide comprises an amino acid sequence having at least about 80% sequence identity to SEQ ID NO:7, and/or an amino acid sequence having at least about 80% sequence identity to SEQ ID NO:8. In certain embodiments, the polypeptide comprises an amino acid sequence having at least about 85%/o, at least about 90%, at least about 95%, at least about 97%, or at least about 99% sequence identity to SEQ ID NO:7. In certain embodiments, the polypeptide comprises an amino acid sequence having at least about 85%, at least about 90%, at least about 95%, at least about 97%, or at least about 99% sequence identity to SEQ ID NO:8. In certain embodiments, the polypeptide comprises an amino acid sequence having at least about 95% sequence identity to SEQ ID NO:7 and/or an amino acid sequence having at least about 95% sequence identity to SEQ ID NO:8. In certain embodiments, the polypeptide comprises an amino acid sequence comprising SEQ ID NO:7 and/or an amino acid sequence comprising SEQ ID NO:8.

In some embodiments, a polypeptide comprises a sequence selected from the group consisting of: SEQ ID NO:7, SEQ ID NO:8, SEQ ID NO:9, SEQ ID NO: 10, SEQ ID NO: 11, and SEQ ID NO: 12. In some embodiments, a Wnt pathway inhibitor comprises a polypeptide comprising a sequence selected from the group consisting of: SEQ ID NO:7, SEQ ID NO:8, SEQ ID NO:9, SEQ ID NO: 10, SEQ ID NO: 11, and SEQ ID NO: 12.

In certain embodiments, a polypeptide comprises the heavy chain variable region and light chain variable region of the OMP-18R5 antibody. In certain embodiments, a polypeptide comprises the heavy chain and light chain of the OMP-18R5 antibody (with or without the leader sequence). In certain embodiments, a Wnt pathway inhibitor comprises a polypeptide comprising the heavy chain variable region and light chain variable region of the OMP-18R5 antibody. In certain embodiments, a Wnt pathway inhibitor comprises a polypeptide comprising the heavy chain and light chain of the OMP-18R5 antibody (with or without the leader sequence).

In certain embodiments, a Wnt pathway inhibitor comprises, consists essentially of, or consists of, the antibody OMP-18R5.

In certain embodiments, a Wnt pathway inhibitor competes for specific binding to one or more human FZD proteins with an antibody that comprises a heavy chain variable region comprising SEQ ID NO:7 and a light chain variable region comprising SEQ ID NO:8. In certain embodiments, a Wnt pathway inhibitor competes for specific binding to one or more human FZD proteins with an antibody that comprises a heavy chain comprising SEQ ID NO:9 (with or without the signal sequence) and a light chain comprising SEQ ID NO: 10 (with or without the signal sequence). In certain embodiments, a Wnt pathway inhibitor competes for specific binding to one or more human FZD proteins with an antibody that comprises a heavy chain comprising SEQ ID NO: 11 and a light chain comprising SEQ ID NO: 12. In certain embodiments, a Wnt pathway inhibitor competes with antibody OMP-18R5 for specific binding to one or more human FZD proteins. In some embodiments, a Wnt pathway inhibitor competes for specific binding to one or more human FZD proteins in an in vitro competitive binding assay.

In certain embodiments, a Wnt pathway inhibitor binds the same epitope, or essentially the same epitope, on one or more human FZD proteins as antibody 18R5. In another embodiment, a Wnt pathway inhibitor binds an epitope on one or more human FZD proteins that overlaps with the epitope on a FZD protein bound by antibody 18R5.

In some embodiments, the Wnt pathway inhibitors are polyclonal antibodies. Polyclonal antibodies can be prepared by any known method. In some embodiments, polyclonal antibodies are raised by immunizing an animal (e.g., a rabbit, rat, mouse, goat, donkey) by multiple subcutaneous or intraperitoneal injections of an antigen of interest (e.g., a purified peptide fragment, full-length recombinant protein, or fusion protein). The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood and/or ascites of the immunized animal. The polyclonal antibodies can be purified from serum or ascites according to standard methods in the art including, but not limited to, affinity chromatography, ion-exchange chromatography, gel electrophoresis, and dialysis.

In some embodiments, the Wnt pathway inhibitors are monoclonal antibodies. Monoclonal antibodies can be prepared using hybridoma methods known to one of skill in the art. In some embodiments, using the hybridoma method, a mouse, hamster, or other appropriate host animal, is immunized as described above to elicit the production of antibodies that will specifically bind the immunizing antigen. In some embodiments, isolated lymphocytes can be immunized in vitro. In some embodiments, the immunizing antigen can be a human protein or a fragment thereof. In some embodiments, the immunizing antigen can be a mouse protein or a fragment thereof.

Following immunization, lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen may be identified by a variety of methods including, but not limited to, immunoprecipitation, immunoblotting, and in vitro binding assay (e.g., flow cytometry, FACS, ELISA, and radioimmunoassay). The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be modified to increase affinity, also known as “affinity-matured” or “affinity maturation”. Affinity-matured antibodies may be produced by a variety of procedures known in the art. For example, techniques may include affinity maturation by heavy chain and light chain variable region domain shuffling, random mutagenesis of CDRs, random mutagenesis of framework residues, and/or site-directed mutagenesis.

In certain embodiments, monoclonal antibodies can be made using recombinant DNA techniques known to one skilled in the art. The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins. In other embodiments, recombinant monoclonal antibodies, or fragments thereof, can be isolated from phage display libraries.

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted for those regions of, for example, a human antibody to generate a chimeric antibody, or for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In some embodiments, the Wnt pathway inhibitor is a humanized antibody. Typically, humanized antibodies are human immunoglobulins in which amino acid residues of the CDRs are replaced by amino acid residues of CDRs from an immunoglobulin of a non-human species (e.g., mouse, rat, rabbit, hamster, etc.) that have the desired specificity, affinity, and/or binding capability. In some embodiments, framework variable region amino acid residues of a human immunoglobulin are replaced with corresponding amino acid residues from an antibody of a non-human species. In some embodiments, the humanized antibody can be further modified by the substitution of additional amino acid residues either in the framework variable region and/or within the replaced non-human amino acid residues to further refine and optimize antibody specificity, affinity, and/or binding capability. In general, a humanized antibody will comprise all, or substantially all, of the CDRs that correspond to the non-human immunoglobulin whereas all, or substantially all, of the framework variable regions are those of a human immunoglobulin sequence. In some embodiments, the humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject. Methods used to generate humanized antibodies are well known in the art.

In certain embodiments, the Wnt pathway inhibitor is a human antibody. Human antibodies can be directly prepared using various techniques known in the art. In some embodiments, immortalized human B lymphocytes immunized in vitro or isolated from an immunized individual that produces an antibody directed against a target antigen can be generated. In some embodiments, the human antibody can be selected from a phage library, where that phage library expresses human antibodies. Alternatively, phage display technology can be used to produce human antibodies and antibody fragments in vitro, from immunoglobulin variable domain gene repertoires from unimmunized donors. Techniques for the generation and use of antibody phage libraries are well-known in the art. Affinity maturation strategies including, but not limited to, chain shuffling and site-directed mutagenesis, are known in the art and may be employed to generate high affinity human antibodies.

In some embodiments, human antibodies can be made in transgenic mice that contain human immunoglobulin loci. These mice are capable, upon immunization, of producing the full repertoire of human antibodies in the absence of endogenous immunoglobulin production.

This invention also encompasses bispecific antibodies that specifically recognize at least one human FZD protein. Bispecific antibodies are capable of specifically recognizing and binding at least two different epitopes. The different epitopes can either be within the same molecule (e.g., two different epitopes on human FZD7) or on different molecules (e.g., one epitope on FZD7 and a different epitope on a second protein). In some embodiments, the bispecific antibodies are monoclonal antibodies. In some embodiments, the bispecific antibodies are humanized antibodies. In some embodiments, the bispecific antibodies are human antibodies. In some embodiments, the antibodies can specifically recognize and bind a first antigen target, (e.g., a FZD protein) as well as a second antigen target on the same cell or a different cell. In some embodiments, the antibodies can be used to direct cytotoxic agents to cells which express a particular target antigen. In some embodiments, the antibodies possess an antigen-binding arm and an arm which binds a cytotoxic agent or a radionuclide chelator, such as EOTUBE, DPTA, DOTA, or TETA.

Bispecific antibodies can be intact antibodies or antibody fragments. Antibodies with more than two valencies are also contemplated (e.g., trispecific antibodies). Thus, in certain embodiments the antibodies are multispecific. Techniques for making bispecific and multispecific antibodies are known by those skilled in the art.

In certain embodiments, the antibodies (or other polypeptides) described herein may be monospecific. For example, in certain embodiments, each of the one or more antigen-binding sites that an antibody contains is capable of binding (or binds) a homologous epitope on different proteins. In certain embodiments, an antigen-binding site of a monospecific antibody described herein is capable of binding (or binds), for example, FZD5 and FZD7 (i.e., the same epitope is found on both FZD5 and FZD7 proteins).

In certain embodiments, the Wnt pathway inhibitor is an antibody fragment comprising an antigen-binding site. Antibody fragments may have different functions or capabilities than intact antibodies, for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries to allow rapid and effective identification of monoclonal Fab fragments with the desired specificity for a FZD protein or derivatives, fragments, analogs or homologs thereof. In some embodiments, antibody fragments are linear antibody fragments. In certain embodiments, antibody fragments are monospecific or bispecific. In certain embodiments, the Wnt pathway inhibitor is a scFv. Various techniques can be used for the production of single-chain antibodies specific to one or more human FZD proteins.

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis). In some embodiments, an antibody is modified to decrease its serum half-life.

Heteroconjugate antibodies are also within the scope of the present invention. Heteroconjugate antibodies are composed of two covalently joined antibodies. Such antibodies have, for example, been proposed to target immune cells to tumor cells. It is also contemplated that the heteroconjugate antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified antibodies can comprise any type of variable region that provides for the association of the antibody with the target (i.e., a human FZD protein). In this regard, the variable region may comprise or be derived from any type of mammal that can be induced to mount a humoral response and generate immunoglobulins against the desired tumor-associated antigen. As such, the variable region of the modified antibodies can be, for example, of human, murine, non-human primate (e.g. cynomolgus monkeys, macaques, etc.), or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid residues or sequences.

In certain embodiments, the variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs will be derived preferably from an antibody from a different species. It may not be necessary to replace all of the CDRs with all of the CDRs from the donor variable region to transfer the antigen binding capacity of one variable domain to another. Rather, it may only be necessary to transfer those residues that are necessary to maintain the activity of the antigen-binding site.

Alterations to the variable region notwithstanding, those skilled in the art will appreciate that the modified antibodies of this invention will comprise antibodies (e.g., full-length antibodies or immunoreactive fragments thereof) in which at least a fraction of one or more of the constant region domains has been deleted or otherwise altered so as to provide desired biochemical characteristics such as increased tumor localization and/or increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. The modified antibodies disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In some embodiments, the modified antibodies will comprise domain deleted constructs or variants wherein the entire CH2 domain has been removed (ΔCH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete the part of one or more constant region domains that control a specific effector function (e.g. complement C1q binding). Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells, release of inflammatory mediators, placental transfer, and control of immunoglobulin production.

In certain embodiments, the Wnt pathway inhibitors are antibodies that provide for altered effector functions. These altered effector functions may affect the biological profile of the administered antibody. For example, in some embodiments, the deletion or inactivation (through point mutations or other means) of a constant region domain may reduce Fc receptor binding of the circulating modified antibody (e.g., anti-FZD antibody) thereby increasing cancer cell localization and/or tumor penetration. In other embodiments, the constant region modifications increase or reduce the serum half-life of the antibody. In some embodiments, the constant region is modified to eliminate disulfide linkages or oligosaccharide moieties. Modifications to the constant region in accordance with this invention may easily be made using well known biochemical or molecular engineering techniques well within the purview of the skilled artisan.

In certain embodiments, a Wnt pathway inhibitor is an antibody does not have one or more effector functions. For instance, in some embodiments, the antibody has no ADCC activity and/or no CDC activity. In certain embodiments, the antibody does not bind an Fc receptor and/or complement factors. In certain embodiments, the antibody has no effector function.

The present invention further embraces variants and equivalents which are substantially homologous to the chimeric, humanized, and human antibodies, or antibody fragments thereof, set forth herein. These can contain, for example, conservative substitution mutations.

In certain embodiments, the antibodies described herein are isolated. In certain embodiments, the antibodies described herein are substantially pure.

In certain embodiments, the Wnt pathway inhibitor is a soluble receptor. In certain embodiments, the Wnt pathway inhibitor comprises the extracellular domain of a human FZD receptor protein. In some embodiments, the Wnt pathway inhibitor comprises a Fri domain of a human FZD protein. In certain embodiments, the human FZD protein is FZD1, FZD2, FZD3, FZD4, FZD5, FZD6, FZD7, FZD8, FZD9, or FZD10. In some embodiments, the human FZD protein is FZD8. Non-limiting examples of soluble FZD receptors can be found in U.S. Pat. Nos. 7,723,477 and 7,947,277 and U.S. Patent Publication No. 2013/0034551.

The predicted Fri domain of human FZD8 is provided as SEQ ID NO: 16. Those of skill in the art may differ in their understanding of the exact amino acids corresponding to the various Fri domains. Thus, the N-terminus and/or C-terminus of the FZD8 Fri domain defined by SEQ ID NO: 16 may extend or be shortened by 1, 2, 3, 4, 5, 6, 7, 8, 9, or even 10 amino acids.

In certain embodiments, the Wnt pathway inhibitor comprises the Fri domain of human FZD8. In some embodiments, the Wnt pathway inhibitor comprises a Fri domain consisting essentially of the Fri domain of FZD8. In some embodiments, the Wnt pathway inhibitor comprises SEQ ID NO: 16. In some embodiments, the Wnt pathway inhibitor comprises a Fri domain consisting essentially of SEQ ID NO: 16.

In certain embodiments, the Wnt pathway inhibitor comprises a variant of the aforementioned FZD8 Fri domain sequences that comprises one or more (e.g., one, two, three, four, five, six, seven, eight, nine, ten, etc.) conservative substitutions and is capable of binding Wnt protein(s).

In certain embodiments, a Wnt pathway inhibitor, such as an agent comprising a Fri domain of a human FZD receptor, further comprises a non-FZD polypeptide (i.e., a heterologous polypeptide). In some embodiments, a FZD soluble receptor may include a FZD ECD or a Fri domain linked to another non-FZD functional and structural polypeptide including, but not limited to, a human Fc region, a protein tag (e.g., myc, FLAG, GST, GFP), other endogenous proteins or protein fragments, or any other useful protein sequence including any linker region between a FZD ECD or Fri domain and a second polypeptide. In certain embodiments, the non-FZD polypeptide comprises a human Fc region. The Fc region can be obtained from any of the classes of immunoglobulin, IgG, IgA, IgM, IgD, and IgE. In some embodiments, the Fc region is a human IgG1 Fc region. In some embodiments, the Fe region is a human IgG2 Fc region. In some embodiments, the Fc region is a wild-type Fc region. In some embodiments, the Fc region is a mutated Fc region. In some embodiments, the Fc region is truncated at the N-terminal end by 1, 2, 3, 4, 5, 6, 7, 8, 9, or 10 amino acids, (e.g., in the hinge domain). In some embodiments, an amino acid in the hinge domain is changed to hinder undesirable disulfide bond formation. In some embodiments, a cysteine is replaced with a serine to hinder or block undesirable disulfide bond formation. In some embodiments, the Fc region is truncated at the C-terminal end by 1, 2, 3, or more amino acids. In some embodiments, the Fc region is truncated at the C-terminal end by 1 amino acid. In certain embodiments, the non-FZD polypeptide comprises SEQ ID NO: 17, SEQ ID NO: 18, SEQ ID NO: 19, SEQ ID NO:20, or SEQ ID NO:21. In certain embodiments, the non-FZD polypeptide consists essentially of SEQ ID NO: 17, SEQ ID NO: 18, SEQ ID NO: 19, SEQ ID NO:20, or SEQ ID NO:21.

In certain embodiments, a Wnt pathway inhibitor is a fusion protein comprising at least a minimal Fri domain of a FZD receptor and a Fc region. As used herein, a “fusion protein” is a hybrid protein expressed by a nucleic acid molecule comprising nucleotide sequences of at least two genes. In some embodiments, the C-terminus of the first polypeptide is linked to the N-terminus of the immunoglobulin Fc region. In some embodiments, the first polypeptide (e.g., a FZD Fri domain) is directly linked to the Fc region (i.e. without an intervening linker). In some embodiments, the first polypeptide is linked to the Fc region via a linker.

As used herein, the term “linker” refers to a linker inserted between a first polypeptide (e.g., a FZD component) and a second polypeptide (e.g., a Fc region). In some embodiments, the linker is a peptide linker. Linkers should not adversely affect the expression, secretion, or bioactivity of the polypeptide. Linkers should not be antigenic and should not elicit an immune response. Suitable linkers are known to those of skill in the art and often include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. As used herein, a “linker” is an intervening peptide sequence that does not include amino acid residues from either the C-terminus of the first polypeptide (e.g., a FZD Fri domain) or the N-terminus of the second polypeptide (e.g., the Fc region).

FZD proteins contain a signal sequence that directs the transport of the proteins. Signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site may be recognized and/or used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides described herein may comprise a mixture of polypeptides with different N-termini. In some embodiments, the N-termini differ in length by 1, 2, 3, 4, 5, 6, 7, 8, 9, 10, or more amino acids. In some embodiments, the N-termini differ in length by 1, 2, 3, 4, or 5 amino acids. In some embodiments, the polypeptide is substantially homogeneous, i.e., the polypeptides have the same N-terminus. In some embodiments, the signal sequence of the polypeptide comprises one or more (e.g., one, two, three, four, five, six, seven, eight, nine, ten, etc.) amino acid substitutions and/or deletions. In some embodiments, the signal sequence of the polypeptide comprises amino acid substitutions and/or deletions that allow one cleavage site to be dominant, thereby resulting in a substantially homogeneous polypeptide with one N-terminus.

In some embodiments, the Wnt pathway inhibitor comprises SEQ ID NO: 16 and SEQ ID NO:17, SEQ ID NO:18, SEQ ID NO:19. SEQ ID NO:20, or SEQ ID NO:21. In some embodiments, the Wnt pathway inhibitor comprising SEQ ID NO: 16 and SEQ ID NO: 17. In some embodiments, the Wnt pathway inhibitor comprising SEQ ID NO: 16 and SEQ ID NO: 18. In some embodiments, the Wnt pathway inhibitor comprising SEQ ID NO: 16 and SEQ ID NO: 19. In some embodiments, the Wnt pathway inhibitor comprising SEQ ID NO: 16 and SEQ ID NO:20. In some embodiments, the Wnt pathway inhibitor comprising SEQ ID NO: 16 and SEQ ID NO:21. In some embodiments, the Wnt pathway inhibitor comprises an amino acid sequence selected from the group consisting of: SEQ ID NO:22 or SEQ ID NO:23. In some embodiments, the Wnt pathway inhibitor comprises SEQ ID NO:23.

In certain embodiments, the Wnt pathway inhibitor comprises the sequence of SEQ ID NO:22. In certain embodiments, the agent comprises the sequence of SEQ ID NO:22, comprising one or more (e.g., one, two, three, four, five, six, seven, eight, nine, ten, etc.) conservative substitutions. In certain embodiments, the Wnt pathway inhibitor comprises a sequence having at least about 90%, about 95%, or about 98% sequence identity with SEQ ID NO:22. In certain embodiments, the variants of SEQ ID NO:22 maintain the ability to bind one or more human Wnt proteins.

In some embodiments, the Wnt pathway inhibitor is OMP-54F28.

In certain embodiments, a Wnt pathway inhibitor is a polypeptide comprising an amino acid sequence selected from the group consisting of: SEQ ID NO:22 and SEQ ID NO:23. In certain embodiments, the polypeptide comprises an amino acid sequence of SEQ ID NO:22. In some embodiments, a polypeptide consists essentially of an amino acid sequence of SEQ ID NO:22.

In some embodiments, the polypeptide is a substantially purified polypeptide comprising an amino acid sequence of SEQ ID NO:22. In certain embodiments, the substantially purified polypeptide consists of at least 90% of a polypeptide that has an N-terminal amino acid sequence of ASA (alanine-serine-alanine). In some embodiments, the nascent polypeptide comprises a signal sequence that results in a substantially homogeneous polypeptide product with one N-terminal sequence.

In certain embodiments, a Wnt pathway inhibitor comprises a Fc region of an immunoglobulin. Those skilled in the art will appreciate that some of the binding agents of this invention will comprise fusion proteins in which at least a portion of the Fc region has been deleted or otherwise altered so as to provide desired biochemical characteristics, such as increased cancer cell localization, increased tumor penetration, reduced serum half-life, or increased serum half-life, when compared with a fusion protein of approximately the same immunogenicity comprising a native or unaltered constant region. Modifications to the Fc region may include additions, deletions, or substitutions of one or more amino acids in one or more domains. The modified fusion proteins disclosed herein may comprise alterations or modifications to one or more of the two heavy chain constant domains (CH2 or CH3) or to the hinge region. In other embodiments, the entire CH2 domain may be removed (ΔCH2 constructs). In some embodiments, the omitted constant region domain is replaced by a short amino acid spacer (e.g., 10 aa residues) that provides some of the molecular flexibility typically imparted by the absent constant region domain.

In some embodiments, the modified fusion proteins are engineered to link the CH3 domain directly to the hinge region. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the fusion protein.

In some embodiments, the modified fusion proteins may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete that part of one or more constant region domains that control a specific effector function (e.g., complement C1q binding). Such partial deletions of the constant regions may improve selected characteristics of the binding agent (e.g., serum half-life) while leaving other desirable functions associated with the subject constant region domain intact. Moreover, as alluded to above, the constant regions of the disclosed fusion proteins may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified fusion protein. In certain embodiments, the modified fusion proteins comprise the addition of one or more amino acids to the constant region to enhance desirable characteristics such as decreasing or increasing effector function, or provide for more cytotoxin or carbohydrate attachment sites.

In some embodiments, a modified fusion protein provides for altered effector functions that, in turn, affect the biological profile of the administered agent. For example, in some embodiments, the deletion or inactivation (through point mutations or other means) of a constant region domain may reduce Fc receptor binding of the circulating modified agent, thereby increasing cancer cell localization and/or tumor penetration. In other embodiments, the constant region modifications increase or reduce the serum half-life of the agent. In some embodiments, the constant region is modified to eliminate disulfide linkages or oligosaccharide moieties.

In certain embodiments, a modified fusion protein does not have one or more effector functions normally associated with an Fc region. In some embodiments, the agent has no antibody-dependent cell-mediated cytotoxicity (ADCC) activity, and/or no complement-dependent cytotoxicity (CDC) activity. In certain embodiments, the agent does not bind to the Fc receptor and/or complement factors. In certain embodiments, the agent has no effector function.

In some embodiments, the Wnt pathway inhibitor (e.g., a soluble receptor) is modified to reduce immunogenicity. In general, immune responses against completely normal human proteins are rare when these proteins are used as therapeutics. However, although many fusion proteins comprise polypeptides sequences that are the same as the sequences found in nature, several therapeutic fusion proteins have been shown to be immunogenic in mammals. In some studies, a fusion protein comprising a linker has been found to be more immunogenic than a fusion protein that does not contain a linker. Accordingly, in some embodiments, the polypeptides of the invention are analyzed by computation methods to predict immunogenicity. In some embodiments, the polypeptides are analyzed for the presence of T-cell and/or B-cell epitopes. If any T-cell or B-cell epitopes are identified and/or predicted, modifications to these regions (e.g., amino acid substitutions) may be made to disrupt or destroy the epitopes. Various algorithms and software that can be used to predict T-cell and/or B-cell epitopes are known in the art. For example, the software programs SYFPEITHI, HLA Bind, PEPVAC, RANKPEP, DiscoTope, ElliPro, and Antibody Epitope Prediction are all publicly available.

The isolated polypeptides that can be used in the methods described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof.

In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding binding agents (e.g., antibodies or soluble receptors), or fragments thereof, against one or more human FZD proteins. For example, recombinant expression vectors can be replicable DNA constructs which have synthetic or cDNA-derived DNA fragments encoding a polypeptide chain of a FZD-binding agent or an anti-FZD antibody or fragment thereof, operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence, or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of a FZD-binding agent (or a protein to use as an antigen) include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts are well-known in the art.

Various mammalian culture systems are used to express recombinant polypeptides. Expression of recombinant proteins in mammalian cells may be preferred because such proteins are generally correctly folded, appropriately modified, and biologically functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Thus, the present invention provides cells comprising the FZD-binding agents described herein. In some embodiments, the cells produce the binding agents (e.g., antibodies) described herein. In certain embodiments, the cells produce an antibody. In certain embodiments, the cells produce antibody OMP-18R5 (vantictumab).

The proteins produced by a host cell can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Isolated proteins can also be physically characterized using such techniques as proteolysis, mass spectrometry (MS), nuclear magnetic resonance (NMR), high performance liquid chromatography (HPLC), and x-ray crystallography.

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify a binding agent. Some or all of the foregoing purification steps, in various combinations, can also be employed to provide a homogeneous recombinant protein.

In some embodiments, recombinant protein produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

In certain embodiments, the FZD-binding agent is a polypeptide that is not an antibody. A variety of methods for identifying and producing non-antibody polypeptides that bind with high affinity to a protein target are known in the art. In certain embodiments, phage display technology may be used to produce and/or identify a FZD-binding polypeptide. In certain embodiments, the polypeptide comprises a protein scaffold of a type selected from the group consisting of protein A, protein G, a lipocalin, a fibronectin domain, an ankyrin consensus repeat domain, and thioredoxin.

In certain embodiments, the binding agents can be used in any one of a number of conjugated (i.e. an immunoconjugate or radioconjugate) or non-conjugated forms. In certain embodiments, antibodies can be used in a non-conjugated form to harness the subject's natural defense mechanisms including complement-dependent cytotoxicity and antibody dependent cellular toxicity to eliminate the malignant or cancer cells.

In some embodiments, the binding agent is conjugated to a cytotoxic agent. In some embodiments, the cytotoxic agent is a chemotherapeutic agent including, but not limited to, methotrexate, adriamycin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments, the cytotoxic agent is a radioisotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re and ²¹²Bi.

In some embodiments, conjugates of an antibody and one or more small molecule toxins, such as a calicheamicin, maytansinoids, a trichothene, and CC1065, and the derivatives of these toxins that have toxin activity, can be produced.

In certain embodiments, conjugates of an antibody and a cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In certain embodiments, the Wnt pathway inhibitor (e.g., an anti-FZD antibody) is an antagonist of at least one Wnt protein (i.e., 1, 2, 3, 4, 5, 6, 7, 8, 9, or 10 Wnt proteins). In certain embodiments, the Wnt pathway inhibitor inhibits activity of the Wnt protein(s) to which it binds. In certain embodiments, the Wnt pathway inhibitor inhibits at least about 10%, at least about 20%, at least about 30%, at least about 50%, at least about 75%, at least about 90% or about 100% of the activity of the human Wnt protein(s) to which it binds.

In certain embodiments, the Wnt pathway inhibitor (e.g., an anti-FZD antibody) inhibits binding of at least one human Wnt to an appropriate receptor. In certain embodiments, the Wnt pathway inhibitor inhibits binding of at least one human Wnt protein to one or more human FZD proteins. In some embodiments, the at least one Wnt protein is selected from the group consisting of: Wnt1, Wnt2, Wnt2b/13, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In some embodiments, the one or more human FZD proteins are selected from the group consisting of: FZD1, FZD2, FZD3, FZD4, FZD5, FZD6, FZD7, FZD8, FZD9, and FZD10. In certain embodiments, the Wnt pathway inhibitor inhibits binding of one or more Wnt proteins to FZD1, FZD2, FZD5, FZD7, and/or FZD8. In certain embodiments, the inhibition of binding of a particular Wnt to a FZD protein by a Wnt pathway inhibitor is at least about 10%, at least about 25%, at least about 50%, at least about 75%, at least about 90%, or at least about 95%. In certain embodiments, an agent that inhibits binding of a Wnt to a FZD protein also inhibits Wnt pathway signaling. In certain embodiments, a Wnt pathway inhibitor that inhibits human Wnt pathway signaling is an antibody.

In certain embodiments, the Wnt pathway inhibitor is an antagonist of at least one human FZD protein and inhibits FZD activity. In certain embodiments, the Wnt pathway inhibitor inhibits FZD activity by at least about 10%, at least about 20%, at least about 30%, at least about 50%, at least about 75%, at least about 90%, or about 100%. In some embodiments, the Wnt pathway inhibitor inhibits activity of one, two, three, four, five or more FZD proteins. In some embodiments, the Wnt pathway inhibitor inhibits activity of at least one human FZD protein selected from the group consisting of: FZD1, FZD2, FZD3, FZD4, FZD5, FZD6, FZD7, FZD8, FZD9, and FZD10. In certain embodiments, the Wnt pathway inhibitor inhibits activity of FZD1, FZD2, FZD5, FZD7, and/or FZD8. In some embodiments, the Wnt pathway inhibitor is an anti-FZD antibody. In certain embodiments, the Wnt pathway inhibitor is anti-FZD antibody OMP-18R5.

In certain embodiments, a Wnt pathway inhibitor is an antagonist of β-catenin signaling. In certain embodiments, the Wnt pathway inhibitor inhibits β-catenin signaling by at least about 10%, at least about 20%, at least about 30%, at least about 50%, at least about 75%& at least about 90%, or about 100%. In certain embodiments, a Wnt pathway inhibitor that inhibits β-catenin signaling is an antibody. In certain embodiments, a Wnt pathway inhibitor that inhibits β-catenin signaling is an anti-FZD antibody. In certain embodiments, a Wnt pathway inhibitor that inhibits β-catenin signaling is antibody OMP-18R5.

In certain embodiments, the Wnt pathway inhibitor inhibits binding of at least one Wnt protein to a receptor. In certain embodiments, the Wnt pathway inhibitor inhibits binding of at least one human Wnt protein to one or more of its receptors. In some embodiments, the Wnt pathway inhibitor inhibits binding of at least one Wnt protein to at least one FZD protein. In some embodiments, the Wnt-binding agent inhibits binding of at least one Wnt protein to FZD1, FZD2, FZD3, FZD4, FDZ5, FDZ6, FDZ7, FDZ8, FDZ9, and/or FZD10. In certain embodiments, the inhibition of binding of at least one Wnt to at least one FZD protein is at least about 10%, at least about 25%, at least about 50%, at least about 75%, at least about 90%, or at least about 95%. In certain embodiments, a Wnt pathway inhibitor that inhibits binding of at least one Wnt to at least one FZD protein further inhibits Wnt pathway signaling and/or β-catenin signaling. In certain embodiments, a Wnt pathway inhibitor that inhibits binding of at least one human Wnt to at least one FZD protein is an antibody. In certain embodiments, a Wnt pathway inhibitor that inhibits binding of at least one human Wnt to at least one FZD protein is an anti-FZD antibody. In certain embodiments, a Wnt pathway inhibitor that inhibits binding of at least one human Wnt to at least one FZD protein is antibody OMP-18R5.

In certain embodiments, the Wnt pathway inhibitor blocks binding of at least one Wnt to a receptor. In certain embodiments, the Wnt pathway inhibitor blocks binding of at least one human Wnt protein to one or more of its receptors. In some embodiments, the Wnt pathway inhibitor blocks binding of at least one Wnt to at least one FZD protein. In some embodiments, the Wnt pathway inhibitor blocks binding of at least one Wnt protein to FZD1, FZD2, FZD3, FZD4, FDZ5, FDZ6, FDZ7, FDZ8, FDZ9, and/or FZD10. In certain embodiments, the blocking of binding of at least one Wnt to at least one FZD protein is at least about 10%, at least about 25%, at least about 50%, at least about 75%, at least about 90%, or at least about 95%. In certain embodiments, a Wnt pathway inhibitor that blocks binding of at least one Wnt protein to at least one FZD protein further inhibits Wnt pathway signaling and/or β-catenin signaling. In certain embodiments, a Wnt pathway inhibitor that blocks binding of at least one human Wnt to at least one FZD protein is an antibody. In certain embodiments, a Wnt pathway inhibitor that blocks binding of at least one human Wnt to at least one FZD protein is an anti-FZD antibody. In certain embodiments, a Wnt pathway inhibitor that blocks binding of at least one human Wnt to at least one FZD protein is antibody OMP-18R5.

In certain embodiments, the Wnt pathway inhibitor inhibits Wnt pathway signaling. It is understood that a Wnt pathway inhibitor that inhibits Wnt pathway signaling may, in certain embodiments, inhibit signaling by one or more receptors in the Wnt signaling pathway but not necessarily inhibit signaling by all receptors. In certain alternative embodiments, Wnt pathway signaling by all human receptors may be inhibited. In certain embodiments, Wnt pathway signaling by one or more receptors selected from the group consisting of FZD1, FZD2, FZD3, FZD4, FDZ5, FDZ6, FDZ7, FDZ8, FDZ9, and FZD10 is inhibited. In certain embodiments, the inhibition of Wnt pathway signaling by a Wnt pathway inhibitor is a reduction in the level of Wnt pathway signaling of at least about 10%, at least about 25%, at least about 50%, at least about 75%, at least about 90%, or at least about 95%. In some embodiments, a Wnt pathway inhibitor that inhibits Wnt pathway signaling is an antibody. In some embodiments, a Wnt pathway inhibitor that inhibits Wnt pathway signaling is an anti-FZD antibody. In some embodiments, a Wnt pathway inhibitor that inhibits Wnt pathway signaling is antibody OMP-18R5.

In certain embodiments, the Wnt pathway inhibitor inhibits activation of β-catenin. It is understood that a Wnt pathway inhibitor that inhibits activation of β-catenin may, in certain embodiments, inhibit activation of β-catenin by one or more receptors, but not necessarily inhibit activation of β-catenin by all receptors. In certain alternative embodiments, activation of β-catenin by all human receptors may be inhibited. In certain embodiments, activation of β-catenin by one or more receptors selected from the group consisting of FZD1, FZD2, FZD3, FZD4, FDZ5, FDZ6, FDZ7, FDZ8, FDZ9, and FZD10 is inhibited. In certain embodiments, the inhibition of activation of 3-catenin by a Wnt-binding agent is a reduction in the level of activation of β-catenin of at least about 10%, at least about 25%, at least about 50%, at least about 75%, at least about 90%, or at least about 95%. In some embodiments, a Wnt pathway inhibitor that inhibits activation of β-catenin is an antibody. In some embodiments, a Wnt pathway inhibitor that inhibits activation of β-catenin is an anti-FZD antibody. In some embodiments, a Wnt pathway inhibitor that inhibits activation of β-catenin is antibody OMP-18R5.

In vivo and in vitro assays for determining whether a Wnt pathway inhibitor inhibits β-catenin signaling are known in the art. For example, cell-based, luciferase reporter assays utilizing a TCF/Luc reporter vector containing multiple copies of the TCF-binding domain upstream of a firefly luciferase reporter gene may be used to measure β-catenin signaling levels in vitro (Gazit et al., 1999, Oncogene, 18; 5959-66; TOPflash, Millipore, Billerica Mass.). The level of β-catenin signaling in the presence of one or more Wnt proteins (e.g., Wnt(s) expressed by transfected cells or provided by Wnt-conditioned media) in the presence of a binding agent is compared to the level of signaling without the binding agent present. In addition to the TCF/Luc reporter assay, the effect of a binding agent (or candidate agent) on μ-catenin signaling may be measured in vitro or in vivo by measuring the effect of the agent on the level of expression of β-catenin-regulated genes, such as c-myc, cyclin D1, and/or fibronectin. In certain embodiments, the effect of a binding agent on β-catenin signaling may also be assessed by measuring the effect of the agent on the phosphorylation state of Dishevelled-1, Dishevelled-2, Dishevelled-3, LRP5, LRP6, and/or β-catenin.

In certain embodiments, a Wnt pathway inhibitor has one or more of the following effects: inhibit proliferation of tumor cells, inhibit tumor growth, reduce the frequency of cancer stem cells in a tumor, reduce the tumorigenicity of a tumor, reduce the tumorigenicity of a tumor by reducing the frequency of cancer stem cells in the tumor, trigger cell death of tumor cells, induce cells in a tumor to differentiate, differentiate tumorigenic cells to a non-tumorigenic state, induce expression of differentiation markers in the tumor cells, prevent metastasis of tumor cells, or decrease survival of tumor cells.

In certain embodiments, a Wnt pathway inhibitor is capable of inhibiting tumor growth. In certain embodiments, a Wnt pathway inhibitor is capable of inhibiting tumor growth in vivo (e.g., in a xenograft mouse model, and/or in a human having cancer). In certain embodiments, the tumor is a lung tumor. In certain embodiments, the tumor is a Wnt-dependent tumor.

In certain embodiments, a Wnt pathway inhibitor is capable of reducing the tumorigenicity of a tumor. In certain embodiments, a Wnt pathway inhibitor is capable of reducing the tumorigenicity of a tumor comprising cancer stem cells in an animal model, such as a mouse xenograft model. In certain embodiments, the number or frequency of cancer stem cells in a tumor is reduced by at least about two-fold, about three-fold, about five-fold, about ten-fold, about 50-fold, about 100-fold, or about 1000-fold. In certain embodiments, the reduction in the number or frequency of cancer stem cells is determined by limiting dilution assay using an animal model. Additional examples and guidance regarding the use of limiting dilution assays to determine a reduction in the number or frequency of cancer stem cells in a tumor can be found, e.g., in International Publication No. WO 2008/042236 and U.S. Patent Publication Nos. 2008/0064049 and 2008/0178305.

In certain embodiments, a Wnt pathway inhibitor is active in vivo for at least 1 hour, at least about 2 hours, at least about 5 hours, at least about 10 hours, at least about 24 hours, at least about 2 days, at least about 3 days, at least about 1 week, or at least about 2 weeks. In certain embodiments, the Wnt pathway inhibitor is an IgG (e.g., IgG1 or IgG2) antibody that is active in vivo for at least 1 hour, at least about 2 hours, at least about 5 hours, at least about 10 hours, at least about 24 hours, at least about 2 days, at least about 3 days, at least about 1 week, or at least about 2 weeks. In certain embodiments, the Wnt pathway inhibitor is a fusion protein that is active in vivo for at least 1 hour, at least about 2 hours, at least about 5 hours, at least about 10 hours, at least about 24 hours, at least about 2 days, at least about 3 days, at least about 1 week, or at least about 2 weeks.

In certain embodiments, a Wnt pathway inhibitor has a circulating half-life in mice, cynomolgus monkeys, or humans of at least about 5 hours, at least about 10 hours, at least about 24 hours, at least about 2 days, at least about 3 days, at least about 1 week, or at least about 2 weeks. In certain embodiments, the Wnt pathway inhibitor is an IgG (e.g., IgG1 or IgG2) antibody that has a circulating half-life in mice, cynomolgus monkeys, or humans of at least about 5 hours, at least about 10 hours, at least about 24 hours, at least about 2 days, at least about 3 days, at least about 1 week, or at least about 2 weeks. Methods of increasing (or decreasing) the half-life of agents such as polypeptides and antibodies are known in the art. For example, known methods of increasing the circulating half-life of IgG antibodies include the introduction of mutations in the Fc region which increase the pH-dependent binding of the antibody to the neonatal Fc receptor (FcRn) at pH 6.0. Known methods of increasing the circulating half-life of antibody fragments lacking the Fc region include such techniques as PEGylation.

### IV. Kits

Kits for practicing the methods of the invention are further provided. By “kit” is intended any manufacture (e.g., a package or a container) comprising at least one reagent, e.g., an antibody, a nucleic acid probe, etc. for specifically detecting the expression of at least one biomarker of the invention. The kit may be promoted, distributed, and/or sold as a unit for performing the methods of the present invention. Additionally, the kits may contain a package insert describing the kit and including instructional material for its use.

In some embodiments, a kit comprises reagents for practicing the methods of the invention using microarray technology. In some embodiments, a kit comprises reagents for practicing the methods of the invention using qPCR assays. Positive and/or negative controls may be included in the kits to validate the activity and correct usage of reagents employed in accordance with the invention. Controls may include samples known to be either positive or negative for the presence of the biomarker of interest, or other samples comprising the biomarkers of interest. The design and use of controls is standard and well within the routine capabilities of those in the art.

In some embodiments, a kit comprises reagents for practicing the methods of the invention using an IHC assay. In some embodiments, a kit comprises an anti-LEF1. In some embodiments, the kits contain all of the components necessary and/or sufficient to perform an IHC assay, including all controls, directions for performing assays, and any necessary software for analysis and presentation of results.

It will be further appreciated that any or all steps in the methods of the invention could be implemented by personnel or, alternatively, performed in an automated fashion. Thus, the steps of sample preparation, detection of biomarker expression, etc. may be automated.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, may be practiced without departing from the scope of the present disclosure.

### EXAMPLES

### Example 1

Identification of Tumors Responsive to Treatment with OMP-18R5

The non-small cell lung cancer (NSCLC) tumor xenograft models OMP-LU02, OMP-LU11, OMP-LU24, OMP-LU25, OMP-LU33, OMP-LU45, and OMP-LU56 (and other OncoMed models described herein) were established at OncoMed Pharmaceuticals from minimally passaged, patient-derived tumor specimens. The tumor specimens were examined by a pathologist and classified as lung tumors. OncoMed relies on these classifications unless further analyses are done on any specific tumor and a reclassification is deemed necessary. Six- to 8-week-old NOD/SCID mice were subcutaneously injected with 5×10³ to 5×10⁴ cells of OMP-LU02, OMP-LU11, OMP-LU24, OMP-LU25, OMP-LU33, OMP-LU45, and OMP-LU56 tumors. Tumors were allowed to grow until they reached an average volume of 100 to 150 mm³. Tumor-bearing mice were randomized into groups (n=8-10 per group) and treated with control antibody or anti-FZD antibody OMP-18R5. The antibodies were dosed at 15-20 mg/kg and were administered intraperitoneally on a weekly basis. Tumor growth was monitored and tumor volumes were measured with electronic calipers at the indicated time points. Data are expressed as mean±S.E.M.

To determine if a tumor was responsive to anti-FZD antibody OMP-18R5, tumor volume data from mice treated with the control antibody were compared with tumor volume data from mice treated with OMP-18R5. For this study a “responder” tumor was defined as a tumor showing significantly greater tumor growth inhibition (TGI) after treatment with OMP-18R5 as compared to tumor growth inhibition after treatment with control antibody.

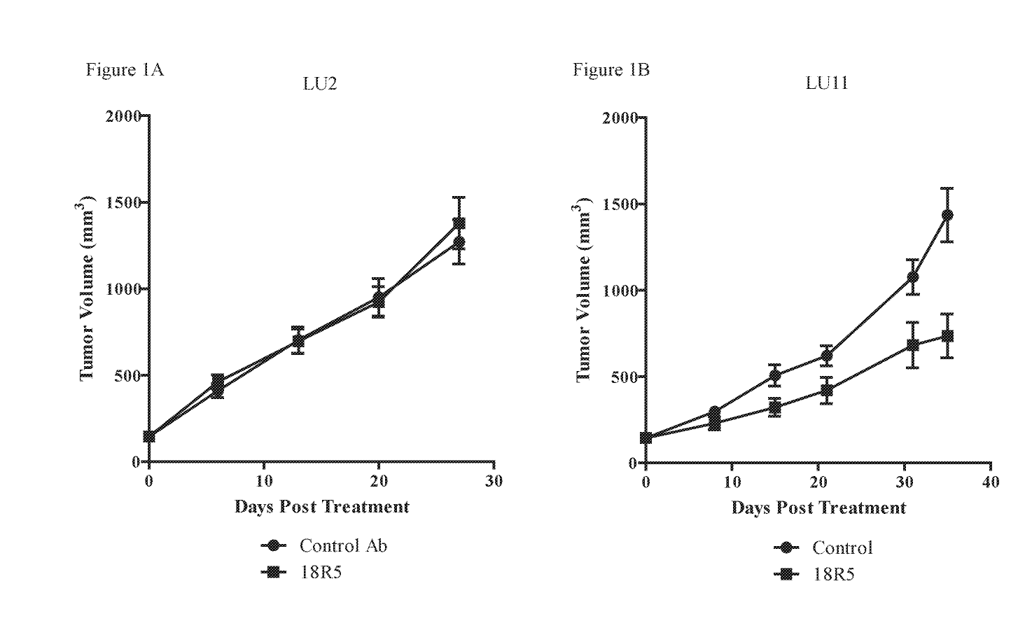

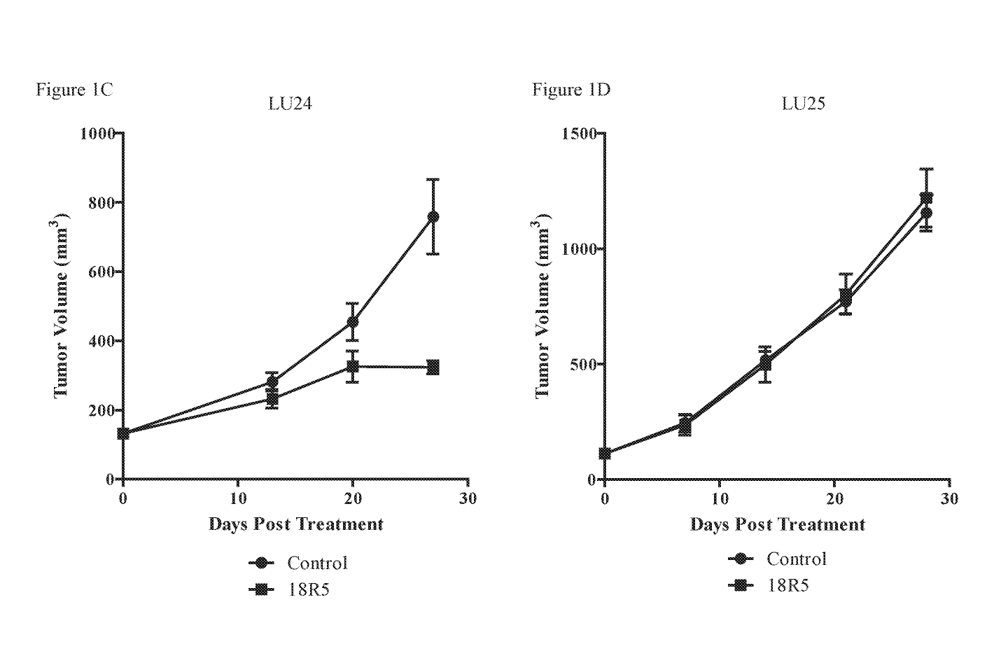

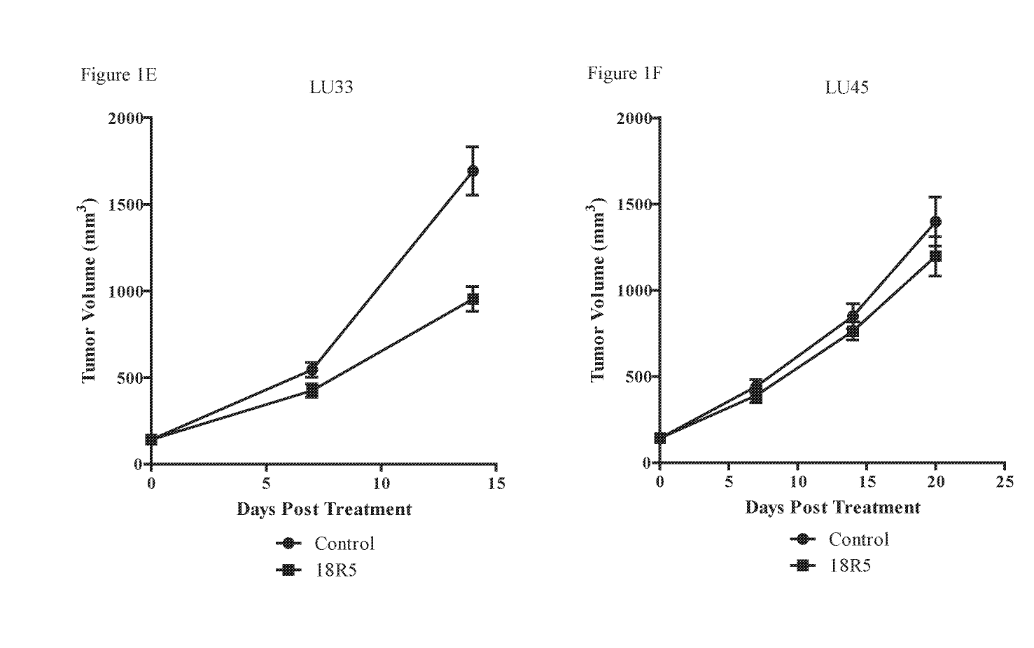

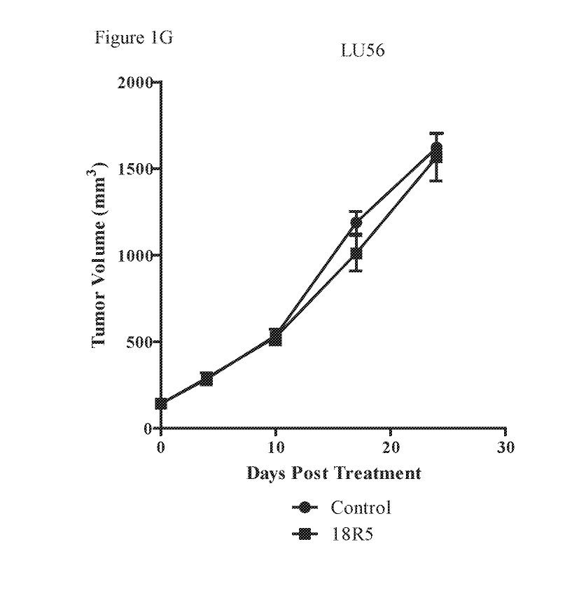

The results for each xenograft model are shown in FIGS. 1A-I G. The tumors OMP-LU11, OMP-LU24, and OMP-LU33 were shown to be responders, while tumors OMP-LU02, OMP-LU25, OMP-LU45, and OMP-LU56 were shown to be non-responders. The results are summarized in Table 1.

TABLE 1

| Tumor | Tumor Subtype | Classification |
|---|---|---|
| OMP-LU02 | Adenocarcinoma | Non-responder |
| OMP-LU11 | Squamous cell carcinoma | Responder |
| OMP-LU24 | Squamous cell carcinoma | Responder |
| OMP-LU25 | Large cell carcinoma | Non-responder |
| OMP-LU33 | Carcinoma | Responder |
| OMP-LU45 | Adenocarcinoma | Non-responder |
| OMP-LU56 | Carcinoma | Non-responder |

### Example 2

### Identification of Predictive Biomarkers

Microarray analyses were performed on untreated non-responder NSCLC tumors OMP-LU02, OMP-LU25, OMP-LU45, and OMP-LU56 and untreated responder tumors OMP-LU11, OMP-LU24, and OMP-LU33. RNA was isolated from the tumors using RNeasy Fibrous Tissue Mini Kits (Qiagen, Valencia Calif.) with DNAse treatment following the manufacturer's instructions. Samples were stored at −80° C. RNA was visualized on an Agilent 2100 Bioanalyzer and integrity was confirmed by the presence of intact 28S and 18S ribosomal peaks. All RNA samples had 260/280 ratios >1.8. Total RNA isolated from each tumor was amplified using the Ovation RNA Amplification System V2 (NuGEN, San Carlos, Calif.). Amplified, anti-sense single stranded-cDNA was fragmented and biotinylated using the FL-Ovation cDNA Biotin Module V2 (NuGEN). The quality of the cDNA and the fragmented cDNA was assessed by a spectrophotometer and a Bioanalyzer before hybridization to the array. The processed RNA was hybridized to Affymetrix HG-U133 plus 2.0 microarrays (Affymetrix, Santa Clara, Calif.) as outlined in the manufacturer's technical manuals. After hybridization, the microarrays were washed, scanned, and analyzed. Microarray data were processed to probe set level data by using GeneChip-RMA (Wu et al., 2004, J. Amer. Stat. Assn., 99:909-917). Probe sets that were likely to cross-hybridize with murine markers were removed. To summarize the data to gene level and make sure the probe set with the strongest signals were chosen, maximum expression was used across all probe sets mapping to one gene. Genes with low expression (<5 on log 2 scale) or near-zero variance (<0.01) were removed. Genes were standardized to N(0,1) by subtracting the log 2 scale expression from the mean and dividing by the standard deviation of each gene.

Analyses were focused on genes from Wnt-related pathways including canonical, planar cell polarity, Wnt/Ca+2, Wnt signaling negative regulation, cell fate, tissue polarity, cell growth and proliferation, cell migration, cell cycle, and cellular homeostasis (Table 2). Some genes were removed due to low variability or low expression and 79 unique genes were used in the analyses.

TABLE 2

| Gene Symbol | Protein Name |
|---|---|
| AES | Amino-terminal enhancer of split |
| APC | Adenomatous polyposis coli protein |
| AXIN1 | Axin-1 |
| BCL9 | B-cell CLL/lymphoma 9 protein |
| BTRC | F-box/WD repeat-containing protein 1A |
| CCND1 | G1/S-specific cyclin-D1 |
| CCND2 | G1/S-specific cyclin-D2 |
| CCND3 | G1/S-specific cyclin-D3 |
| CSNK1A1 | Casein kinase I isoform alpha |
| CSNK1D | Casein kinase I isoform delta |
| CSNK1G1 | Casein kinase I isoform gamma-1 |
| CSNK2A1 | Casein kinase II subunit alpha |
| CTBP1 | C-terminal-binding protein 1 |
| CTBP2 | C-terminal-binding protein 2 |
| CTNNB1 | Catenin beta-1 |
| CTNNBIP1 | Beta-catenin-interacting protein 1 |
| CXXC4 | CXXC-type zinc finger protein 4 |
| DAAM1 | Disheveled-associated activator of morphogenesis 1 |
| DIXDC1 | Dixin |
| DKK1 | Dickkopf-related protein 1 |
| DVL1 | Segment polarity protein disheveled homolog DVL-1 |
| DVL2 | Segment polarity protein disheveled homolog DVL-2 |
| EP300 | Histone acetyltransferase p300 |
| FBXW11 | F-box/WD repeat-containing protein 11 |
| FBXW2 | F-box/WD repeat-containing protein 2 |
| FBXW4 | F-box/WD repeat-containing protein 4 |
| FGF4 | Fibroblast growth factor 4 |
| FOSL1 | Fos-related antigen 1 |
| FOXN1 | Forkhead box protein N1 |
| FRAT1 | Proto-oncogene FRAT1 |
| FRZB | Secreted frizzled-related protein 3 |
| FSHB | Follitropin subunit beta |
| FZD1 | Frizzled-1 |
| FZD2 | Frizzled-2 |
| FZD3 | Frizzled-3 |
| FZD4 | Frizzled-4 |
| FZD5 | Frizzled-5 |
| FZD6 | Frizzled-6 |
| FZD7 | Frizzled-7 |
| FZD8 | Frizzled-8 |
| GSK3A | Glycogen synthase kinase-3 alpha |
| GSK3B | Glycogen synthase kinase-4 alpha |
| JUN | Transcription factor AP-1 |
| KREMEN1 | Kremen protein 1 |
| LEF1 | Lymphoid enhancer-binding factor 1 |
| LRP5 | Low-density lipoprotein receptor-related protein 5 |
| LRP6 | Low-density lipoprotein receptor-related protein 6 |
| MYC | Myc proto-oncogene protein |
| NKD1 | Protein naked cuticle homolog |
| NLK | Serine/threonine-protein kinase NLK |
| PITX2 | Pituitary homeobox 2 |
| PORCN | Protein-cysteine N-palmitoyl transferase porcupine |
| PPP2CA | Serine/threonine-protein phosphatase 2A catalytic |
| | subunit alpha isoform |
| PPP2R1A | Serine/threonine-protein phosphatase 2A 65 kDa |
| | regulatory subunit A alpha isoform |
| PYGO1 | Pygopus homolog 1 |
| RHOU | Rho-related GTP-binding protein RhoU |
| SENP2 | Sentrin-specific protease 2 |
| SFRP1 | Secreted frizzled-related protein 1 |
| SFRP4 | Secreted frizzled-related protein 4 |
| SLC9A3R1 | Na(+)/H(+) exchange regulatory cofactor NHE-RF1 |
| SOX17 | Transcription factor SOX-17 |
| T | Brachyury protein |
| TCF7 | Transcription factor 7 |
| TCF7L1 | Transcription factor 7-like 1 |
| TLE1 | Transducin-like enhancer protein 1 |
| TLE2 | Transducin-like enhancer protein 2 |
| WIF1 | Wnt inhibitory factor 1 |
| WISP1 | WNT1-inducible signaling pathway protein 1 |
| WNT1 | Proto-oncogene Wnt-1 |
| WNT2 | Protein Wnt-2 |
| WNT2B | Protein Wnt-2B |
| WNT3 | Protein Wnt-3 |
| WNT3A | Protein Wnt-3a |
| WNT4 | Protein Wnt-4 |
| WNT5A | Protein Wnt-5a |
| WNT5B | Protein Wnt-5b |
| WNT6 | Protein Wnt-6 |
| WNT7A | Protein Wnt-7a |
| WNT7B | Protein Wnt-7b |
| WNT8A | Protein Wnt-8a |
| WNT9A | Protein Wnt-9a |
| WNT10A | Protein Wnt-10a |
| WNT11 | Protein Wnt-11 |
| WNT16 | Protein Wnt-16 |

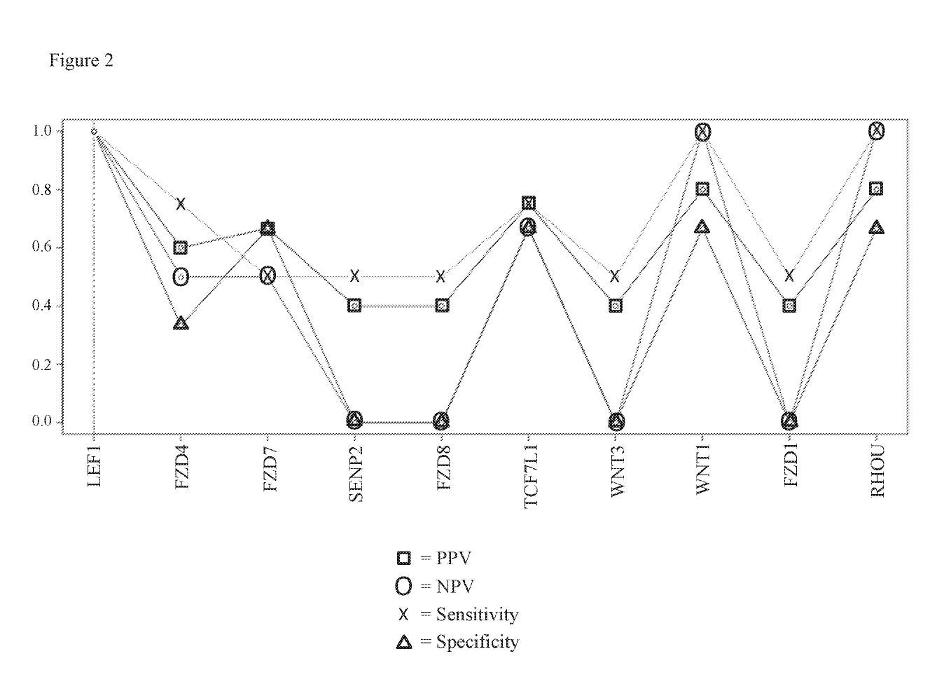

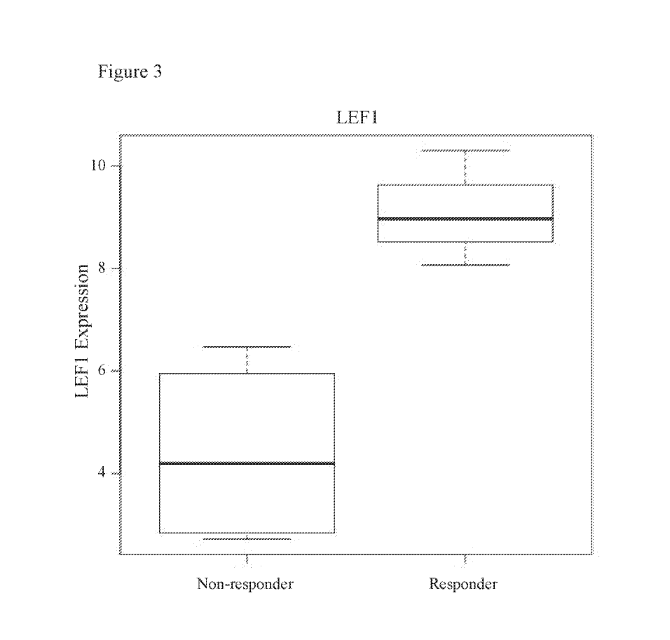

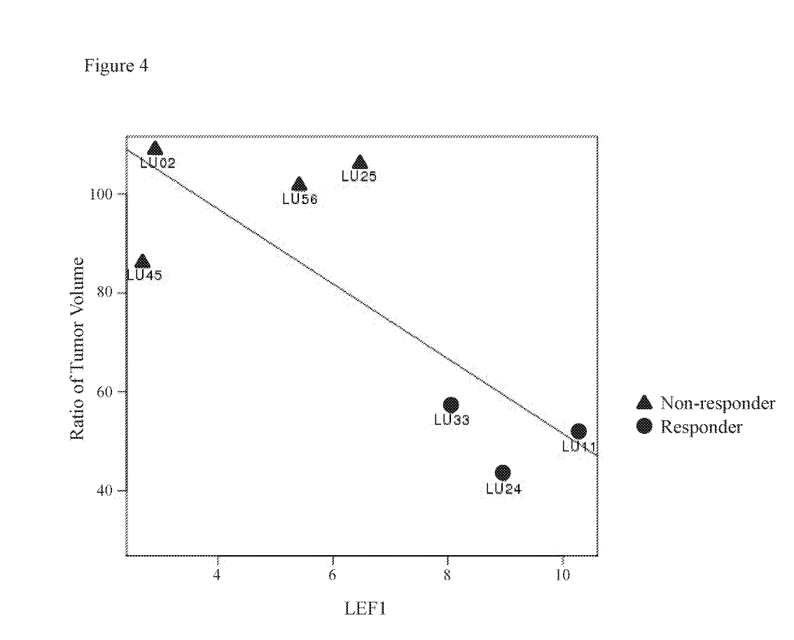

Support Vector Machines—Recursive Feature Elimination (SVM-RFE) methods (Guyon et al, 2002, Machine Learning, 46:389-422) were used to identify genes that could distinguish between the responder and non-responder tumors and Support Vector Machine (SVM) methods (Cortes and Vapnik, 1995, Machine Learning, 20:273-297) were used for classification. Leave-one-out cross-validation (LOOCV) was used to select the number of genes and also to measure positive predictive value (PPV), negative predictive value (NPV), sensitivity, and specificity of the models. A gene signature of LEF1 achieved the best performance with PPV=NPV=sensitivity=specificity=100% using the 7 lung tumors (FIG. 2). As shown in FIG. 3, the gene expression level of LEF1 was completely separated between responders and non-responders (p-value=0.009082). In addition, a strong correlation was observed between LEF1 gene expression and the ratio of tumor volume (RTV) from the in vivo experiments described in Example 1 (correlation=−0.79, p-value=0.036; FIG. 4). RTV was defined as the percentage of the average tumor volume of the OMP-18R5 treatment group over the average tumor volume of the control group at the last time point of the experiment.

Decision values were determined from the SVM model based on the training data. A positive decision value indicated a tumor predicted to be a responder while a negative decision value indicated a tumor predicted to be a non-responder. Classification probability can also be obtained by fitting a logistic regression on the decision values. Tumors associated with a probability higher than 0.5 would be predicted to be a responder while tumors with a probability lower than 0.5 would be predicted to be a non-responder.

### Example 3

### In Vivo Validation of Predictive Biomarker LEF1

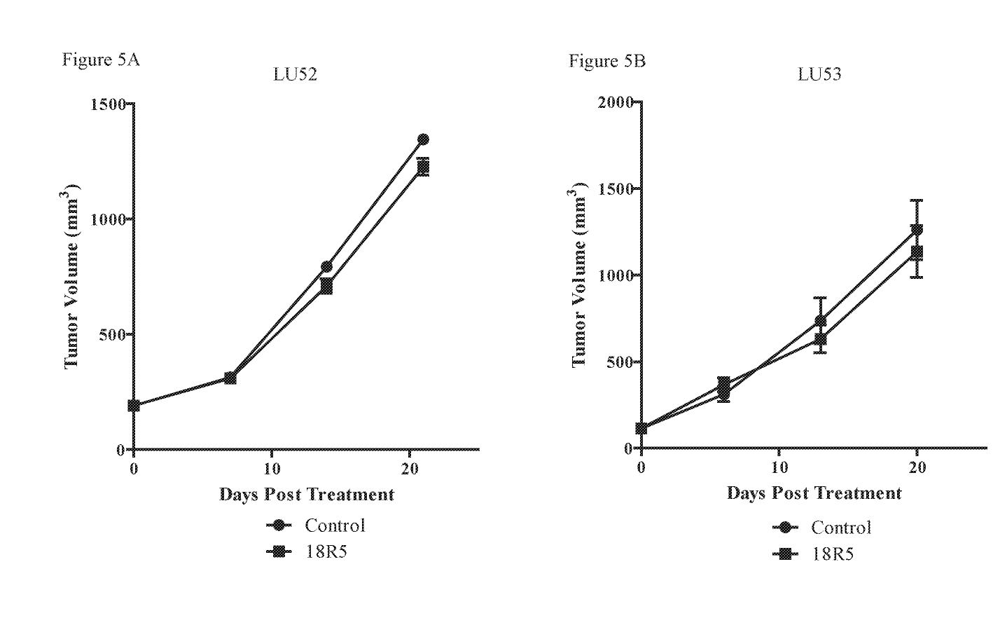

Two additional NSCLC tumors (OMP-LU52 and OMP-LU53) were selected from the OncoMed Tumor Bank and microarray analyses were performed as described in Example 1. The LEF1 biomarker was used to predict the response of these tumors to treatment with anti-FZD antibody OMP-18R5. In parallel the two tumors were evaluated in in vivo xenograft models as described in Example 1 (see FIGS. 5A-5B). As described in Example 1 a “responder” in the in vivo model is a tumor showing significantly greater tumor growth inhibition after treatment with OMP-18R5 as compared to tumor growth inhibition after treatment with the control antibody. The predictions were compared to the results of the in vivo xenograft models. The results are shown in Table 3.

TABLE 3

| | | Proba- | Decision | | In vivo |
|---|---|---|---|---|---|
| Tumor | Tumor Subtype | bility | Value | Prediction | Response |
| OMP- | Large cell | 0.21 | −3.48 | Non- | Non- |
| LU52 | neuroendocrine | | | responder | responder |
| | carcinoma | | | | |
| OMP- | Squamous cell | 0.14 | −4.73 | Non- | Non- |
| LU53 | carcinoma | | | responder | responder |

As shown in Table 3, the response of each of the two NSCLC tumors was accurately predicted by the LEF1 biomarker.

### Example 4

Correlation of LEF1 Expression to Response of NSCLC Tumors to 18R5 in Combination with Chemotherapy

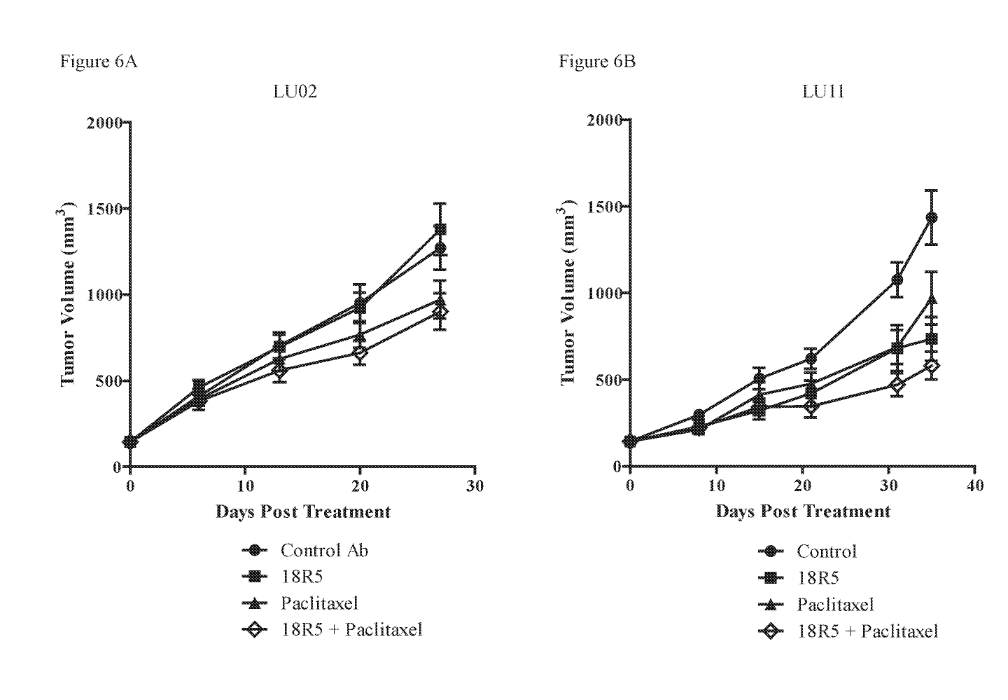

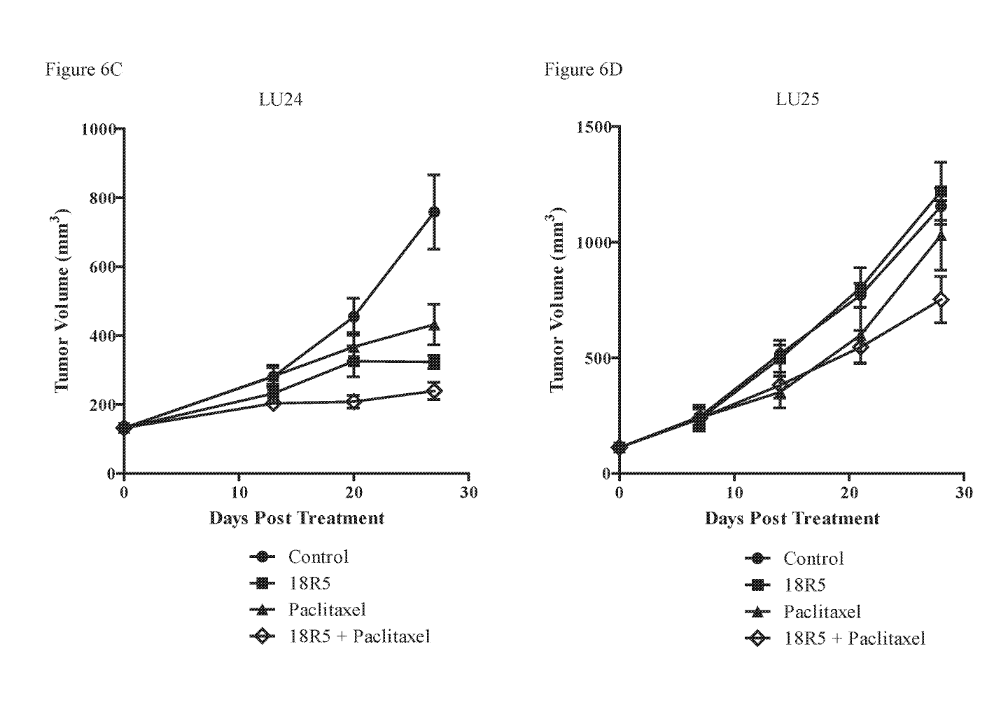

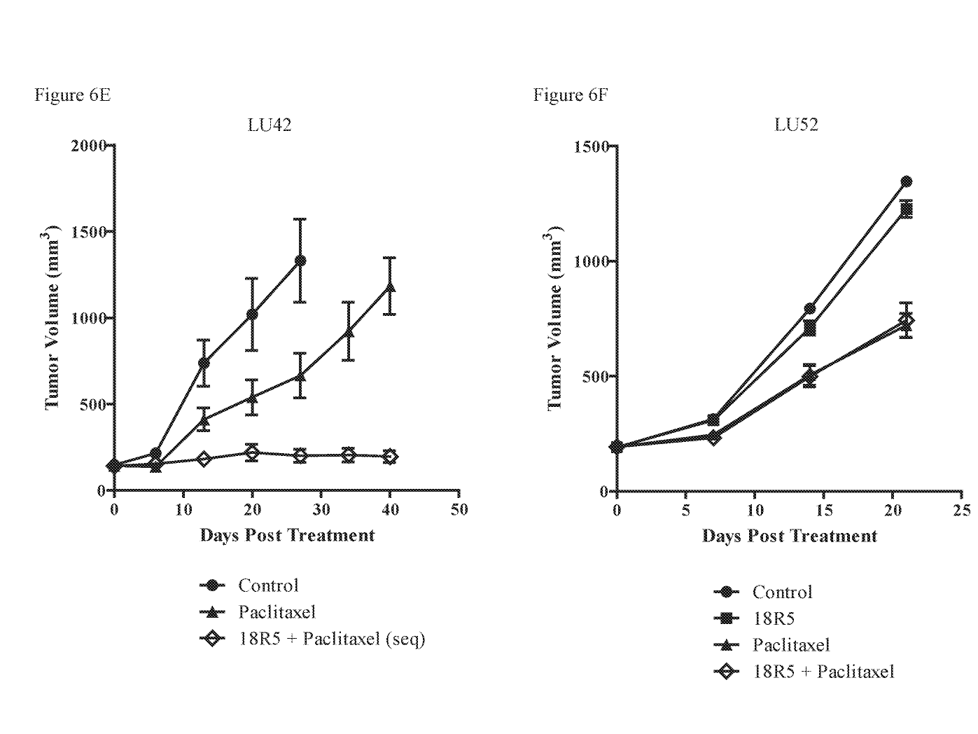

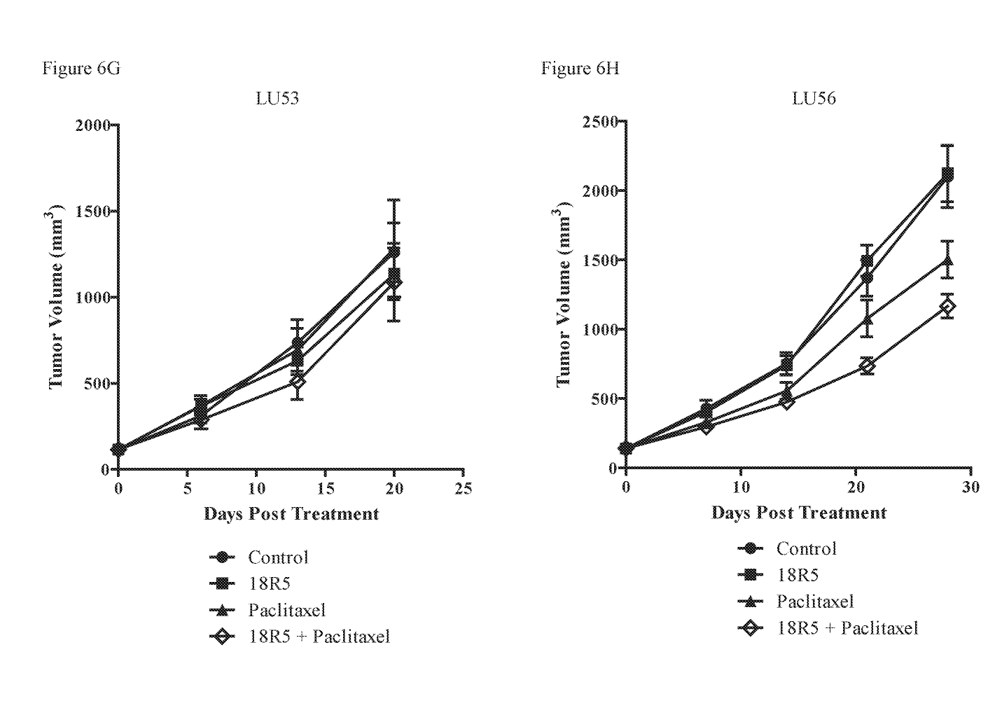

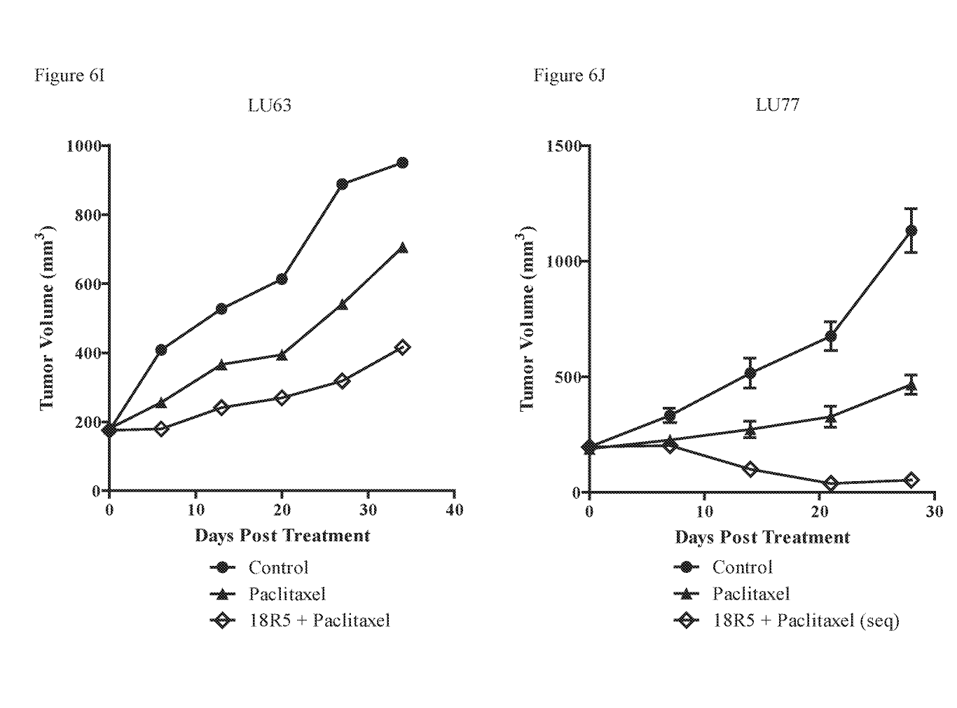

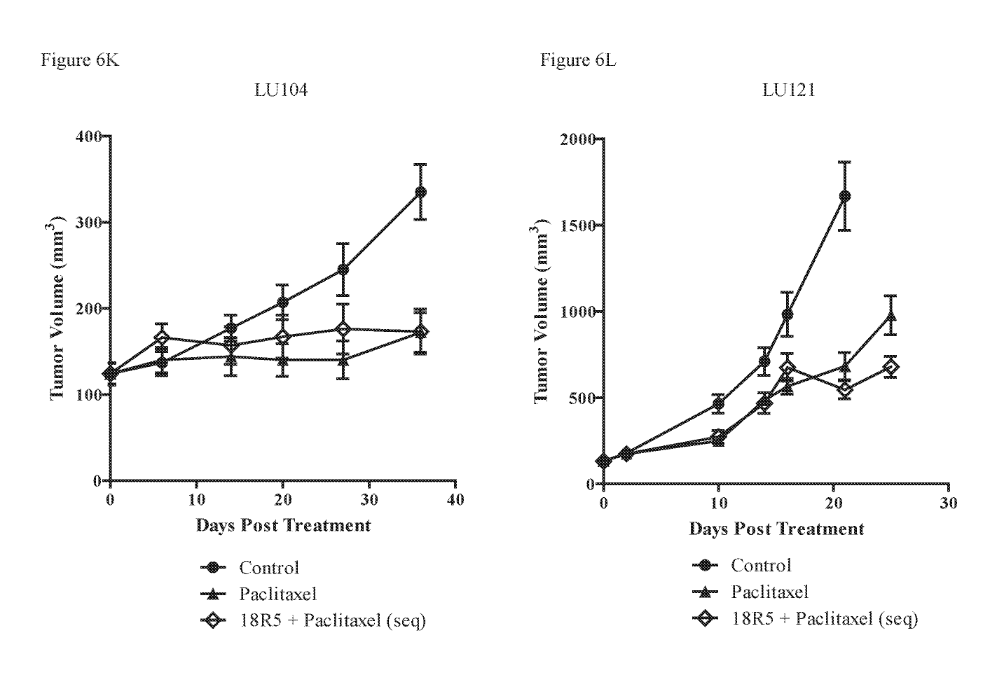

To assess if LEF1 gene expression correlates with the response to OMP-18R5 in combination with chemotherapy, the in vivo experimental design described in Example 1 was modified to include groups of mice treated with paclitaxel as a single agent or treated with OMP-18R5 in combination with paclitaxel. Five additional NSCLC xenograft tumors were selected from the OncoMed Tumor Bank and submitted for microarray analysis. The tumors were OMP-LU42, OMP-LU63, OMP-LU77, OMP-LU104, and OMP-LU121. These five tumors and seven of the tumors described in Examples 1 and 3 were evaluated for their response to OMP-18R5 in combination with the paclitaxel. Six- to 8-week-old NOD/SCID mice were subcutaneously injected with 5×10³ to 5×10⁴ cells of OMP-LU02. OMP-LU11, OMP-LU24. OMP-LU25, OMP-LU42, OMP-LU52, OMP-LU53, OMP-LU56, OMP-LU63, OMP-LU77, OMP-LU104, and OMP-LU121 tumors. Tumors were allowed to grow until they reached an average volume of between 100 and 150 mm³. Tumor-bearing mice were randomized into three or four groups (n=8-10 per group) and treated with anti-FZD antibody 18R5 alone (20-25 mg/kg), anti-FZD antibody OMP-18R5 (20-25 mg/kg) in combination with paclitaxel (10-20 mg/kg), paclitaxel alone (10-20 mg/kg), or control antibody (20-25 mg/kg). OMP-18R5 and paclitaxel were administered intraperitoneally once a week (OMP-LU02, OMP-LU11, OMP-LU24, OMP-LU25, OMP-LU52, OMP-LU53, OMP-LU56, and OMP-LU63) or every other week (OMP-LU42, OMP-LU77, OMP-LU104, and OMP-LU121). When paclitaxel was dosed every other week, it was administered 48 hour after OMP-18R5 (sequential dosing). Tumor growth was monitored and tumor volumes were measured with electronic calipers at the indicated time points. Data are expressed as mean±S.E.M. The results of these experiments are shown in FIGS. 6A-6L.

For this study a “responder” tumor was defined as a tumor showing significantly greater tumor growth inhibition after treatment with OMP-18R5 in combination with paclitaxel as compared to tumor growth inhibition after treatment with paclitaxel alone. The results are summarized in Table 4.

TABLE 4

| Tumor | Tumor Subtype | In vivo Response |
|---|---|---|
| OMP-LU02 | Adenocarcinoma | Non-responder |
| OMP-LU11 | Squamous cell carcinoma | Responder |
| OMP-LU24 | Squamous cell carcinoma | Responder |
| OMP-LU25 | Large cell carcinoma | Responder |
| OMP-LU42 | Squamous cell carcinoma | Responder |
| OMP-LU52 | Large cell neuroendocrine carcinoma | Non-responder |
| OMP-LU53 | Squamous cell carcinoma | Non-responder |
| OMP-LU56 | Carcinoma | Responder |
| OMP-LU63 | Large cell neuroendocrine carcinoma | Responder |
| OMP-LU77 | Adenosquamous cell carcinoma | Responder |
| OMP-LU104 | Adenocarcinoma | Non-responder |
| OMP-LU121 | Adenocarcinoma | [symbol] |
| | | Non-responder |

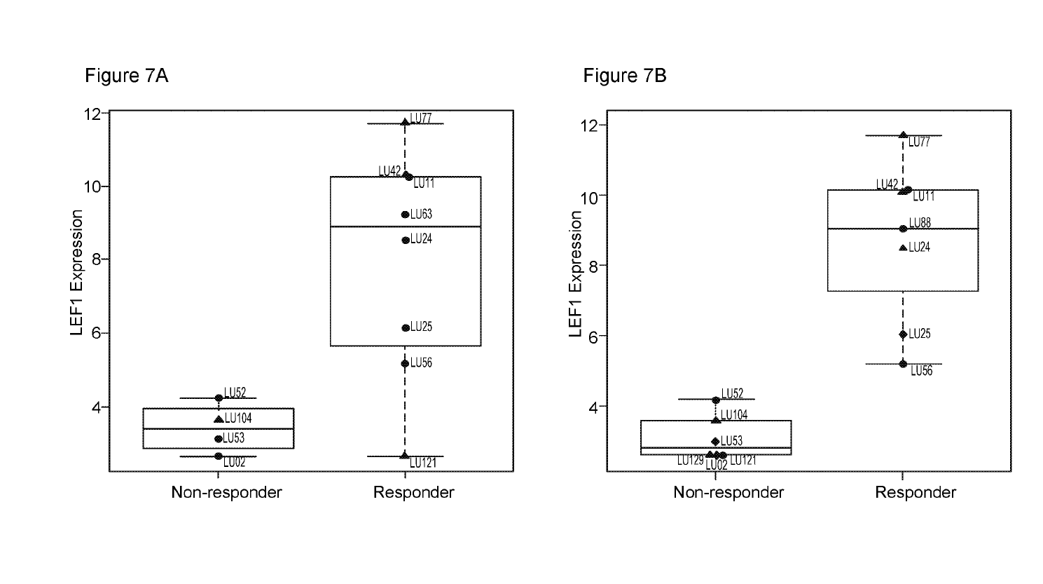

The LEF1 biomarker was significantly differentially expressed between responders and non-responders (p-value=0.0162) (FIG. 7A). The gene expression level of LEF1 was higher in the majority of the responders, and lower in all the non-responders.

In subsequent experiments, lung tumor OMP-LU121 was reclassified as a non-responder and LEF1 expression was reanalyzed (FIG. 7B). The gene expression level of LEF1 was higher in all of the responders, and lower in all the non-responders.

In addition, a pharmacodynamics (PD) biomarker analysis was performed and confirmed inhibition of genes in the Wnt. Notch, and stem cell pathways by treatment with OMP-18R5. Wnt pathway target genes including AXIN2 and LEF1 were down-regulated significantly by treatment with OMP-18R5 both as a single agent and in combination with paclitaxel (data not shown).

### Example 5

### Prevalence Estimation of the LEF1 Biomarker

Prevalence of a biomarker signature can be defined as the proportion of a population predicted to be a responder based upon the biomarker signature. The prevalence of the LEF1 biomarker in NSCLC populations was estimated by applying the LEF1 biomarker to three publicly available NSCLC microarray data sets. The Bild2006 dataset was compiled from Affymetrix U133 Plus 2 microarrays with 111 NSCLC patients including 54 squamous cell carcinoma samples and 57 adenocarcinoma samples. The Yamauchi2012 dataset was compiled from Affymetrix U133 Plus 2 microarrays with 226 lung adenocarcinoma patients. The Raponi2006 dataset was compiled from Affymetrix U133 Plus 2 microarrays with 130 squamous cell carcinoma patients. Pre-processing of the public data included downloading the data, extracting the probe sets mapping to the LEF1 gene, and collapsing the probe sets to one gene. Gene level expression data was further standardized by subtracting the mean and dividing by the standard deviation of each gene in the public data. The SVM model built upon the training data was used to classify the public data. Classification probabilities were obtained and the proportion of predicted responders (probability>0.5) was calculated based on the LEF1 biomarker.

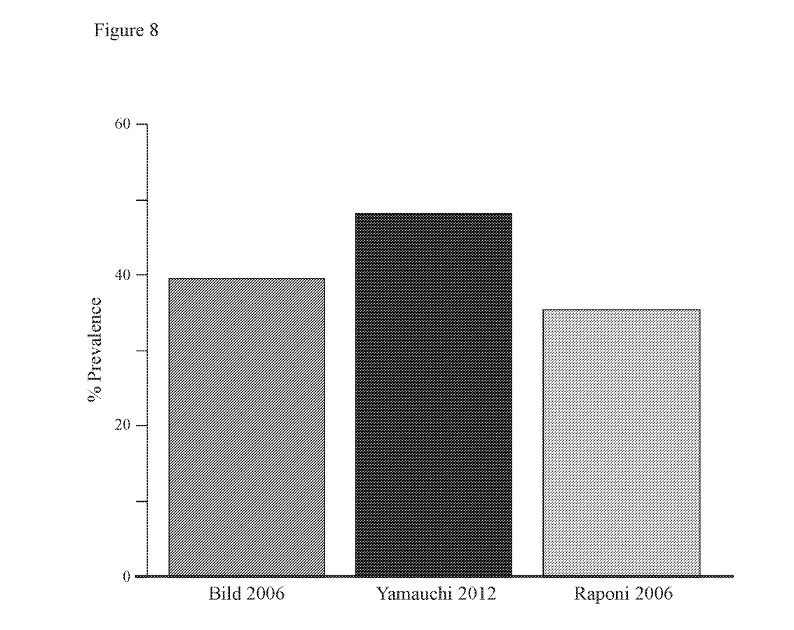

The predicted prevalence of the LEF1 biomarker within the three datasets ranged from 35% to 50% (FIG. 8). This prediction would suggest that there is a large population of NSCLC patients that would be responsive to therapy with the anti-FZD antibody OMP-18R5.

It is understood that the examples and embodiments described herein are for illustrative purposes only and that various modifications or changes in light thereof will be suggested to persons skilled in the art and are to be included within the spirit and purview of this application.

All publications, patents, and patent applications cited herein are hereby incorporated by reference in their entirety for all purposes to the same extent as if each individual publication, patent or patent application were specifically and individually indicated to be so incorporated by reference.

Following are the sequences disclosed in the application:

OMP-18R5 Heavy chain CDR1
(SEQ ID NO: 1)
GFTFSHYTLS
OMP-18R5 Heavy chain CDR2
(SEQ ID NO: 2)
VISGDGSYTYYADSVKG
OMP-18R5 Heavy chain CDR3
(SEQ ID NO: 3)
NFIKYVFAN
OMP-18R5 Light chain CDR1
(SEQ ID NO: 4)
SGDNIGSFYVH
OMP-18R5 Light chain CDR2
(SEQ ID NO: 5)
DKSNRPSG
OMP-18R5 Light chain CDR3
(SEQ ID NO: 6)
QSYANTLSL
OMP-18R5 Heavy chain variable region amino acid
sequence
(SEQ ID NO: 7)
EVQLVESGGGLVQPGGSLRLSCAASGFTFSHYTLSWVRQAPGKGLEWVSV
ISGDGSYTYYADSVKGRFTISSDNSKNTLYLQMNSLRAEDTAVYYCARNF
IKYVFANWGQGTLVTVSS
OMP-18R5 Light chain variable region amino acid
sequence
(SEQ ID NO: 8)
DIELTQPPSVSVAPGQTARISCSGDNIGSFYVHWYQQKPGQAPVLVIYDK
SNRPSGIPERFSGSNSGNTATLTISGTQAEDEADYYCQSYANTLSLVFGG
GTKLTVLG
OMP-18R5 Heavy chain amino acid sequence with pre-
dicted signal sequence underlined
(SEQ ID NO: 9)
MKHLWFFLLLVAAPRWVLSEVQLVESGGGLVQPGGSLRLSCAASGFTFSH
YTLSWVRQAPGKGLEWVSVISGDGSYTYYADSVKGRFTISSDNSKNTLYL
QMNSLRAEDTAVYYCARNFIKYVFANWGQGTLVTVSSASTKGPSVFPLAP
CSRSTSESTAALGCLVKDYFPEPVTVSWNSGALTSGVHTFPAVLQSSGLY
SLSSVVTVPSSNFGTQTYTCNVDHKPSNTKVDKTVERKCCVECPPCPAPP
VAGPSVFLFPPKPKDTLMISRTPEVTCVVVDVSHEDPEVQFNWYVDGVEV
HNAKTKPREEQFNSTFRVVSVLTVVHQDWLNGKEYKCKVSNKGLPAPIEK
TISKTKGQPREPQVYTLPPSREEMTKNQVSLTCLVKGFYPSDIAVEWESN
GQPENNYKTTPPMLDSDGSFFLYSKLTVDKSRWQQGNVFSCSVMHEALHM
HYTQKSLSLSPGK
OMP-18R5 Light chain amino acid sequence with pre-
dicted signal sequence underlined
(SEQ ID NO: 10)
MAWALLLLTLLTQGTGSWADIELTQPPSVSVAPGQTARISCSGPNIGSFY
VHWYQQKFGQAPVLVIYDKSNRPSGIPERFSGSNSGNTATLTISGTQAED
EADYYCQSYANTLSLVFGGGTKLTVLGQPKAAPSVTLFPPSSEELQANKA
TLVCLISDFYPGAVTVAWKADSSPVKAGVETTTPSKQSNNKYAASSYLSL
TPEQWKSHRSYSCQVTKEGSTVEKTVAPTECS
OMP-18R5 Heavy chain amino acid sequence without
predicted signal sequence
(SEQ ID NO: 11)
EVQLVESGGGLVQPGGSLRLSCAA5GFTFSHYTLSWVRQAPGKGLEWVSV
ISGDGSYTYYADSVKGRFTISSDNSKNTLYLQMNSLRAEDTAVYYCARNF
IKYVFAKWGQGTLVTVSSASTKGPSVFPLAPCSRSTSESTAALGCLVKDY
FPEPVTVSWNSGALTSGVHTFPAVLQSSGLYSLSSVVTVPSSNFGTQTYT
CNVDHKPSNTKVDKTVERKCCVECPPCPAPPVAGPSVFLFPPKPKDTLMI
SRTPEVTCVVVDVSHEDPEVQFNWYVDGVEVHNAKTKPREEQFNSTFRVV
SVLTVVHQDWLNGKEYKCKVSNKGLPAPIEKTISKTKGQPREPQVYTLPP
SREEMTKNQVSLTCLVKGFYPSDIAVEWESNGQPENNYKTTPPMLDSDGS
FFLYSKLTVDKSRWQQGNVFSCSVMHEALHMHYTQKSLSLSPGK
OMP-18R5 Light chain amino acid sequence without
predicted signal sequence
(SEQ ID NO: 12)
DIELTQPPSVSVAPGQTARISCSGDNIGSFYVHWYQQKPGQAPVLVIYDK
SNRPSGIPERFSGSNSGNTATLTISGTQAEDEADYYCQSYANTLSLVFGG
GTKLTVLGQPKAAPSVTLFPPSSEELQANKATLVCLISDFYPGAVTVAWK
ADSSPVKAGVETTTPSKQSNNKYAASSYLSLTPEQWKSHRSYSCQVTHEG
STVEKTVAPTECS
FLAG peptide
(SEQ ID NO: 13)
DYKDDDDK
Human LEF1 nucleotide sequence
(SEQ ID NO: 14)
ATGCCCCAACTCTCCGGAGGAGGTGGCGGCGGCGGGGGGGACCCGGAACT
CTGCGCCACGGACGAGATGATCCCCTTCAAGGACGAGGGCGATCCTCAGA
AGGAAAAGATCTTCGCCGAGATCAGTCATCCCGAAGAGGAAGGCGATTTA
GCTGACATCAAGTCTTCCTTGGTGAACGAGTCTGAAATCATCCCGGCCAG
CAACGGACACGAGGTGGCCAGACAAGCACAAACCTCTCAGGAGCCCTACC
ACGACAAGGCCAGAGAACACCCCGATGACGGAAAGCATCCAGATGGAGGC
CTCTACAACAAGGGACCCTCCTACTCGAGTTATTCCGGGTACATAATGAT
GCCAAATATGAATAACGACCCATACATGTCAAATGGATCTCTTTCTCCAC
CCATCCCGAGAACATCAAATAAAGTGCCCGTGGTGCAGCCATCCCATGCG
GTCCATCCTCTCACCCCCCTCATCACTTACAGTGACGAGCACTTTTCTCC
AGGATCACACCCGTCACACATCCCATCAGATGTCAACTCCAAACAAGGCA
TGTCCAGACATCCTCCAGCTCCTGATATCCCTACTTTTTATCCCTTGTCT
CCGGGTGGTGTTGGACAGATCACCCCACCTCTTGGCTGGCAAGGTCAGCC
TGTATATCCCATCACGGGTGGATTCAGGCAACCCTACCCATCCTCACTGT
CAGTCGACACTTCCATGTCCAGGTTTTCCCATCATATGATTCCCGGTCCT
CCTGGTCCCCACACAACTGGCATCCCTCATCCAGCTATTGTAACACCTCA
GGTCAAACAGGAACATCCCCACACTGACAGTGACCTAATGCACGTGAAGC
CTCAGCATGAACAGAGAAAGGAGCAGGAGCCAAAAAGACCTCACATTAAG
AAGCCTCTGAATGCTTTTATGTTATACATGAAAGAAATGAGAGCGAATGT
CGTTGCTGAGTGTACTCTAAAAGAAAGTGCAGCTATCAACCAGATTCTTG
GCAGAAGGTGGCATGCCCTCTCCCGTGAAGAGCAGGCTAAATATTATGAA
TTAGCACGGAAAGAAAGACAGCTACATATGCAGCTTTATCCAGGCTGGTC
TGCAAGAGACAATTATGGTAAGAAAAAGAAGAGGAAGAGAGAGAAACTAC
AGGAATCTGCATCAGGTACAGGTCCAAGAATGACAGCTGCCTACATCTGA
Human LEF1 amino acid sequence
(SEQ ID NO: 15)
MPQLSGGGGGGGGDPELCATDEMIPFKDEGDPQKEKIFAEISHPEEEGDL
ADIKSSLVNESEIIPASNGHEVARQAQTSQEPYHDKAREHPDDGKHPDGG
LYNKGPSYSSYSGYIMMPNMNNDPYMSNGSLSPPIPRTSNKVPVVQPSHA
VHPLTPLITYSDEHFSPGSHPSHIPSDVNSKQGMSRHPPAPDIPTFYPLS
PGGVGQITPPLGWQGQPVYPITGGFRQPYPSSLSVDTSMSRFSHHMIPGP
PGPHTTGIPHPAIVTPQVKQEHPHTDSDLMHVKPQHEQRKEQEPKRPHIK
KPLNAFMLYMKEMRANVVAECTLKESAAINQILGRRWHALSREEQAKYYE
LARKERQLHMQLYPGWSARDNYGKKKKRKREKLQESASGTGPRMTAAYI
Human FZD8 Fri domain amino acid sequence
(SEQ ID NO: 16)
ASAKELACQEITVPLCKGIGYNYTYMPNQFNHDTQDEAGLEVHQFWPLVE
IQCSPDLKFFLCSMYTPICLEDYKKPLPPCRSVCERAKAGCAPLMRQYGF
AWPDRMRCDRLPEQGNPDTLCMDYNRTDLTT
Human IgG₁ Fc region
(SEQ ID NO: 17)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK
Human IgG₁ Fc region (variant)
(SEQ ID NO: 18)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGK
Human IgG₁ Fc region
(SEQ ID NO: 19)
KSSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Human IgG₁ Fc region
(SEQ ID NO: 20)
EPKSSDKTHTCPPCPAPELLGGPSVFLFPPKPKDTLMISRTPEVTCVVVD
VSHEDPEVKFNWYVDGVEVHMAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK
Human IgG₁ Fc region
(SEQ ID NO: 21)
CVECPPCPAPPVAGPSVFLFPPKPKDTLMISRTPEVTCVVVDVSHEDPEV
QFNWYVDGVEVHNAKTKPREEQFNSTFRVVSVLTVVHQDWLNGKEYKCKV
SNKGLPAPIEKTISKTKGQPREPQVYTLPPSREEMTKNQVSLTCLVKGFY
PSDIAVEWESNGQPENNYKTTPPMLDSDGSFFLYSKLTVDKSRWQQGNVF
SCSVMHEALKNHYTQKSLSLSPGK
FZD8-Fc 54F28 amino acid sequence (without
predicted signal sequence)
(SEQ ID NO: 22)
ASAKELACQEITVPLCKGIGYNYTYMPNQFNHDTQDEAGLEVHQFWPLVE
IQCSPDLKFFLCSMYTPICLEDYKKPLPPCRSVCERAKAGCAPLMRQYGF
AWPDRMRCDRLPEQGNPDTLCMDYNRTDLTTEPKSSDKTHTCPPCPAPEL
LGGPSVFLFPPKPKDTLMISRTPEVTCVVVDVSHEDPEVKFNWYVDGVEV
HNAKTKPREEQYNSTYRVVSVLTVLKQDWLNGKEYKCKVSNKALPAPIEK
TISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHN
HYTQKSLSLSPGK
FZD8-Fc 54F28 with predicted signal sequence
underlined
(SEQ ID NO: 23)
KEWGYLLEVTSLLAALLLLQKSPFVHAASAKELACQEITVPLCKGIGYNY
TYMPNQFNHDTQDEAGLEVHQFWPLVEIQCSPDLKFFLCSMYTPICLEDY
KKPLPPCRSVCERAKAGCAPLMRQYGFAWPDRMRCDRLPEQGNPDTLCMD
YNRTDLTTEPKSSDKTHTCPPCPAPELLGGPSVFLFPPKPKDTLMISRTP
EVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLY
SKLTVDKSRWQQGNVFSCSVMHEALHNHYTQKSLSLSPGK

What is claimed is:

1. A method of identifying a human lung tumor that is likely to be responsive or non-responsive to treatment with a Wnt pathway inhibitor, the method comprising:
(a) obtaining a sample of the human lung tumor;
(b) measuring the expression level of a biomarker signature in the sample, wherein the biomarker signature comprises LEF1; and
(c) identifying the lung tumor as likely to be responsive or non-responsive to treatment based upon the expression level of the biomarker signature.

2. A method of classifying a human lung tumor as likely to be responsive or non-responsive to treatment with a Wnt pathway inhibitor, the method comprising:
(a) obtaining a sample of the lung tumor;
(b) measuring the expression level of a biomarker signature in the sample, wherein the biomarker signature comprises LEF1; and
(c) classifying the lung tumor as likely to be responsive or non-responsive to treatment based upon the expression level of the biomarker signature.

3. A method of determining the responsiveness of a human lung tumor to treatment with a Wnt pathway inhibitor, the method comprising:
(a) obtaining a sample of the lung tumor;
(b) measuring the expression level of a biomarker signature in the sample, wherein the biomarker signature comprises LEF1; and
(c) determining the responsiveness of the lung tumor to treatment based upon the expression level of the biomarker signature.

4. A method of identifying a patient with lung cancer who is likely to respond to treatment with a Wnt pathway inhibitor, the method comprising:
(a) obtaining a sample of the patient's lung tumor;
(b) measuring the expression level of a biomarker signature in the sample, wherein the biomarker signature comprises LEF1; and
(c) identifying the patient who is likely to respond to treatment based upon the expression level of the biomarker signature.

5. A method of selecting a patient with lung cancer for treatment with a Wnt pathway inhibitor, the method comprising:
(a) obtaining a sample of the patient's lung cancer;
(b) measuring the expression level of a biomarker signature in the sample, wherein the biomarker signature comprises LEF1; and
(c) selecting the patient for treatment based upon the expression level of the biomarker signature.

6. A method of treating lung cancer in a patient, comprising:
(a) identifying if the patient is likely to respond to treatment with a Wnt pathway inhibitor, wherein the identification comprises:
(i) obtaining a sample of the patient's lung cancer;
(ii) measuring the expression level of a biomarker signature in the sample, wherein the biomarker signature comprises LEF1; and
(iii) identifying the patient who is likely to respond to treatment based upon the expression level of the biomarker signature; and
(b) administering a therapeutically effective amount of a Wnt pathway inhibitor to the patient who is likely to respond to treatment.

7. A method of treating lung cancer in a patient, comprising: administering a therapeutically effective amount of a Wnt pathway inhibitor to the patient; wherein the patient is predicted to respond to treatment with the Wnt pathway inhibitor based upon expression levels of a biomarker signature in a sample of the patient's lung cancer, wherein the biomarker signature comprises LEF1.

8. A method for increasing the likelihood of effective treatment with a Wnt pathway inhibitor, comprising:
(a) identifying if a patient has an lung tumor that is likely to respond to treatment with a Wnt pathway inhibitor, wherein the identification comprises:
(i) obtaining a sample of the patient's lung cancer;
(ii) measuring the expression level of a biomarker signature in the sample, wherein the biomarker signature comprises LEF1; and
(iii) identifying the patient who is likely to respond to treatment based upon the expression level of the biomarker signature; and
(b) administering a therapeutically effective amount of the Wnt pathway inhibitor to the patient who is likely to respond to treatment.

9. A method for increasing the likelihood of effective treatment with a Wnt pathway inhibitor comprising: administering a therapeutically effective amount of a Wnt pathway inhibitor to a patient; wherein the patient is identified as likely to respond to treatment with the Wnt pathway inhibitor based upon expression levels of a biomarker signature in a sample of the patient's lung tumor, wherein the biomarker signature comprises LEF1.

10. The method of any one of claims 1-9, wherein the biomarker signature is LEF1.

11. The method of any one of claims 1-10, wherein the expression of the biomarker signature is measured by a PCR-based assay.

12. The method of claim 11, wherein the expression of the biomarker signature is measured by a qPCR assay.

13. The method of any one of claims 1-10, wherein the expression of the biomarker signature is measured by a microarray.

14. The method of any one of claims 1-10, wherein the expression of the biomarker signature is determined by RNA sequencing.

15. The method of any one of claims 1-10, wherein the expression of the biomarker signature is determined by an immunohistochemistry assay.

16. The method of any one of claims 1-15, wherein the Wnt pathway inhibitor is an antibody.

17. The method of claim 16, wherein the Wnt pathway inhibitor is an antibody that specifically binds at least one human Frizzled (FZD) protein or fragment thereof.

18. The method of claim 17, wherein the antibody specifically binds at least one FZD protein selected from the group consisting of: FZD1, FZD2, FZD3, FZD4, FZD5, FZD6, FZD7, FZD8, FZD9, and FZD10.

19. The method of claim 17, wherein the antibody specifically binds at least one FZD protein selected from the group consisting of: FZD1, FZD2, FZD5, FZD7, and FZD8.

20. The method of claim 17, wherein the antibody specifically binds FZD1, FZD2, FZD5, FZD7, and FZD8.

21. The method of any one of claims 1-20, wherein the Wnt pathway inhibitor is an antibody comprising:
(a) a heavy chain CDR1 comprising GFTFSHYTLS (SEQ ID NO: 1), a heavy chain CDR2 comprising VISGDGSYTYYADSVKG (SEQ ID NO:2), and a heavy chain CDR3 comprising NFIKYVFAN (SEQ ID NO:3), and
(b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6).

22. The method of claim 21, wherein the Wnt pathway inhibitor is an antibody comprising a heavy chain variable region having at least 90% sequence identity to SEQ ID NO:7 and a light chain variable region having at least 90% sequence identity to SEQ ID NO:8.

23. The method of any one of claims 1-22, wherein the Wnt pathway inhibitor is an antibody comprising a heavy chain variable region comprising SEQ ID NO:7 and a light chain variable region comprising SEQ ID NO:8.

24. The method of any one of claims 1-23, wherein the Wnt pathway inhibitor is an antibody comprising a heavy chain comprising SEQ ID NO: 11 and a light chain comprising SEQ ID NO:12.

25. The method of any one of claims 1-23, wherein the Wnt pathway inhibitor is an antibody comprising a heavy chain variable region and a light chain variable region encoded by the plasmid deposited with ATCC as PTA-9541.

26. The method of any one of claims 16-25, wherein the antibody is a monoclonal antibody, a recombinant antibody, a chimeric antibody, a bispecific antibody, a humanized antibody, a human antibody, or an antibody fragment comprising an antigen-binding site.

27. The method of any one of claims 1-16, wherein the Wnt pathway inhibitor is antibody OMP-18R5.

28. The method of any one of claims 1-27, wherein the treatment with a Wnt pathway inhibitor is in combination with at least one additional therapeutic agent.

29. The method of claim 28, wherein the additional therapeutic agent is a chemotherapeutic agent.

30. The method of claim 28, wherein the additional therapeutic agent is a taxane.

31. The method of claim 28, wherein the additional therapeutic agent is paclitaxel or a derivative thereof.

32. The method of claim 28, wherein the additional therapeutic agent is docetaxel.

33. The method of claim 28, wherein the additional therapeutic agent is a platinum complex.

34. The method of claim 28, wherein the additional therapeutic agent is cisplatin or carboplatin.

35. The method of claim 28, wherein the additional therapeutic agents are cisplatin or carboplatin and pemetrexed.

36. The method of any one of claims 1-35, wherein the sample is a tissue sample or a tumor biopsy.

37. The method of any one of claims 1-36, wherein the sample is a formalin-fixed paraffin embedded (FFPE) sample.

38. A kit comprising a reagent for detecting LEF1 in a sample, wherein the reagent comprises primers and probes specific for LEF1.

39. A kit comprising a reagent for detecting LEF1 in a sample, wherein the reagent comprises an antibody that specifically binds LEF1.
